(19) United States
(12) Patent Application Publication
(10) Pub. No.: US 2026/0240996 A1
(43) Pub. Date: Aug. 20, 2026

(54) COMPOSITION OF SELECTED TUMOR INFILTRATING LYMPHOCYTES AND RELATED METHODS OF PRODUCING AND USING THE SAME

(71) Applicant: H. LEE MOFFIT CANCER CENTER AND RESEARCH INSTITUTE, INC., Tampa, FL (US)

(72) Inventors: David STOJDL, Ottawa (CA); Larissa A. PIKOR, Ottawa (CA); James Kenneth NIKOTA, Ottawa (CA); Christophe PEDROS, La Jolla, CA (US); Barbara SENNINO, La Jolla, CA (US)

(21) Appl. No.: 19/164,791

(22) PCT Filed: Mar. 12, 2024
(86) PCT No.: PCT/US2024/019604

§ 371 Date: Sep. 12, 2025

Related U.S. Application Data
(60) Provisional application No. 63/489,999, filed on Mar. 13, 2023.

(51) Int. Cl. A61K 40/11; A61K 40/42; A61P 35/00

(52) U.S. Cl. CPC A61K 40/11; A61K 40/4201; A61K 40/421; A61K 40/4224; A61P 35/00; A61K 2239/28

(57) ABSTRACT

Various embodiments of the invention provide compositions of tumor infiltrating lymphocytes (TILs) enriched in tumor reactive cells. Embodiments also provide methods for manufacturing TILs enriched in tumor reactive cells and uses of the provided enriched tumor reactive TILs for treating cancer in a human or other subject. According to an embodiment, a pharmaceutical T lymphocyte infiltrating (TIL) composition enriched in tumor reactive T cells, comprises an oligoclonal population of tumor infiltrating T cells comprising CD4+ and CD8+ T cells from a tumor, wherein up to 40 clones make up at least 40% of the TCR frequency in the population. Embodiments of the invention are particularly useful for treating tumors that are or have become resistant or refractory to conventional chemotherapy.

### CROSS-REFERENCE TO RELATED APPLICATIONS

This application claims priority from U.S. provisional application 63/489,999, filed Mar. 13, 2023, entitled “COMPOSITION OF SELECTED TUMOR INFILTRATING LYMPHOCYTES AND RELATED METHODS OF PRODUCING AND USING THE SAME”, the contents of which are incorporated by reference in its entirety.

### FIELD

Embodiments of the invention relate to compositions of tumor infiltrating lymphocytes (TILs) enriched in tumor reactive cells. Embodiments of the invention also relate to methods for manufacturing TILs enriched in tumor reactive cells and uses of the provided enriched tumor reactive TILs for treating cancer in a subject.

### BACKGROUND

Clinical studies have demonstrated that T cells isolated from surgically resected tumor possess T-cell receptors (TCRs) that recognize tumor cells and expanding these reactive tumor infiltrating lymphocyte (TIL) populations and re-infusing them into the patient can in some cases result in a dramatic clinical benefit. However, a major obstacle to applications of such cells in cell therapy is the difficulty in obtaining such cells and compositions with desirable features. For example, existing methods for producing TIL therapies for use in cancer is lengthy and compositions may contain a low number of reactive cells that are not suited for commercial applications. Accordingly, there is a need for improved TIL compositions and methods for obtaining and manufacturing cell compositions containing tumor-reactive T cells for therapeutic use. Provided herein are embodiments that meet such needs.

### SUMMARY

In some aspects, provided herein is a pharmaceutical T lymphocyte infiltrating (TIL) composition enriched in tumor reactive T cells, the composition comprising a population of tumor infiltrating T cells comprising CD4+ and CD8+ T cells from a tumor, wherein at least 90% of cells in the composition are CD3+ T cells and less than about 5% of the population are T regulatory cells.

In some aspects, provided herein is a pharmaceutical T lymphocyte infiltrating (TIL) composition enriched in tumor reactive T cells, the pharmaceutical composition comprising an oligoclonal population of tumor infiltrating T cells comprising CD4+ and CD8+ T cells from a tumor, wherein up to 40 clones make up at least 40% of the TCR frequency in the population. In some of any of the provided embodiments, less than about 5% of the population are regulatory T cells. In some of any of the provided embodiments, less than about 3% of the population are T regulatory cells. In some of any of the provided embodiments, less than about 1% of the population are regulatory T cells. In some of any of the provided embodiments, the T regulatory cell phenotype is characterized by surface marker expression of CD4+ CD8− CD25+ Foxp3+ CD127low.

In some of any of the provided embodiments, T cells of the population express PD-1 and/or CD39. In some of any of the provided embodiments, the percentage of cells that express surface marker PD-1 in the composition, or of viable cells thereof, is about at least 30%, at least 40%, at least 50%, at least 60%, at least 70%, at least 80%, at least 90%, at least 100%, of the cells in the composition. In some of any of the provided embodiments, the percentage of cells that express surface marker CD39 in the composition, or of viable cells thereof, is about at least 30%, at least 40%, at least 50%, at least 60%, at least 70%, at least 80%, at least 90%, at least 100%, of the cells in the composition. In some of any of the provided embodiments, the percentage of cells that express surface marker PD-1 and CD39 in the composition, or of viable cells thereof, is about at least 30%, at least 40%, at least 50%, at least 60%, at least 70%, at least 80%, at least 90%, at least 100%, of the cells in the composition. In some of any of the provided embodiments, at least 90% of the cells in the composition are CD3+ T cells. In some of any of the provided embodiments, at least 95% of the cells in the population are CD3+ T cells. In some of any of the provided embodiments, at least 98% of the cells in the population are CD3+ T cells. In some of any of the provided embodiments, at least 80% of the cells in the composition are CD3+ CD56− T cells.

In some of any of the provided embodiments, greater than 60% of the cells of the population are T effector memory cells. In some of any of the provided embodiments, greater than 75% of the cells of the population are T effector memory cells. In some of any of the provided embodiments, greater than 80% of the cells of the population are T effector memory cells. In some of any of the provided embodiments, greater than 85% of the cells of the population are T effector memory cells. In some of any of the provided embodiments, greater than 90% of the cells of the population are T effector memory cells. In some of any of the provided embodiments, the effector memory phenotype is characterized by surface marker expression of one or more of CD45RA⁻, CD45RO+, CD62L⁻, CCR7−, CD28− and CD27−. In some of any of the provided embodiments, the effector memory phenotype is characterized by surface marker expression CD45RA⁻, CD45RO+, CD62L⁻, and CCR7⁻. In some of any of the provided embodiments, the effector memory phenotype is characterized by surface marker expression CD45RA⁻, CD45RO⁺, CD62L, CCR7⁻, CD28⁻ and CD27⁻. In some of any of the provided embodiments, the effector memory phenotype is characterized by surface marker expression CD45RA⁻ CCR7−⁻.

In some of any of the provided embodiments, up to 40 TCR clonotypes make up at least 50% of the TCR frequency in the population. In some embodiments, the top 40 clonotypes make up at least 50% of the TCR frequency in the population. In some of any of the provided embodiments, up to 40 TCR clonotypes make up at least 60% of the TCR frequency in the population. In some embodiments, the top 40 clonotypes make up at least 60% of the TCR frequency in the population. In some of any of the provided embodiments, the TCR clonotypes exhibit reactivity for at least one CD8 antigen and at least one CD4 antigen. In some of any of the provided embodiments, at least 20% of the CD8+ T cells and/or at least 20% of the CD4+ T cells in the composition exhibit neoantigen reactivity.

In some of any of the provided embodiments, the TIL composition is characterized by at least one of the following criteria in an in vitro autologous tumor assay: i) IFN-γ production that is greater than 2000 pg/mL; ii) granzyme B production in the supernatant that is greater than 200 pg/mL; iii) greater than 10% tumor cell killing. In some aspects, provided herein is a pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least about 90% of cells in the composition are CD3+ T cells and wherein, the TIL composition is characterized by at least one of the following criteria in an in vitro autologous tumor assay: i) IFN-γ production that is greater than 2000 pg/mL; ii) granzyme B production that is greater than 500 pg/mL; iii) greater than 15% killing of the autologous tumor cells. In some of any of the provided embodiments, the TIL composition is characterized by criteria (i) and (ii). In some of any of the provided embodiments, the TIL composition is characterized by criteria (i) and (iii). In some of any of the provided embodiments, the TIL composition is characterized by criteria (ii) and (iii). In some of any of the provided embodiments, the TIL composition is characterized by criteria (i), (ii) and (iii). In some of any of the provided embodiments, IFN-γ production is greater than 3000 pg/mL or greater than 4000 pg/mL. In some of any of the provided embodiments, granzyme B production is greater than 400 pg/mL or greater than 500 pg/mL. In some of any of the provided embodiments, killing of the autologous tumor cells is greater than 40%. In some of any of the provided embodiments, the composition is characterized by a greater number of CD4+ T cells than CD8+ T cells. In some of any of the provided embodiments, a ratio of CD4+ T cells to CD8+ T cells in the composition is between 5:1 to 1:5. In some of any of the provided embodiments, a ratio of CD4+ T cells to CD8+ T cells in the composition is between 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1. In some of any of the provided embodiments, a ratio of CD4+ T cells to CD8+ T cells in the composition is at or about 10:1 to 25:1 In some embodiments the ratio of CD4+ to CD8+ T cells is at or about 20:1. In some of any of the provided embodiments, the number of cells in the composition is a therapeutically effective amount of TILs. In some of any of the provided embodiments, the number of cells in the composition, or of viable cells thereof, is at least 2×10⁷ cells. In some of any of the provided embodiments, the number of cells in the composition, or of viable cells thereof, is between at or about 2×10⁷ cells and 20×10⁹ cells, 2×10⁷ cells and 10×10⁹ cells, 2×10⁷ cells and 2×10⁹ cells, 2×10⁷ cells and 2×10⁸ cells, 2×10⁸ cells and 20×10⁹ cells, 2×10⁸ cells and 10×10⁹ cells, 2×10⁸ cells and 2×10⁹ cells, 2×10⁹ cells and 20×10⁹ cells, 2×10⁹ cells and 10×10⁹ cells, or 10×10⁹ cells and 20×10⁹ cells, each inclusive.

In some of any of the provided embodiments, the pharmaceutical composition is for treatment of a patient's tumor. In some of any of the provided embodiments, the tumor is a colorectal cancer (CRC) tumor, a melanoma tumor, a non-small cell lung cancer (NSCLC) tumor, or an ovarian cancer tumor. In some of any of the provided embodiments, the tumor is from a human subject. In some of any of the provided embodiments, the pharmaceutical composition is for autologous adoptive therapy to the human subject. In some of any of the provided embodiments, the pharmaceutical composition comprises a pharmaceutically acceptable excipient. In some of any of the provided embodiments, the pharmaceutical TIL composition comprises a pharmaceutically acceptable excipient and comprises a therapeutically effective amount of tumor reactive T cells, such as, for example, tumor reactive T cells positive for one or more marker as described herein (e.g. PD-1/CD39) that is sufficient to treat, ameliorate or otherwise beneficially alter the symptoms of a condition, disorder or disease or other indication. In some of any of the provided embodiments, the pharmaceutical composition comprises a cryoprotectant. In some of any of the provided embodiments, the composition is a liquid composition. In some of any of the provided embodiments, the TIL composition provided herein is a liquid form where the concentration or amount of tumor reactive T cells in the composition is a therapeutically effective amount sufficient to treat, ameliorate or otherwise beneficially alter the symptoms of a condition, disorder or disease or other indication. In some of any of the provided embodiments, the composition had been frozen and thawed. In some of any of the provided embodiments, the volume of the composition is between 1 mL and 500 mL. In some of any of the provided embodiments, the composition is frozen. In some of any of the provided embodiments, the TIL composition containing a therapeutically effective dose or amount of tumor reactive T cells is frozen.

In some of any of the provided embodiments, the composition is prepared by selecting cells surface positive for PD-1 and CD39 from cells obtained from a tumor of a donor subject and expanding the cells ex vivo. In some of any of the provided embodiments, the composition is prepared by a method comprising; a. providing dissociated tumor cells from a tumor obtained from a donor subject, wherein the dissociated tumor cells are a first population of cells that comprise CD4+ and CD8+ T cells; b. selecting, from the first population of cells, cells that are surface positive for CD45, PD1 and CD39 and a T cell marker to produce a population of selected T lymphocyte infiltrating cells (TILs); and c. expanding the population of selected TILs by culture with one or more T-cell stimulating agent of lymphocytes under conditions to produce a population of expanded T cells. In some aspects, provided herein is a method of producing a T lymphocyte infiltrating (TIL) composition enriched in tumor reactive T cells, the method comprising: a. providing dissociated tumor cells from a tumor obtained from a donor subject, wherein the dissociated tumor cells are a first population of cells that comprise CD4+ and CD8+ T cells; b. selecting, from the first population of cells, cells that are surface positive for CD45, PD1 and CD39 and a T cell marker to produce a population of selected T lymphocyte infiltrating cells (TILs); and c. expanding the population of selected TILs by culture with one or more T-cell stimulating agent of lymphocytes under conditions to produce a population of expanded T cells. In some of any of the above embodiments, the T cell marker is CD4 or CD8.

In some of any of the provided embodiments, the dissociated tumor cells is a single cell suspension processed by homogenization and enzymatic digestion of one or more tumor fragments from the resected tumor. In some of any of the provided embodiments, the one or more T-cell stimulating agent is selected from one or more of allogenic feeder cells, anti-CD3 antibody, and recombinant IL-2. In some of any of the provided embodiments, the enzymatic digestion is by incubation with a collagenase and a hyaluronidase. In some embodiments, the collagenase is at a concentration of about 10 mg/ml. In some embodiments, the hyaluronidase is at a concentration of about 10 mg/ml. In some of any of the provided embodiments, the concentration of the first population of cells is between 5×10⁶ cells/mL and 50×10⁶ cells/mL. In some embodiments, the concentration of the first population of cells is 20×10⁶ cells/mL. In some of any of the provided embodiments, selecting cells is performed using a microfluidics chip based cell sorting comprising at least 4 fluorescence detectors. In some of any of the provided embodiments, selecting the cells comprises sorting the cells positive for the at least 4 fluorescence signal based on a fluorescence minus one (FMO) cocktail. In some of any of the provided embodiments, the sorting is performed at a rate of 5,000 events per second to 10,000 events per second, optionally at about 6,000 events per second.

In some aspects, provided herein is a method of treating a subject having a cancer, the method comprising administering to a subject having a tumor a therapeutic dose of any composition provided herein. In some of any of the provided embodiments, the therapeutically effective dose is between about 1×10⁹ and 10×10⁹ T cells. In some of any of the provided embodiments, the therapeutically effective dose is from more than 1 million to less than 100 million T cells per kilogram of body weight. In some of any of the provided embodiments, the therapeutically effective dose is from more than 1 million to less than 10 million T cells per kilogram of body weight. In some of any of the provided embodiments, the therapeutically effective dose is from at or about 10 million to at or about 50 million T cells per kilogram of body weight. In some of any of the provided embodiments, the cells of the therapeutic composition are autologous to the subject.

### BRIEF DESCRIPTION OF THE DRAWINGS

FIG. 1 is a schematic of a tumor-infiltrating lymphocyte (TIL) manufacturing process using direct selection of TILSs co-expressing PD1+CD39+ surface markers.

FIG. 2A depicts the percentage of TIL co-expressing PD1+ and CD39+ in fresh or frozen single cell suspensions from different cancer indications. Each dot represents a patient sample.

FIG. 2B depicts expansion of PD1+CD39+ selected TILs after a 14-day REP protocol (in the presence of irradiated PBMCs at a 200:1 iPBMC:TIL ratio, 30 ng/ml OKT3 and IL-2 at 3000 IU/ml) where the expansion is calculated as the fold increase between the number of cells after sorting (day 0) and the number of cells harvested at the end of the 14-day REP. Each dot represents data from a patient tumor.

FIG. 3 depicts the proportion of CD3+ T cells (left) and the proportion of CD4+ and CD8+ T cell subsets (right) within the CD3+ T cell population in PD1+CD39+ selected TIL product (end of REP); each dot represents a different patient tumor.

FIG. 4A depicts the percentage of cells expressing CD3+ CD56− (T cells), CD56+ CD3− (NK cells) or CD3+ CD56+ cells. Each dot represents a different selected TIL product from a patient sample.

FIG. 4B depicts the percentage of CD4+ T cells, CD8+ T cells and regulatory T cells (Tregs) within the CD3+ T cell population. Each dot represents a selected TIL product from a patient sample. Samples from the selected TIL product of different indications are represented using different shapes: Lung-circle, Colorectal-square, Kidney-triangle, Melanoma-star, endometrial-diamond.

FIG. 5 depicts the percentage of effector memory T cells (Tem; CD45RA− CCR7−), central memory T cells (Tcm; CD45RA− CCR7+), effector memory T cells expressing CD45RA (Temra; CD45RA+CCR7−), and naïve/stem-cell memory T cells (Tnaive/Scm; CD45RA− CCR7−) within the CD4 (grey bars) and CD8 T cell (white bars) populations. Samples from the selected TIL product of different indications are represented using different shapes: Lung-circle, Colorectal-square, Kidney-triangle, Melanoma-star, endometrial-diamond.

FIG. 6 depicts results of single cell RNA sequencing that was performed on unselected and PD1+CD39+ selected TILs at the end of expansion for TCR clonotypes. Graphs show the diversity and abundance of TCR clonotypes in unselected and PD1+CD39+ selected TILs for each patient. Within each sample set, each different TCRs clonotypes are represented by a segment of the bar. Lines connecting clonotypes between the unselected and PD1+CD39+ samples indicate shared TCR clonotypes between the TIL products. The frequency of the Top 40 most abundant clonotypes within each sample set are displayed.

FIG. 7 depicts IFNγ and granzyme B production by PD1+CD39+ selected TIL product and unselected TIL after overnight stimulation with anti CD3/CD28 antibodies (i.e., polyclonal stimulation (CD3/CD8). (ns=No significant difference).

FIG. 8 depicts IFNγ and granzyme B production by PD1+CD39+ selected TIL product and unselected TIL after overnight stimulation with autologous tumor cells (CD45− DTCs; i.e., autologous tumor material stimulation) (left) and percentage of autologous tumor cell killing induced by PD1+CD39+ selected TILs or unselected TILs (right). Cell killing data was normalized based on the viability of tumor cell alone cultures (0% killing). Dots represent technical replicates; error bars display standard deviation of triplicates. **=p <0.01, Ordinary One-Way ANOVA with Tukey's correction.

FIG. 9 depicts IFNγ and granzyme B production by PD1+CD39+ selected TIL product and unselected TIL after overnight stimulation with anti CD3/CD28 antibodies (i.e., polyclonal stimulation (CD3/CD8). (ns=No significant difference).

FIG. 10 depicts IFNγ and granzyme B production by PD1+CD39+ selected TIL product and unselected TIL after overnight stimulation with autologous tumor cells (CD45− DTCs; i.e., autologous tumor material stimulation) (left) and percentage of autologous tumor cell killing induced by PD1+CD39+ selected TILs or unselected TILs (right). Cell killing data was normalized based on the viability of tumor cell alone cultures (0% killing). Dots represent technical replicates; error bars display standard deviation of triplicates. **=p <0.01, Ordinary One-Way ANOVA with Tukey's correction.

### DETAILED DESCRIPTION

Provided herein are method for manufacturing T cells. Such methods include, but are not limited to the steps of (1) selecting, from a population of cells containing T lymphocytes obtained from a donor subject, cells positive for an exhaustion marker from among PD-1 and/or CD39; and (2) stimulating the population by incubation or culture of selected cells with one or more T-cell stimulating agents of lymphocytes to produce a population of expanded T cells. In some embodiments, the methods for selection and/or stimulation are performed in a closed system. In some embodiments, only a single expansion step is carried out in the method.

In accordance with embodiments herein, methods or processes for manufacturing T cell preparations are provided which may be useful for treating patients with a pathological disease or condition. In contrast to known production methods, the methods and processes described herein can be completed in a significantly shorter time and recover a higher number of T cells, thereby offering a significant advantage to bring cells into the clinic in therapeutic doses. Also provided herein are populations of T cells produced by methods described herein and pharmaceutical compositions thereof.

The provided methods relate to producing a T cell therapy reactive to tumor-associated antigens. Cancer cells accumulate lots of different DNA mutations as part of the tumorigenic process. These mutations can cause amino acid changes in protein coding regions. For a mutation to be recognized by the immune system the protein needs to be processed intracellularly and presented on the surface with the Major Histocompatibility Complex (MHC). Peptide neoantigens (also referred to herein as neoepitopes or peptide neoepitopes) are the mutant peptides presented by the MHC complex that can be recognized by a T-cell via TCR binding. In order for the immune system to recognize the mutation, it must be expressed on the surface of the cancer cell via the MHC complex and the T cell must have a TCR that recognizes the mutated peptide. These neoantigens may be presented by MHC class I and MHC class II, and are recognized by CD8+ and CD4+ T cells respectively.

In some embodiments, the method described may be used to manufacture T cells which express cell surface receptors. The cell surface receptor may be a T cell receptor (TCR) or novel group of TCRs. In particular embodiments of the provided methods, the population of T cells is or includes reactive T cells that express cell surface receptors, such as a T cell receptor (TCR), able to recognize peptide antigens on the surface of a target cells. Specifically, for an antigen to be recognized by the immune system the protein needs to be processed intracellularly to peptide fragments that are then presented on the surface with the Major Histocompatibility Complex (MHC). A TCR has two protein chains, which are designed to bind with specific peptides presented by a major histocompatibility complex (MHC) protein on the surface of certain cells. Since TCRs recognize peptides in the context of MHC molecules expressed on the surface of a target cell, TCRs have the potential to recognize antigens not only presented directly on the surface of target cells, e.g. cancer cells, but also presented by antigen-presenting cells, such as are present in tumor, inflammatory and infected microenvironments, and in secondary lymphoid organs. Reactive T cells expressing such cell surface receptors may be used to target and kill any target cell, including, but not limited to, infected cells, damaged cells, or dysfunctional cells. Thus, according to the embodiments described herein, the manufactured T cells expressing the cell surface receptor may be used to target and kill any target cell, including, but not limited to, infected cells, damaged cells, or dysfunctional cells. Examples of such target cells may include cancer cells, virally infected cells, bacterially infected cells, dysfunctionally activated inflammatory cells (e.g., inflammatory endothelial cells), and cells involved in dysfunctional immune reactions (e.g., cells involved in autoimmune diseases).

In some embodiments, a “T cell receptor” or “TCR” is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a T cells (or T lymphocytes) where it is generally responsible for recognizing antigens bound to major histocompatibility complex (MHC) molecules.

In some aspects, the reactive T cells are tumor-reactive T cells that recognize a cancer neoantigen. The majority of neoantigens arise from passenger mutations, meaning they do not infer any growth advantage to the cancer cell. A smaller number of mutations actively promote tumor growth, these are known as driver mutations. Passenger mutations are likely to give rise to neoantigens that are unique to each patient and may be present in a subset of all cancer cells. Driver mutations give rise to neoantigens that are likely to be present in all the tumor cells of an individual and potentially shared. In some embodiments of the provided method, the population of T cells contain tumor-reactive T cells that can recognize neoantigens containing passenger and/or driver mutations.

In particular aspects, the provided methods can be used for the ex vivo production of a T cell therapy, including for the ex vivo expansion of autologous tumor-reactive T cells. In some aspects, neoantigens are ideal targets for immunotherapies because they represent disease-specific targets. For example, such antigens generally are not present in the body before the cancer developed and are truly cancer specific, not expressed on normal cells and are not subjected to off target immune toxicity. Thus, the unique repertoire of neoantigens specific to the patient can elicit a strong immune response specific to the cancer cells, avoiding normal cells. This is an advantage over other cell therapy targets that may not be disease-specific targets, since even low levels of target antigen on normal cells can lead to severe fatal autoimmune toxicity in the context of engineered therapies that target common antigens. For example an anti MAGE-A3-TCR program in melanoma patients was halted due to study related deaths attributed to cross reactivity with a similar target MAGE-A12, which is expressed at a low level in the brain. A significant challenge in cancer immunotherapy has been the identification of cancer targets.

Recent clinical studies have demonstrated that T cells isolated from surgically resected tumors possess TCRs that recognize neoantigens, and expanding these neoantigen reactive TIL populations and re-infusing them into the patient can in some cases result in a dramatic clinical benefit. This personalized therapy has generated remarkable clinical responses in certain patients with common epithelial tumors.

Existing methods for obtaining and generating tumor-reactive T cells are not entirely satisfactory. For example, direct isolation of tumor-reactive T cells from a subject without expansion is not feasible because therapeutically effective amounts of such cells cannot be obtained. As an alternative, attempts have been made to identify TCRs specific to a desired neoantigen for recombinant engineering of the TCR into T cells for use in adoptive cell therapy methods. Such approaches, however, produce only a single TCR against a specific neoantigen and thereby lack diversity to recognize a broader repertoire of multiple tumor-specific mutations. Other methods involve bulk expansion of T cells from a tumor source, which has the risk of expanding T cells that are not reactive to a tumor antigen and/or that may include a number of bystander cells that could exhibit inhibitory activity. For example, tumor regulatory T cells (Tregs) are a subpopulation of CD4⁺ T cells, which specialize in suppressing immune responses and could limit reactivity of a T cell product. These further approaches that have sought to expand tumor-reactive T cells ex vivo are not selective such that non-reactive T cells in the culture may preferentially expand over reactive T cells resulting in a final product that lacks satisfactory reactivity and/or in which the number of tumor-reactive T cells remains insufficient. Methods to produce tumor-reactive T cells for therapy are needed.

The provided embodiments relate to improved methods for identifying and expanding T cells ex vivo, including tumor-reactive T cells, for use in T cell therapy. In some embodiments, the provided methods improve or increase the growth and survival of T cells, such as tumor-reactive T cells, outside of the body. In particular embodiments, the methods enrich for expansion of reactive T cells compared to non-reactive T cells and promote their survival and growth in culture ex vivo. In some embodiments, the resulting methods can be carried out in a closed system. The methods in some embodiments are carried out in an automated or partially automated fashion.

The provided methods contemplate that selection of cells during one or more steps of an ex vivo process for manufacturing tumor reactive T cells based on expression of CD45, CD4 or CD8, and one or more exhaustion marker PD-1, and/or CD39 will result in an improved TIL therapy enriched in tumor reactive T cells with high potential for therapeutic efficacy for treating certain cancers. The provided methods result in a product containing tumor reactive T cells that can target many mutations and/or that contains an oligoclonal population of TCRs that are reactive to different tumor antigens. Thus, such tumor reactive T cells offer advantages compared to existing methods in which cells are transduced to express a single neoepitope reactive TCR.

PD-1 and CD39 are each checkpoint molecules that also can represent markers of exhausted T cells. They also are markers that are activation markers or upregulation markers in that their expression is increased upon tumor reactivity, which is a natural mechanism of immune suppression of the host immune response. For instance, the immune system is designed to shut itself off to avoid an overactive immune response in order to avoid inflammatory and autoimmune responses. In this way, an immune response is initially developed against cancer but this can be thwarted by the upregulation of certain checkpoint molecules, like PD-1 and CD39, that can inhibit the immune response. As these are markers that are upregulated on cells in which an immune response is being developed, it is contemplated by the provided methods that such markers serve as powerful markers for specifically enriching for tumor reactive T. By specifically selecting for tumor reactive cells based on these activation markers, the provided methods avoid bulk expansion of T cells from a tumor source that would include a number of bystander cells that are not tumor reactive or that could exhibit inhibitory activity, such as Tregs.

In some embodiments, expression of one or more PD-1 and/or CD39 are used to enrich TIL immediately after tumor dissociation, either at the endpoint of tumor fragment culture or immediately after mechanical/enzymatic creation of a single cell suspension from tumor fragments. In some embodiments, PD-1 and/or CD39 expressing cells, among cells that also express CD45 and CD4 or CD8, are isolated from either tumor fragment cultures or single cell suspensions generated through enzymatic digestion. In aspects of the provided methods, after selection, selected cells can be expanded in the presence of one or more T cell stimulating agent. In some embodiments, the T cell stimulating agent can include any one or more recombinant cytokines IL-2, IL-7, IL-15, IL-21, IL-25, IL-23, IL-27 or IL-25, such as generally at least IL-2 or IL-15. In some embodiments, the T cell stimulating agent can include any one or more recombinant cytokines IL-2, IL-7, IL-15, or IL-21. In some embodiments, the T cell stimulating agent can include recombinant cytokine IL-2, In some embodiments, the T cell stimulating agent can further include an anti-CD3 antibody (e.g. OKT3). In some embodiments, the T cell stimulating agents include an anti-CD3 antibody (OKT3) and/or a recombinant cytokine such as IL-2, IL-7, IL-15, IL-21, IL-25, IL-23.

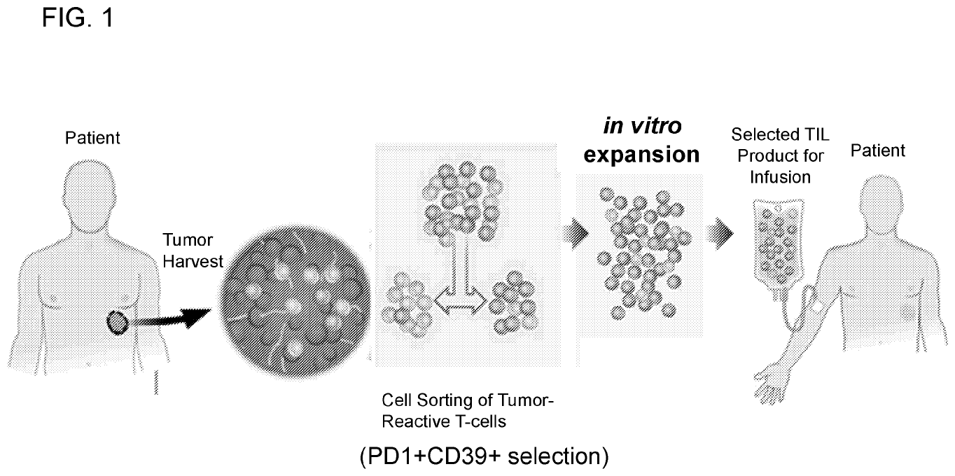

FIG. 1 depicts a schematic of an exemplary process for manufacturing a T cell therapeutic composition in accord with the provided methods. In the exemplary process a tumor sample is obtained from a patient. In some cases, TILs are enriched from the sample by selection for cells positive for one or more marker associated with tumor reactive cells (hereinafter “selection marker”), such as an exhaustion marker such as PD-1/CD39. In some cases, a population of T cells from the patient, e.g. containing tumor infiltrating lymphocytes (TIL) or enriched to TILs, is stimulated under conditions to expand the cells, tumor-reactive T cells or T cells positive for one or more marker associated with tumor reactive cells (hereinafter “selection marker”), such as an exhaustion marker such as PD-1/CD39 can be selected and cultured under conditions for expansion in accord with the provided methods, such as incubation with a T cell stimulatory agent(s) (e.g. recombinant IL-2, anti-CD3). The culturing can be carried out in the presence of one or more recombinant cytokines (e.g. IL-2) to support proliferation and expansion of cells. The process can be carried out in the presence of serum-free media containing nutrients.

One or more or all of the steps can be carried out in a closed system, such as without exposure of cells to the environment. Upon reaching a therapeutic dose or a threshold number of cells, the cells can be harvested and formulated, in some cases concentrated or cryopreserved, and used for administration to a subject such as by infusion.

The provided methods offer advantages compared to existing methods for producing and expanding TILs because the provided methods involve steps to enrich for tumor reactive cells, such as by selecting for T cells that are likely or suspecting of being enriched in tumor-reactive T cells. In some cases, the methods can enrich for tumor reactive T cells by selection of CD45+ cells that are PD-1+CD39+, and also minimize bystander cells such as regulatory T cells. By virtue of this process, the initial small population of tumor reactive T cells expanded from the biological sample (e.g. tumor) are enriched for cells that are or likely to be tumor reactive cells before a subsequent second expansion step, thereby promoting preservation and expansion of cells of interest and limiting expansion of bystander T cells that are not reactive to a tumor antigen and/or that may include cells that exhibit inhibitory activity. This is in contrast to existing methods that involve passive expansion of bulk T cells in which all T cells from a tumor are subjected to a first initial expansion, e.g. with high IL-2 concentrations, followed by a second rapid expansion of T cells present after the initial expansion. In such other methods, while total viable cells (TVC) can be greatly expanded by these alternative processes, there is no step of actively ensuring that tumor reactive T cells are predominantly propagated. In aspects of the provided methods, all steps of the method are carried out in a closed system.

The provided methods include one or more features that provide for or relate to an improved, more efficient and/or more robust process for producing a tumor-reactive T cell therapeutic composition ex vivo. In particular, the disclosure relates to methods that provide advantages over available methods for producing a TIL therapeutic cell composition. Such advantages include, for example, reduced cost, streamlining, improved enrichment of tumor-reactive T cells in the therapeutic composition, and increased efficacy of the therapeutic composition, including among different subjects and tumor conditions.

In aspects of the provided methods, expansion can be carried out with relatively lower concentrations of recombinant IL-2 during one or both expansion steps with success. Many existing methods use high concentrations of IL-2 of 6000 IU/mL for T cell expansion of TIL. However, high IL-2 concentrations can increase the cost of the process and may be limiting. In some cases, high IL-2 concentrations may lead to negative impacts on T cell differentiation by driving effector T cell differentiation over early memory T cells that may be more desirable in a therapeutic T cell composition. The provided methods can be carried out with concentrations that are lower than 6000 IU/mL, such as concentrations less than at or about 3000 IU/mL.

In embodiments of the provided methods, the population of T cells is obtained from a biological sample known to contain T cells. In some embodiments, the population of T cells is enriched from a biological sample from a subject, in particular a human subject. The biological sample can be any sample containing a bulk population of T cells. In some embodiments, the biological sample is or includes peripheral blood mononuclear cells. In some embodiments, the biological sample is a peripheral blood or serum sample. In some embodiments, the biological sample is a lymph node sample. In some embodiments, the biological sample is a tumor sample. In some aspects, the bulk T cells can include tumor-infiltrating T cells (TILs). In some embodiments, the subject is a human subject. In some embodiments the subject is a subject having a cancer, viral infection, bacterial infection, or is a subject with an inflammatory condition. In particular embodiments, the subject has a cancer.

In aspects of the provided methods, the starting source of cells (input sample) in the method can be tumor fragments (e.g. 1-8 mm diameter fragments) or can be a single cell suspension preparation from enzymatic digestion of tumor fragments. While certain sources may be superior for some tumor types, both fragments and single cell suspensions can support T cell expansion and enrichment of tumor-reactive T cells. In some cases, the tumor cell source can be chosen depending on the tumor type or cancer, such as to optimize or increase expansion and enrichment of tumor-reactive T cells from the tumor. In one example, the cancer is a melanoma and the starting population of lymphocytes are tumor fragments, such as from a resected tumor. In another example, the cancer is a colorectal cancer and the starting population of lymphocytes is a single cell suspension obtained by enzymatic digestion, e.g. collagenase and/or hyaluronidase, of tumor fragments.

In some embodiments, the methods produce or expand T cells for use in adoptive cell therapy for treating a disease or condition in which cells or tissue associated with the disease or condition is known or suspected of expressing an antigen target recognized by the T cells. In some embodiments, the T cell therapy is autologous to the subject. In some embodiments, the T cell therapy is allogeneic to the subject.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Compositions and Pharmaceutical Formulations

Provided herein are TIL compositions that are enriched for tumor reactive T cells. In some embodiments, the TIL compositions contain primary T cells from a tumor from a subject that have been selected based on surface expression of PD-1 and CD39 and expanded ex vivo. In some embodiments, provided TIL compositions containing selected and expanded T cells can be produced by the provided ex vivo methods for producing TIL compositions.

In some embodiments, the provided TIL composition is a multiclonal population that exhibits TCR diversity and enrichment of T cell receptors (TCRs) reactive to tumor antigens. In some embodiments, the provided TIL composition is an oligoclonal population that exhibits TCR diversity and enrichment of different TCR clonotypes. In some embodiments, the TIL composition contains up to 40 different TCR clonotypes (e.g. Top40 clones) that make up at least 40% of the TCR frequency in the population. In some embodiments, the TIL composition contains up to 40 different TCR clonotypes that make up at least 50% of the TCR frequency in the population. In some embodiments, the TIL composition contains up to 40 different TCR clonotypes that make up at least 60% of the TCR frequency in the population. In some embodiments, the TIL composition contains up to 40 different TCR clonotypes that make up at least 70% of the TCR frequency in the population. In some of any of the above embodiments, the number of TCR clonotypes making up the percentage is 10 to 40 different TCR clonotypes. In some of any of the above embodiments, the number of TCR clonotypes making up the percentage is 20 to 40 different TCR clonotypes. In some of any of the above embodiments, the number of TCR clonotypes making up the percentage is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 different TCR clonotypes.

In some embodiments, the TIL composition contains 20 to 40 different TCR clonotypes that make up at least 40% of the TCR frequency in the population. In some embodiments, the TIL composition contains 20 to 40 different TCR clonotypes that make up at least 50% of the TCR frequency in the population. In some embodiments, the TIL composition contains 20 to 40 different TCR clonotypes that make up at least 60% of the TCR frequency in the population. In some embodiments, the TIL composition contains 20 to 40 different TCR clonotypes that make up at least 70% of the TCR frequency in the population.

In some embodiments, the top40 TCR clonotypes make up at least 75% of the TCR frequency in the population. In some embodiments, the top 40 TCR clonotypes make up at least 80% of the TCR frequency in the population. In some embodiments, the top40 TCR clonotypes make up at least 85% of the TCR frequency in the population. In some embodiments, the top40 TCR clonotypes make up at least 90% of the TCR frequency in the population.

In some embodiments, the neoantigen reactivity of the TCR clonotypes is reactivity to at least one CD4 antigen and at least one CD8 antigen. In some embodiments, the neoantigen reactivity of the TCR clonotypes is for at least 2 peptide antigens in which as least one peptide antigen is a CD4 antigen and at least one peptide antigen is a CD8 antigen.

Various methods for assessing the TCR repertoire for clonotype identification and TCR repertoire analysis are known (see e.g. Rosati et al. (2017) BMC Biotechnology, 17:61; Friedensohn et al. (2016) Trends in Biotechnology, 35:203-214). In some aspects, the methods involve high-throughput or next-generation sequencing methods. In some embodiments, the frequency and variety of different clones present in the population or composition can be determined. In some embodiments, the compositions can be assessed the clonality, clonal diversity or clonal heterogeneity of the cells in the population of the composition of cells, for example, based on the determined frequency and/or variety of clonotypes present in the population or composition. In some embodiments, single-cell sequencing methods are carried out to identify a clonotype on a particular cell. In certain aspects, paired αβ TCR sequencing methods are used (see e.g. WO2017053902A1). In some embodiments, sequencing methods are carried out on DNA, such as genomic DNA or complementary DNA. In some embodiments, sequencing methods are carried out on RNA. In some embodiments, high-throughput or next-generation sequencing of TCR sequences or by sequencing the whole genome or transcriptome (e.g., RNAseq). In some aspects, the methods used are RNAseq-based methods.

In some embodiments, T cell clonotype assessment and clonality and diversity in various T cell populations or compositions or samples containing T cells, are determined using high-throughput sequencing of all or a portion of the TCR genes or based on sequences obtained from high-throughput whole genome or transcriptome analysis, on the population or composition of cells, and/or in a single cell. In some embodiments, the provided methods can include various features of the methods as described in WO2016/044227, WO2016/176322, WO2012/048340, WO2012/048341, WO2014/144495, WO2017/053902, WO2017/053903 or WO2017/053905, each incorporated by reference in their entirety.

The clonotypes of a cell or the clonotypes present in a population or composition of cells, in some examples, may be determined by TCR sequencing. In some embodiments, sequencing methods that can be employed include high-throughput or next-generation sequencing as is known in the art. In some aspects, next-generation sequencing methods can be employed, using genomic DNA or cDNA from T cells, to assess the TCR repertoire, including sequences encoding the complementarity-determining region 3 (CDR3). In some embodiments, whole transcriptome sequencing by RNAseq can be employed. In some aspects, the TCR repertoire information, e.g., TCR sequences and relative frequency, can be constructed or extracted from whole transcriptome sequencing (e.g., by RNAseq). For example, in some aspects, computational methods such as MIXCR (Such as those described in Bolotin et al. Nature Methods 12 (2015) 380-381, Bolotin et al., Nature Biotechnology 35 (2017) 908-911) or IMREP (Mangul et al., bioRxiv (2017) 089235) can be utilized to determine the repertoire TCR sequences or a portion thereof (e.g., CDR3) from whole transcriptome RNAseq results. In some embodiments, single-cell sequencing methods can be used. In some embodiments, clonotypes can be assessed or determined by spectratype analysis (a measure of the TCR Vβ, Vα, Vγ, or Vδ chain hypervariable region repertoire). Clonotypes can also be determined by generation and characterization of antigen-specific clones to an antigen of interest.

In some embodiments, T cell clonotype assessment are determined using high-throughput sequencing of all or a portion of the TCR genes or based on sequences obtained from high-throughput whole genome or transcriptome analysis, on the population or composition of cells, and/or in a single cell. In some embodiments, bulk sequencing of targeted sequences (e.g., TCR chains or portion thereof) or bulk whole genome or transcriptome sequencing (e.g., by RNAseq) can be used to determine the clonotypes present in the cells in the population or composition. In some aspects, T cell clonotype assessment can involve sequencing of a portion of the variable region of one or more native TCR chains, such as the complementarity-determining region 3 (CDR3). In some aspects, single cell sequencing can be employed. In some embodiments, the provided methods can include various features of the methods as described in WO2016/044227, WO2016/176322, WO2012/048340, WO2012/048341, WO2014/144495, WO2017/053902, WO2017/053903 or WO2017/053905, each incorporated by reference in their entirety. In some embodiments, for target TCR molecules, the genes encoding chains of a TCR can be obtained from genomic DNA or mRNA of immune cells or T cells.

In some embodiments, the composition exhibits clonal diversity, i.e. is multiclonal, such as is oligoclonal. In some cases, the clonal diversity is determined based on the relative frequency of the one or more clonotypes and/or one or more TCR sequences. In some embodiments, the determining the clonal diversity is represented as clonality, Shannon-adjusted clonality or top 25 clonality of each of the plurality of samples. In some embodiments, the determining the clonal diversity is represented as Shannon-adjusted clonality in a composition.

In some embodiments, the cells of the provided TIL compositions exhibit one or more phenotypic or functional markers. In some cases, such cells include cells positive or negative for one or more phenotypic marker or functional feature or attribute.

As used herein, a statement that a cell or population of cells is “positive” for a particular marker, function or attribute refers to the detectable presence on or in the cell of a particular marker, such as a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker, function or attribute refers to the absence of substantial detectable presence on or in the cell of a particular marker, such as a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Exemplary markers, functions and attributes of provided TIL compositions are described below. In some embodiments, the TIL composition is characterized by any one or more of such features, such as 2, 3, 4, 5 or more of such features. For instance, a provided TIL composition may be characterized by presence or absence of one or more T cell markers, effector memory phenotype markers, exhaustion markers, the ability to produce or secrete cytokines and/or the ability to produce or secrete a cytotoxic factor, such as described below. Any 2, 3, 4, 5 or more of any of such features may be present in a TIL composition as described.

In some embodiments, a provided TIL composition comprises CD3+ T cells as a percentage of total cells in the population that is greater than or greater than about 85%, such as greater than or greater than about 90%, such as greater than or greater than about 95%, greater than or greater than about 97% or greater than or greater than about 98%. In some embodiments, a provided TIL composition comprises CD3+ T cells as a percentage of total cells in the population that is greater than or greater than about 90%. In some embodiments, a provided TIL composition comprises CD3+ T cells as a percentage of total cells in the population that is greater than or greater than about 95%. In some embodiments, a provided TIL composition comprises CD3+ T cells as a percentage of total cells in the population that is greater than or greater than about 98%. In some embodiments, the composition contains CD4+ T cells and CD8+ T cells as a percentage of total cells in the population that is greater than or greater than about 85%, greater than or greater than about 90%, greater than or greater than about 95%, greater than or greater than about 97% or greater than or greater than about 98%. In some embodiments, a provided TIL composition comprises CD3+ T cells as a percentage of total cells in the population that is greater than the amount of CD3+ T cells as a percentage of total cells isolated from a patient.

In some embodiments, the composition contains a ratio of CD4+ T cells to CD8+ T cells that is between at or about 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1. In some embodiments, the composition contains a ratio of CD4+ T cells to CD8+ T cells that is at or about 10:1 to 25:1. In some embodiments, the composition contains a ration of CD4+ T cells to CD8+ T cells that is about 20:1.

In some embodiments, among CD3+ T cells in the TIL composition, or CD4+ and/or CD8+ T cell subsets thereof, less than 5% express a marker of a T regulatory cell phenotype, such as less than 4% express a marker of a T regulatory cell phenotype, less than 3% express a marker of a T regulatory cell phenotype, less than 2% express a marker of a T regulatory cell phenotype, less than 1% express a marker of a T regulatory cell phenotype. In some embodiments, among CD3+ T cells in the TIL composition, or CD4+ and/or CD8+ T cell subsets thereof, less than 4% express a marker of a T regulatory cell phenotype. In some embodiments, among CD3+ T cells in the TIL composition, or CD4+ and/or CD8+ T cell subsets thereof, less than 3% express a marker of a T regulatory cell phenotype. In some embodiments, among CD3+ T cells in the TIL composition, or CD4+ and/or CD8+ T cell subsets thereof, less than 2% express a marker of a T regulatory cell phenotype. In some embodiments, among CD3+ T cells in the TIL composition, or CD4+ and/or CD8+ T cell subsets thereof, less than 4% express a marker of a T regulatory cell phenotype less than 1% express a marker of a T regulatory cell phenotype, less than 3% express a marker of a T regulatory cell phenotype. In some embodiments, the T cell regulatory phenotype is characterized by surface marker expression of one or more of CD25+, Foxp3+ and CD1271ow. In some embodiments, the T cell regulatory phenotype is characterized by surface marker expression CD4+ and Foxp3+. In some embodiments, the T cell regulatory phenotype is characterized by surface marker expression CD8-Foxp3+. In some embodiments, the T cell regulatory phenotype is characterized as CD4+ CD8− CD25+Foxp3+ CD1271ow.

In some embodiments, the provided TIL composition includes an increased or greater percentage of cells of a particular phenotype or function compared to an unselected TIL composition. In particular embodiments, reference to the unselected TIL composition refers to a population of TILs expanded from an unselected sample under conditions that are identical or substantially identical to methods for expansion of a selected TIL composition, such as using methods for cell stimulation and expansion described in Section II.C. In particular embodiments, reference to the unselected sample refers to a first population of cells prior to the selection of PD-1 and/or CD39 positive cells (e.g. PD-1 and CD39 positive cells). In some embodiments, the unselected sample is the first population of cells generated as described in section II.A. In some embodiments, the unselected sample is a single cell suspension of dissociated tumor cells. For instance, in some embodiments, an unselected TIL composition refers to a TIL composition that is processed the same or substantially the same to the provided TIL composition generated as described in Sections II.A-C expect that the cells are not selected for PD-1 and CD39 cells from the dissociated tumor cells prior to their ex vivo expansion. Thus, in some cases the unselected TIL composition represents a bulk expanded population of T cells from the tumor cell that are subject to ex vivo expansion without enrichment for tumor-reactive T cells.

In some embodiments, the provided TIL composition includes an increased or greater percentage of CD3+ T cells positive for PD-1 and/or CD39 compared to the percentage of such CD3+ T cells positive for PD-1 and/or CD39 in an unselected TIL composition. In some embodiments, the percentage is increased at least or at least about 2-fold, 3-fold, 4-fold, 5-fold, 10-fold, 20-fold or more.

In some embodiments, the provided composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, CD3+ T cells positive for PD-1 and/or CD39. In some embodiments, the composition comprises more than 30% CD3+ T cells positive for PD-1 and/or CD39. In some embodiments, the composition comprises more than 40% CD3+ T cells positive for PD-1 and/or CD39. In some embodiments, the composition comprises more than 50% CD3+ T cells positive for PD-1 and/or CD39. In some embodiments, the composition comprises more than 60% CD3+ T cells positive for PD-1 and/or CD39. In some embodiments, the composition comprises more than 70% CD3+ T cells positive for PD-1 and/or CD39. In some embodiments, the composition comprises more than 80% CD3+ T cells positive for PD-1 and/or CD39. In some embodiments, the composition comprises more than 90% CD3+ T cells positive for PD-1 and/or CD39. I

In some embodiments, the provided composition comprises more than 30% CD3+ T cells positive for PD-1. In some embodiments, the composition comprises more than 40% CD3+ T cells positive for PD-1. In some embodiments, the composition comprises more than 50% CD3+ T cells positive for PD-1. In some embodiments, the composition comprises more than 60% CD3+ T cells positive for PD-1. In some embodiments, the composition comprises more than 70% CD3+ T cells positive for PD-1. In some embodiments, the composition comprises more than 80% CD3+ T cells positive for PD-1. In some embodiments, the composition comprises more than 90% CD3+ T cells positive for PD-1.

In some embodiments, the provided composition comprises more than 30% CD3+ T cells positive for CD39. In some embodiments, the composition comprises more than 40% CD3+ T cells positive for CD39. In some embodiments, the composition comprises more than 50% CD3+ T cells positive for CD39. In some embodiments, the composition comprises more than 60% CD3+ T cells positive for CD39. In some embodiments, the composition comprises more than 70% CD3+ T cells positive for CD39. In some embodiments, the composition comprises more than 80% CD3+ T cells positive for CD39. In some embodiments, the composition comprises more than 90% CD3+ T cells positive for CD39.

In some embodiments, the provided composition comprises more than 30% CD3+ T cells positive for PD-1 and CD39. In some embodiments, the composition comprises more than 40% CD3+ T cells positive for PD-1 and CD39. In some embodiments, the composition comprises more than 50% CD3+ T cells positive for PD-1 and CD39. In some embodiments, the composition comprises more than 60% CD3+ T cells positive PD-1 and CD39. In some embodiments, the composition comprises more than 70% CD3+ T cells positive for PD-1 and CD39. In some embodiments, the composition comprises more than 80% CD3+ T cells positive for PD-1 and CD39. In some embodiments, the composition comprises more than 90% CD3+ T cells positive for PD-1 and CD39.

In some embodiments, among CD3+ T cells in the TIL composition, or CD4+ and/or CD8+ T cell subsets thereof, greater than 50% express a marker of an effector memory phenotype. In some embodiments, among CD3+ T cells in the TIL composition, or CD4+ and/or CD8+ T cell subsets thereof, greater than about 60% express an effector memory phenotype marker, greater than about 70% express an effector memory phenotype marker, greater than about 80% express an effector memory phenotype marker, or greater than 90% express an effector memory phenotype marker. In some embodiments, among CD3+ T cells in the TIL composition, or CD4+ and/or CD8+ T cell subsets thereof, greater than 60% express a marker of an effector memory phenotype. In some embodiments, the effector memory phenotype is characterized by surface marker expression of one or more of CD45RA−, CD45RO+, CD62L⁻, CCR7−, CD28− and CD27−. In some embodiments, the effector memory phenotype is characterized by surface marker expression CD45RA− and CCR7−. In some embodiments, the effector memory phenotype is characterized by surface marker expression CD45RA− CD45RO+, CD62L⁻, and CCR7⁻. In some embodiments, the effector memory phenotype is characterized by surface marker expression CD45RA⁻, CD45RO+, CD62L⁻, CCR7⁻, CD28⁻ and CD27⁻.

In some embodiments, among CD3+ T cells in the TIL composition, or CD4+ and/or CD8+ T cell subsets thereof, greater than about 50% are CD45RA− and CCR7, greater than about 60% are CD45RA− and CCR7−, greater than about 70% are CD45RA− and CCR7−, greater than about 80% are CD45RA− and CCR7−, or greater than 90% are CD45RA− and CCR7−.

In some embodiments, among CD3+ T cells in the TIL composition, or CD4+ and/or CD8+ T cell subsets thereof, greater than about 10% express a central memory T cell marker, greater than about 15% express a central memory T cell marker, greater than about 20% express a central memory T cell marker, or greater than 25% express a central memory T cell marker. In some embodiments, the central memory T cell marker is CD45RA-CCR7+. In some embodiments, among CD3+ T cells in the TIL composition, or CD4+ and/or CD8+ T cell subsets thereof, greater than about 10% are CD45RA-CCR7+, greater than about 15% are CD45RA-CCR7+, greater than about 20% are CD45RA-CCR7+, or greater than 25% are CD45RA-CCR7+.

In some embodiments, among CD3+ T cells in the TIL composition, or CD4+ and/or CD8+ T cell subsets thereof, greater than about 10% express a central memory T cell marker (e.g. CD45RA-CCR7+) and greater than about 60% express an effector memory phenotype marker (e.g. CD45RA-CCR7−). In some embodiments, among CD3+ T cells in the TIL composition, or CD4+ and/or CD8+ T cell subsets thereof, greater than about 15% express a central memory T cell marker (e.g. CD45RA-CCR7+) and greater than about 60% express an effector memory phenotype marker (e.g. CD45RA-CCR7−). In some embodiments, among CD3+ T cells in the TIL composition, or CD4+ and/or CD8+ T cell subsets thereof, greater than about 20% express a central memory T cell marker (e.g. CD45RA-CCR7+) and greater than about 60% express an effector memory phenotype marker (e.g. CD45RA-CCR7−). In some embodiments, among CD3+ T cells in the TIL composition, or CD4+ and/or CD8+ T cell subsets thereof, greater than about 25% express a central memory T cell marker (e.g. CD45RA-CCR7+) and greater than about 60% express an effector memory phenotype marker (e.g. CD45RA-CCR7−).

In some embodiments, among CD3+ T cells in the TIL composition, or CD4+ and/or CD8+ T cell subsets thereof, greater than about 10% express a central memory T cell marker (e.g. CD45RA-CCR7+) and greater than about 70% express an effector memory phenotype marker (e.g. CD45RA-CCR7−). In some embodiments, among CD3+ T cells in the TIL composition, or CD4+ and/or CD8+ T cell subsets thereof, greater than about 15% express a central memory T cell marker (e.g. CD45RA-CCR7+) and greater than about 70% express an effector memory phenotype marker (e.g. CD45RA-CCR7−). In some embodiments, among CD3+ T cells in the TIL composition, or CD4+ and/or CD8+ T cell subsets thereof, greater than about 20% express a central memory T cell marker (e.g. CD45RA-CCR7+) and greater than about 70% express an effector memory phenotype marker (e.g. CD45RA-CCR7−). In some embodiments, among CD3+ T cells in the TIL composition, or CD4+ and/or CD8+ T cell subsets thereof, greater than about 25% express a central memory T cell marker (e.g. CD45RA-CCR7+) and greater than about 70% express an effector memory phenotype marker (e.g. CD45RA-CCR7−).

In some embodiments, among CD3+ T cells in the TIL composition, or CD4+ and/or CD8+ T cell subsets thereof, greater than about 10% express a central memory T cell marker (e.g. CD45RA-CCR7+) and greater than about 80% express an effector memory phenotype marker (e.g. CD45RA-CCR7−). In some embodiments, among CD3+ T cells in the TIL composition, or CD4+ and/or CD8+ T cell subsets thereof, greater than about 15% express a central memory T cell marker (e.g. CD45RA-CCR7+) and greater than about 80% express an effector memory phenotype marker (e.g. CD45RA-CCR7−). In some embodiments, among CD3+ T cells in the TIL composition, or CD4+ and/or CD8+ T cell subsets thereof, about 20% express a central memory T cell marker (e.g. CD45RA-CCR7+) and about 80% express an effector memory phenotype marker (e.g. CD45RA-CCR7−).

In some embodiments, among CD3+ T cells in the TIL composition, or CD4+ and/or CD8+ T cell subsets thereof, about 10% express a central memory T cell marker (e.g. CD45RA-CCR7+) and about 90% express an effector memory phenotype marker (e.g. CD45RA-CCR7−).

In some embodiments, a provided TIL composition includes about 10-60% tumor-reactive T cells. In some embodiments, a provided TIL composition includes greater than about 15% tumor reactive T cells, greater than about 20% tumor-reactive T cells, greater than about 25% tumor-reactive T cells, greater than about 30% tumor-reactive T cells, greater than about 40% tumor-reactive T cells or greater than about 50% tumor-reactive T cells, or any value between any of the foregoing. In some embodiments, a provided TIL composition that includes tumor reactive T cells in a therapeutically effective amount includes greater than about 15% tumor reactive T cells, greater than about 20% tumor-reactive T cells, greater than about 25% tumor-reactive T cells, greater than about 30% tumor-reactive T cells, greater than about 40% tumor-reactive T cells or greater than about 50% tumor-reactive T cells, or any value between any of the foregoing. In some embodiments, a provided TIL composition includes tumor reactive T cells that are present in a composition in a therapeutically effective amount such that the composition exhibits different characteristics from natural TIL population(s). In some embodiments, a provided TIL composition includes tumor reactive T cells positive for one or more marker as described herein (e.g. PD-1/CD39) that are present in a composition in a therapeutically effective amount such that the composition exhibits different characteristics from natural TIL populations. In some embodiments, a provided TIL composition comprising a therapeutically effective amount of tumor reactive T cells includes a greater percentage of tumor reactive T cells in the composition compared to natural TIL populations. In some embodiments, a provided TIL composition includes a percentage of tumor reactive T cells so that it is enriched for tumor reactive T cells compared to the amount (e.g., percent) that are present endogenously, i.e., greater than the percentage of cells that are naturally occurring in an individual with a cancer e.g., with a tumor. Such an amount can be administered to a cancer patient.

The provided TIL compositions enriched in tumor reactive cells exhibit a number of functional or phenotypic activities that evidence their reactivity to tumor cells. In some embodiments, cells can be assessed for any of a number of functional or phenotypic activities, including but not limited to cytotoxic activity, degranulation, ability to produce or secrete cytokines, and expression of one or more intracellular or surface phenotypic markers. Methods to assess such activities are known and are exemplified herein and in working examples.

In some embodiments, upon recognition of tumor cells, TILs can become activated. Upon activation, the TILs produce cytokines, chemokines and other factors abundantly and at the same time exhibit potent cytolytic activity. In some embodiments, activation triggers the release of cytoplasmic granules containing granzymes, leading to target cell death. Assays to measure cytokines, chemokines and other soluble factors are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, TILs can be evaluated for general functional activity, such as based on IFN-γ and/or granzyme B secretion or other cytokine secretion, in response to a polyclonal stimulation. In some embodiments, the polyclonal stimulation is stimulation of CD3 (e.g., with OKT3). In some embodiments, the in vitro CD3 assay includes OKT3 stimulation. In some embodiments, the in vitro CD3 assay includes washing and seeding TIL into culture plates precoated with OKT3 diluted in phosphate-buffered saline. In some embodiments, the polyclonal stimulation is stimulation of CD3 (e.g. with OKT3) and CD28 to provide a costimulatory signal. In some embodiments, the in vitro assay includes stimulation with an anti-CD3 and anti-CD28 antibody, such as by incubation of cells with Dynabeads. After overnight incubation, the supernatants are harvested and protein in the supernatant is measured by ELISA for cytokines of interest.

In some embodiments, the provided TIL compositions are assessed for tumor or neoantigen reactivity, for example by an in vitro assay. In some embodiments, the assay can be an in vitro autologous tumor assay. In some embodiments, the assay is an in vitro co-culture assay. In these and related embodiments, the results from such assays (e.g., an in vitro autologous tumor assay or in vitro co-culture assay or like assay) can be used as criteria to characterize the TIL compositions and/or cell populations making up the composition. Such criteria can include without limitation the presence of and/or amounts or levels of one more of the following: cytotoxic activity (e.g., tumor cell killing), cell activation and/or reactivity (e.g., against tumor cells) production and/or secretion of one more of cytokines (e.g., IFN-γ and/or granzyme B secretion) or production or secretion of other compound related to one or more of cytotoxic activity, cell activation, cell reactivity, cell viability or cell exhaustion.

In some embodiments, TILs can be evaluated for cytokine secretion, e.g. IFN-γ and/or granzyme B secretion, in response to co-culture with autologous tumor digest in an in vitro autologous tumor assay. In some embodiments, reference to an in vitro autologous tumor assay is understood to be an assay in which TIL are incubated with non-hematopoietic cells from an autologous primary tumor. In some embodiments, the in vitro autologous tumor assay includes seeding TILs into a culture plate with autologous non-hematopoietic tumor cells (e.g., 1:1 ratio). In some embodiments, the autologous tumor cells are single cell suspensions of CD45 negative (CD45-) cells obtained from a primary tumor. After a period of incubation ranging from 12-24 hours, supernatants are harvested and factor release can be quantified, for example by ELISA.

In some embodiments, TILs can be evaluated for cytokine secretion, e.g. IFN-γ and/or granzyme B secretion, in response to co-culture with APCs loaded with neoantigen (i.e., neoantigenic peptides) in an in vitro co-culture assay. In some embodiments, reference to an in vitro co-culture assay is understood to be an assay in which TIL are incubated with autologous APCs loaded with autologous neoantigenic peptides (hereinafter also referred to as peptide loaded autologous APCs). In some embodiments, the in vitro co-culture assay includes seeding TILs into a culture plate with autologous irradiated APCs presenting neoantigenic peptide. In some embodiments, the APCs are irradiated. IN some embodiments, the APCs are blood-derived APCs, such as B cells or dendritic cells. In some embodiments, the in vitro co-culture assay referenced herein is an assay in which B cells are isolated and expanded from autologous blood or apheresis, such as by culture with CD40L and IL-4 for 14 days before loading with neoantigenic peptide, and then co-culture with TIL at a ratio ranging from 1:1 to 1:5 TIL:APC. After a period of incubation ranging from 12-24 hours, supernatants are harvested and factor release can be quantified, for example by ELISA. In some aspects, an in vitro co-culture assay results in strong T cell activation. Without wishing to be bound by theory, it is considered that the APCs present in the in vitro co-culture assay express robust levels of HLA and costimulatory molecules required to optimally activate T cells, in addition to being pulsed with neoantigenic peptide cognate to the TIL TCR.

In some embodiments, the provided TIL composition includes an increased or greater percentage of cells that exhibit neoantigen reactivity compared to an unselected TIL composition. In some embodiments, the neoantigen reactivity is increased by greater than 2-fold, greater than 3-fold, greater than 4-fold, greater than 5-fold, greater than 6-fold, greater than 7-fold, greater than 8-fold, greater than 9-fold, greater than 10-fold, greater than 15-fold, greater than 20-fold, greater than 30-fold, greater than 40-fold, greater than 50-fold or more.

In some embodiments, the provided TIL compositions display higher neoantigen reactivity than a selected TIL population or TILs in a neoantigen reactivity assay, such as an in vitro co-culture assay or an in vitro autologous tumor assay. In some embodiments, among CD8+ T cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, among CD4+ T cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, among total T cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, among total cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, the TIL composition exhibits greater than about 2-fold more neoantigen reactivity, 3-fold more neoantigen reactivity, 4-fold more neoantigen reactivity, or 5-fold more neoantigen reactivity in an in vitro autologous tumor assay, compared to an unselected TIL composition.

In some embodiments, among CD8+ T cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, among CD4+ T cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, among total T cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, among total cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, the TIL composition exhibits greater than about 2-fold more neoantigen reactivity, 3-fold more neoantigen reactivity, 4-fold more neoantigen reactivity, or 5-fold more neoantigen reactivity in an in vitro co-culture assay, e.g., following culture with autologous APCs (e.g. DCs or B cells) presenting neoantigen peptides, compared to an unselected TIL composition.

In some embodiments, the provided TIL compositions display higher effector cytokine responses than an unselected TIL composition. In some embodiments, among CD8+ T cells in a provided TIL composition, greater than at or about 15%, greater than at or about 20%, greater than at or about 30%, greater than at or about 40% or greater than at or about 50% produce IFN-γ, in an assay for neoantigen reactivity such as in an in vitro autologous tumor assay. In some embodiments, among CD4+ T cells in a provided TIL composition, greater than at or about 15%, greater than at or about 20%, greater than at or about 30%, greater than at or about 40% or greater than at or about 50% produce IFN-γ, in a neoantigen reactivity assay such an in vitro autologous tumor assay. In some embodiments, among total T cells in a provided TIL composition, greater than at or about 15%, greater than at or about 20%, greater than at or about 30%, greater than at or about 40% or greater than at or about 50% produce IFN-γ, in a neoantigen reactivity assay, such as fin an in vitro autologous tumor assay. In some of any of the provided embodiments, the neoantigen reactivity assay is an in vitro co-culture assay. In some of any of the provided embodiments, the neoantigen reactivity assay is an in vitro autologous tumor assay.

In some embodiments, the TIL composition produces greater than about 20-fold more IFN-γ in an in vitro autologous tumor assay compared to an unselected TIL composition. In some embodiments, the TIL composition produces greater than about 30-fold more IFN-γ, 40-fold more IFN-γ, 50-fold more IFN-γ, 60-fold more IFN-γ, 70-fold more IFN-γ, 80-fold more IFN-γ, 90-fold more IFN-γ or 100-fold more IFN-γ in an in vitro autologous tumor assay compared to an unselected TIL composition. In some embodiments, the TIL composition produces greater than about 300-fold more IFN-γ, 400-fold more IFN-γ, 500-fold more IFN-γ, 600-fold more IFN-γ, 700-fold more IFN-γ, 800-fold more IFN-γ, 900-fold more IFN-γ or 1000-fold more IFN-γ in an in vitro autologous tumor assay compared to an unselected TIL composition.

In some embodiments, the TIL composition produces IFN-γ following in an in vitro autologous tumor assay. In some embodiments, the TIL composition produces between 2,000-15,000 pg/mL, between 2,000-10,000 pg/mL, or between 2,000-5000 pg/mL IFN-γ. In some embodiments, the TIL composition produces between 3,000-15,000 pg/mL, between 3,000-10,000 pg/mL, or between 3,000-5000 pg/mL IFN-γ. In some embodiments, the TIL composition produces between 5,000-15,000 pg/mL, between 5,000-10,000 pg/mL, or between 5,000-5000 pg/mL IFN-γ. In some embodiments, the TIL composition produces 2,000 pg/mL, 5,000 pg/mL, 10,000 pg/mL, or 15,000 pg/mL IFN-γ. In some embodiments, the TIL composition produces 2,000 pg/mL IFN-γ. In some embodiments, the TIL composition produces 5,000 pg/mL IFN-γ. In some embodiments, the TIL composition produces 10,000 pg/mL IFN-γ. In some embodiments, the TIL composition produces 15,000 pg/mL IFN-γ.

In some embodiments, the TIL composition produces IFN-γ following an in vitro co-culture assay, e.g., culture with autologous APCs (e.g. DCs or B cells) presenting neoantigen peptides. In some embodiments, the TIL composition produces between 2,000-1,500,000 pg/mL, between 2,000-1,000,000 pg/mL, between 2,000-500,000 pg/mL, or between 2,000-250,000 pg/mL IFN-γ. In some embodiments, the TIL composition produces between 5,000-1,500,000 pg/mL, between 5,000-1,000,000 pg/mL, between 5,000-500,000 pg/mL, or between 5,000-250,000 pg/mL IFN-γ. In some embodiments, the TIL composition produces between 5,000-1,500,000 pg/mL, between 100,000-1,000,000 pg/mL, between 100,000-500,000 pg/mL, or between 100,000-250,000 pg/mL IFN-γ. In some embodiments, the TIL composition produces 5,000 pg/mL, 10,000 pg/mL, 20,000 pg/mL, 30,000 pg/mL, 40,000 pg/mL, or 50,000 pg/mL IFN-γ. In some embodiments, the TIL composition produces 50,000 pg/mL, 100,000 pg/mL, 200,000 pg/mL, 300,000 pg/mL, 400,000 pg/mL, 500,000 pg/mL, 600,000 pg/mL, 700,000 pg/mL, 800,000 pg/mL, 900,000 pg/mL, 1,000,000 pg/mL, or 1,500,000 pg/mL IFN-γ. In some embodiments, the TIL composition produces 2,000 pg/mL IFN-γ. In some embodiments, the TIL composition produces 5,000 pg/mL IFN-γ. In some embodiments, the TIL composition produces 50,000 pg/mL IFN-γ. In some embodiments, the TIL composition produces 500,000 pg/mL IFN-γ. In some embodiments, the TIL composition produces 600,000 pg/mL IFN-γ. In some embodiments, the TIL composition produces 700,000 pg/mL IFN-γ. In some embodiments, the TIL composition produces 800,000 pg/mL IFN-γ. In some embodiments, the TIL composition produces 900,000 pg/mL IFN-γ. In some embodiments, the TIL composition produces 1,000,000 pg/mL IFN-γ. In some embodiments, the TIL composition produces 1,500,000 pg/mL IFN-γ.

In some embodiments, among CD8+ T cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides. In some embodiments, among CD4+ T cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides. In some embodiments, among total T cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides. In some embodiments, among total cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides.

In some embodiments, among CD8+ T cells in a provided TIL composition, greater than at or about 15%, greater than at or about 20%, greater than at or about 30%, greater than at or about 40% or greater than at or about 50% produce TNF-α in an in vitro autologous tumor assay. In some embodiments, among CD4+ T cells in a provided TIL composition, greater than at or about 15%, greater than at or about 20%, greater than at or about 30%, greater than at or about 40% or greater than at or about 50% produce TNF-α in an in vitro autologous tumor assay. In some embodiments, among total T cells in a provided TIL composition, greater than at or about 15%, greater than at or about 20%, greater than at or about 30%, greater than at or about 40% or greater than at or about 50% produce TNF-α in an in vitro autologous tumor assay. In some embodiments, among total cells in a provided TIL composition, greater than at or about 15%, greater than at or about 20%, greater than at or about 30%, greater than at or about 40% or greater than at or about 50% produce TNF-α in an in vitro autologous tumor assay.

In some embodiments, the TIL composition produces greater than about 50-fold more TNF-α following an in vitro co-culture assay, e.g., culture with autologous APCs (e.g. DCs or B cells) presenting neoantigen peptides, compared to an unselected TIL composition. In some embodiments, the TIL composition produces greater than about 5-fold more TNF-α, 10-fold more TNF-α, 15-fold more TNF-α, 20-fold more TNF-α, 25-fold more TNF-α, 30-fold more TNF-α, 35-fold more TNF-α or 40-fold more TNF-α in an in vitro co-culture assay e.g., following culture with autologous APCs (e.g. DCs or B cells) presenting neoantigen peptides, compared to an unselected TIL composition. In some embodiments, the TIL composition produces greater than about 75-fold more TNF-α, 100-fold more TNF-α, 150-fold more TNF-α, 200-fold more TNF-α, 250-fold more TNF-α, 300-fold more TNF-α, 350-fold more TNF-α or 400-fold more TNF-α in an in vitro co-culture assay e.g., following culture with autologous APCs (e.g. DCs or B cells) presenting neoantigen peptides, compared to an unselected TIL composition.

In some embodiments, the TIL composition produces greater than about 50-fold more TNF-α in an in vitro autologous tumor assay compared to an unselected TIL composition. In some embodiments, the TIL composition produces greater than about 5-fold more TNF-α, 10-fold more TNF-α, 15-fold more TNF-α, 20-fold more TNF-α, 25-fold more TNF-α, 30-fold more TNF-α, 35-fold more TNF-α or 40-fold more TNF-α in an in vitro autologous tumor assay compared to an unselected TIL composition. In some embodiments, the TIL composition produces greater than about 75-fold more TNF-α, 100-fold more TNF-a, 150-fold more TNF-α, 200-fold more TNF-α, 250-fold more TNF-α, 300-fold more TNF-a, 350-fold more TNF-α or 400-fold more TNF-α in an in vitro autologous tumor assay compared to an unselected TIL composition.

In some embodiments, the TIL composition produces TNF-α in an in vitro co-culture assay, e.g., culture with autologous APCs (e.g. DCs or B cells) presenting neoantigen peptides. In some embodiments, the TIL composition produces between 250-2500 pg/mL, between 250-2000 pg/mL, between 250-1500 pg/mL, between 250-1000 pg/mL, between 250-500 pg/mL TNF-α. In some embodiments, the TIL composition produces 250 pg/mL, 500 pg/mL, 1000 pg/mL, 1500 pg/mL, 2000 pg/mL, or 2500 pg/mL, TNF-α. In some embodiments, the TIL composition produces 250 pg/mL TNF-α. In some embodiments, the TIL composition produces 500 pg/mL TNF-α. In some embodiments, the TIL composition produces 1000 pg/mL TNF-α. In some embodiments, the TIL composition produces 1500 pg/mL TNF-α. In some embodiments, the TIL composition produces 2000 pg/mL TNF-α. In some embodiments, the TIL composition produces 2500 pg/mL TNF-α.

In some embodiments, the cytotoxic activity can be determined based on the ability to produce or secrete granzyme B in a neoantigen reactivity assays, such as an in vitro co-culture assay or an in vitro autologous tumor assay. In some embodiments, the TIL composition produces greater than about 10-fold more granzyme B in an in vitro co-culture assay, e.g. following culture with autologous APCs (e.g. DCs or B cells) presenting neoantigen peptides, compared to an unselected TIL composition. In some embodiments, the TIL composition produces greater than about 20-fold more granzyme B, 30-fold more granzyme B, 40-fold more granzyme B or 50-fold more granzyme B in an in vitro co-culture assay, e.g., following culture with autologous APCs (e.g. DCs or B cells) presenting neoantigen peptides, compared to an unselected TIL composition. In some embodiments, the TIL composition produces greater than about 100-fold more granzyme B in an in vitro co-culture assay, e.g. following culture with autologous APCs (e.g. DCs or B cells) presenting neoantigen peptides, compared to an unselected TIL composition. In some embodiments, the TIL composition produces greater than about 200-fold more granzyme B, 300-fold more granzyme B, 400-fold more granzyme B or 500-fold more granzyme B in an in vitro co-culture assay, e.g., following culture with autologous APCs (e.g. DCs or B cells) presenting neoantigen peptides, compared an unselected TIL composition. In some embodiments, the TIL composition produces greater than about 1000-fold more granzyme B in an in vitro co-culture assay, e.g. following culture with autologous APCs (e.g. DCs or B cells) presenting neoantigen peptides, compared to an unselected TIL composition. In some embodiments, the TIL composition produces greater than about 2000-fold more granzyme B, 3000-fold more granzyme B, 4000-fold more granzyme B or 5000-fold more granzyme B in an in vitro co-culture assay, e.g., following culture with autologous APCs (e.g. DCs or B cells) presenting neoantigen peptides, compared to an unselected TIL composition. In some embodiments, the TIL composition produces greater than about 10,000-fold more granzyme B in an in vitro co-culture assay, e.g. following culture with autologous APCs (e.g. DCs or B cells) presenting neoantigen peptides, compared to an unselected TIL composition.

In some embodiments, the TIL composition produces greater than about 10-fold more granzyme B in an in vitro autologous tumor assay compared to an unselected TIL composition. In some embodiments, the TIL composition produces greater than about 20-fold more granzyme B, 30-fold more granzyme B, 40-fold more granzyme B or 50-fold more granzyme B in an in vitro autologous tumor assay compared to an unselected TIL composition. In some embodiments, the TIL composition produces greater than about 100-fold more granzyme B in an in vitro autologous tumor assay compared to an unselected TIL composition. In some embodiments, the TIL composition produces greater than about 200-fold more granzyme B, 300-fold more granzyme B, 400-fold more granzyme B or 500-fold more granzyme B in an in vitro autologous tumor assay compared to an unselected TIL composition. In some embodiments, the TIL composition produces greater than about 1000-fold more granzyme B in an in vitro autologous tumor assay compared to an unselected TIL composition. In some embodiments, the TIL composition produces greater than about 2000-fold more granzyme B, 3000-fold more granzyme B, 4000-fold more granzyme B or 5000-fold more granzyme B in an in vitro autologous tumor assay compared to an unselected TIL composition. In some embodiments, the TIL composition produces greater than about 10,000-fold more granzyme B in an in vitro autologous tumor assay compared to an unselected TIL composition.

In some embodiments, the TIL composition produces granzyme B in an autologous tumor assay. In some embodiments, the TIL composition produces between 200-3,000 pg/mL, between 200-1,000 pg/mL, or between 200-500 pg/mL granzyme B. In some embodiments, the TIL composition produces between 300-3,000 pg/mL, between 300-1,000 pg/mL, or between 300-500 pg/mL granzyme B. In some embodiments, the TIL composition produces 200 pg/mL, 500 pg/mL, 1,000 pg/mL, or 3,000 pg/mL granzyme B. In some embodiments, the TIL composition produces 1,000 pg/mL granzyme B. In some embodiments, the TIL composition produces 3,000 pg/mL granzyme B.

In some embodiments, the TIL composition produces granzyme B in an in vitro co-culture assay, e.g., culture with autologous APCs (e.g. DCs or B cells) presenting neoantigen peptides. In some embodiments, the TIL composition produces between 50,000-600,000 pg/mL, between 50,000-500,000 pg/mL, between 50,000-400,000 pg/mL, between 50,000-300,00 pg/mL, between 200,00-500 pg/mL granzyme B, between 000,00-500 pg/mL granzyme B. In some embodiments, the TIL composition produces 50,000 pg/mL, 100,000 pg/mL, 200,000 pg/mL, 300,000 pg/mL, 400,000 pg/mL, 500,000 pg/mL or 600,000 pg/mL, granzyme B. In some embodiments, the TIL composition produces 50,000 pg/mL granzyme B. In some embodiments, the TIL composition produces 100,000 pg/mL granzyme B. In some embodiments, the TIL composition produces 200,000 pg/mL granzyme B. In some embodiments, the TIL composition produces 300,000 pg/mL granzyme B. In some embodiments, the TIL composition produces 400,000 pg/mL granzyme B. In some embodiments, the TIL composition produces 500,000 pg/mL granzyme B. In some embodiments, the TIL composition produces 600,000 pg/mL granzyme B.

In some embodiments, the provided TIL compositions display higher degranulation responses in a neoantigen reactivity assay, such as in in vitro co-culture assay or an in vitro autologous tumor assay, than an unselected TIL composition. In some embodiments, among CD8+ T cells in a provided TIL composition, greater than at or about 15%, greater than at or about 20%, greater than at or about 30%, greater than at or about 40% or greater than at or about 50% exhibit degranulation in an in vitro co-culture assay e.g., following culture with autologous APCs (e.g. DCs or B cells) presenting neoantigen peptides. In some embodiments, degranulation activity can be measured by CD107a expression. In some embodiments, among CD8+ T cells in a provided TIL composition, greater than at or about 15%, greater than at or about 20%, greater than at or about 30%, greater than at or about 40% or greater than at or about 50% express CD107a in an in vitro co-culture assay, e.g., following culture with autologous APCs (e.g. DCs or B cells) presenting neoantigen peptides. In some embodiments, among CD8+ T cells in a provided TIL composition, greater than at or about 10% express CD107a in an in vitro co-culture assay, e.g., following culture with autologous APCs (e.g. DCs or B cells) presenting neoantigen peptides. In some embodiments, among CD8+ T cells in a provided TIL composition, greater than at or about 20% express CD107a in an in vitro co-culture assay, e.g., following culture with autologous APCs (e.g. DCs or B cells) presenting neoantigen peptides. In some embodiments, among CD8+ T cells in a provided TIL composition, greater than at or about 25% express CD107a in an in vitro co-culture assay, e.g., following culture with autologous APCs (e.g. DCs or B cells) presenting neoantigen peptides.

In some embodiments, among CD4+ T cells in a provided TIL composition, greater than at or about 15%, greater than at or about 20%, greater than at or about 30%, greater than at or about 40% or greater than at or about 50% exhibit degranulation in an in vitro co-culture assay, e.g., following culture with autologous APCs (e.g. DCs or B cells) presenting neoantigen peptides. In some embodiments, degranulation activity can be measured by CD107a expression. In some embodiments, among CD4+ T cells in a provided TIL composition, greater than at or about 15%, greater than at or about 20%, greater than at or about 30%, greater than at or about 40% or greater than at or about 50% express CD107a in an in vitro co-culture assay, e.g., following culture with autologous APCs (e.g. DCs or B cells) presenting neoantigen peptides. In some embodiments, among CD4+ T cells in a provided TIL composition, greater than at or about 5% express CD107a in an in vitro co-culture assay, e.g., following culture with autologous APCs (e.g. DCs or B cells) presenting neoantigen peptides. In some embodiments, among CD4+ T cells in a provided TIL composition, greater than at or about 10% express CD107a in an in vitro co-culture assay e.g., following culture with autologous APCs (e.g. DCs or B cells) presenting neoantigen peptides. In some embodiments, among CD8+ T cells in a provided TIL composition, greater than at or about 15% express CD107a in an in vitro co-culture assay, e.g., following culture with autologous APCs (e.g. DCs or B cells) presenting neoantigen peptides.

In some embodiments, among CD8+ T cells in a provided TIL composition, greater than at or about 15%, greater than at or about 20%, greater than at or about 30%, greater than at or about 40% or greater than at or about 50% exhibit degranulation in an in vitro autologous tumor assay. In some embodiments, degranulation activity can be measured by CD107a expression. In some embodiments, among CD8+ T cells in a provided TIL composition, greater than at or about 15%, greater than at or about 20%, greater than at or about 30%, greater than at or about 40% or greater than at or about 50% express CD107a in an in vitro autologous tumor assay. In some embodiments, among CD8+ T cells in a provided TIL composition, greater than at or about 10% express CD107a in an in vitro autologous tumor assay. In some embodiments, among CD8+ T cells in a provided TIL composition, greater than at or about 20% express CD107a in an in vitro autologous tumor assay. In some embodiments, among CD8+ T cells in a provided TIL composition, greater than at or about 25% express CD107a in an in vitro autologous tumor assay.

In some embodiments, among CD4+ T cells in a provided TIL composition, greater than at or about 15%, greater than at or about 20%, greater than at or about 30%, greater than at or about 40% or greater than at or about 50% exhibit degranulation in an in vitro autologous tumor assay. In some embodiments, degranulation activity can be measured by CD107a expression. In some embodiments, among CD4+ T cells in a provided TIL composition, greater than at or about 15%, greater than at or about 20%, greater than at or about 30%, greater than at or about 40% or greater than at or about 50% express CD107a in an in vitro autologous tumor assay. In some embodiments, among CD4+ T cells in a provided TIL composition, greater than at or about 5% express CD107a in an in vitro autologous tumor assay. In some embodiments, among CD4+ T cells in a provided TIL composition, greater than at or about 10% express CD107a in an in vitro autologous tumor assay. In some embodiments, among CD8+ T cells in a provided TIL composition, greater than at or about 15% express CD107a in an in vitro autologous tumor assay.

In some embodiments, the TIL composition is characterized by its ability to kill tumor cells in an in vitro autologous tumor assay. In some embodiments, the TIL composition kills at least 30% of tumor cells in an in vitro autologous tumor assay. In some embodiments, the TIL composition kills at least 40% of tumor cells in an in vitro autologous tumor assay. In some embodiments, the TIL composition kills at least 50% of tumor cells in an in vitro autologous tumor assay. In some embodiments, the TIL composition kills at least 60% of tumor cells in an in vitro autologous tumor assay. In some embodiments, the TIL composition kills at least 70% of tumor cells in an in vitro autologous tumor assay. In some embodiments, the TIL composition kills at least 80% of tumor cells in an in vitro autologous tumor assay.

In certain embodiments, the number of such cells in the composition is a therapeutically effective amount. An effective amount of cells can vary depending on the patient, as well as the type, severity and extent of disease. Thus, a physician can determine what an effective amount is after considering the health of the subject, the extent and severity of disease, and other variables. In some embodiments, the amount is an amount that reduces the severity, the duration and/or the symptoms associated with cancer in an animal. In some embodiments, a therapeutically effective amount is a dose of cells that results in a reduction of the growth or spread of cancer by at least 2.5%, at least 5%, at least 10%, at least 15%, at least 25%, at least 35%, at least 45%, at least 50%, at least 75%, at least 85%, by at least 90%, at least 95%, or at least 99% in a patient or an animal administered a composition described herein relative to the growth or spread of cancer in a patient (or an animal) or a group of patients (or animals) not administered the composition. In some embodiments, a therapeutically effective amount is an amount to result in cytotoxic activity resulting in activity to inhibit or reduce the growth of cancer cells.

In some embodiments, the TIL composition provided herein enriched in tumor reactive cells comprises an amount of cells that is from at or about 10⁵ and at or about 10¹² cells. In some embodiments, the TIL composition provided herein enriched in tumor reactive cells comprises an amount of cells from at or about 10⁵ to at or about 10⁸ cells. In some embodiments, the TIL composition provided herein enriched in tumor reactive cells comprises an amount of cells from at or about 10⁶ and at or about 10¹². In some embodiments, the TIL composition provided herein enriched in tumor reactive cells comprises an amount of cells from at or about 10⁸ and at or about 10¹¹ cells. In some embodiments, the TIL composition provided herein enriched in tumor reactive cells comprises an amount of cells from at or about 10⁹ and at or about 10¹⁰ cells. In some embodiments, the TIL composition provided herein enriched in tumor reactive cells comprises an amount of greater than or greater than at or about 10⁵ cells, greater than or greater than at or about 10⁶ cells, greater than or greater than at or about 10⁷ cells, greater than or greater than at or about 10⁸ cells, greater than or greater than at or about 10⁹ cells, greater than or greater than at or about 10¹⁰ cells, greater than or greater than at or about 10¹¹ cells, or greater than or greater than at or about 10¹² cells. In some embodiments, such an amount can be administered to a subject having a disease or condition, such as to a cancer patient.

In some embodiments, the volume of the composition is at least or at least about 10 mL, 50 mL, 100 mL, 200 mL, 300 mL, 400 mL or 500 mL, such as is from or from about 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL, each inclusive. In some embodiments, the composition has a cell density of at least or at least about 1×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the cell density of the composition is between or between about 1×10⁵ cells/mL to 1×10⁸ cells/mL, 1×10⁵ cells/mL to 1×10⁷ cells/mL, 1×10⁵ cells/mL to 1×10⁶ cells/mL, 1×10⁶ cells/mL to 1×10⁷ cells/mL, 1×10⁶ cells/mL to 1×10⁸ cells/mL, 1×10⁶ cells/mL to 1×10⁷ cells/mL or 1×10⁷ cells/mL to 1×10⁸ cells/mL, each inclusive.

Among the compositions are pharmaceutical compositions and formulations for administration, such as for adoptive cell therapy. In some embodiments, the cells are formulated with a pharmaceutically acceptable carrier. In some examples, the pharmaceutical composition comprising a pharmaceutically effective carrier is in a therapeutically effective amount sufficient to treat, ameliorate or otherwise beneficially alter the symptoms of a condition, disorder or disease or other indication (e.g., a cancer).

A pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for cells, such as a saline solution, a dextrose solution or a solution comprising human serum albumin.

In some embodiments, the pharmaceutically acceptable carrier or vehicle for such compositions is any non-toxic aqueous solution in which the cells can be maintained, or remain viable, for a time sufficient to allow administration of live cells. For example, the pharmaceutically acceptable carrier or vehicle can be a saline solution or buffered saline solution. The pharmaceutically acceptable carrier or vehicle can also include various bio materials that may increase the efficiency of cells. Cell vehicles and carriers can, for example, include polysaccharides such as methylcellulose (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P(NIPAM-co-AA) (Y. H. Bae, B. Vernon, C. K. Han, S. W. Kim, J. Control. Release 53, 249, 1998; H. Gappa, M. Baudys, J. J. Koh, S. W. Kim, Y. H. Bae, Tissue Eng. 7, 35, 2001, each of which is incorporated herein by reference in its entirety), as well as Poly(oxyethylene)/poly(D,L-lactic acid-co-glycolic acid) (B. Jeong, K. M. Lee, A. Gutowska, Y. H. An, Biomacromolecules 3, 865, 2002, which is incorporated herein by reference in its entirety), P(PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lee, Po-Han Kung and Yu-Der Lee, Carbohydrate Polymers, 61, 348, 2005, which is incorporated herein by reference in its entirety), collagen (Lee C R, Grodzinsky A J, Spector M., Biomaterials 22, 3145, 2001, which is incorporated herein by reference in its entirety), alginate (Bouhadir K H, Lee K Y, Alsberg E, Damm K L, Anderson K W, Mooney D J. Biotech Prog 17, 945, 2001; Smidsrd O, Skjak-Braek G., Trends Biotech, 8, 71, 1990, each of which is incorporated herein by reference in its entirety).

In some embodiments, the composition, including pharmaceutical composition, is sterile. In some embodiments, isolation or enrichment of the cells is carried out in a closed or sterile environment, for example, to minimize error, user handling and/or contamination. In some embodiments, sterility may be readily accomplished, e.g., by filtration through sterile filtration membranes.

Also provided herein are compositions that are suitable for cryopreserving the provided T cells, including tumor-reactive T cells. In some embodiments, the composition comprises a cryoprotectant. In some embodiments, the cryoprotectant is or comprises DMSO and/or glycerol. In some embodiments, compositions formulated for cryopreservation can be stored at low temperatures, such as ultra low temperatures, for example, storage with temperature ranges from −40° C. to −150° C., such as or about 80° C.±6.0° C.

Also provided herein is a frozen composition containing any of the provided TIL compositions and a cryoprotectant.

In some embodiments, the cryopreserved cells are prepared for administration by thawing. In some cases, the cells can be administered to a subject immediately after thawing. In such an embodiment, the composition is ready-to-use without any further processing. In other cases, the cells are further processed after thawing, such as by resuspension with a pharmaceutically acceptable carrier, incubation with an activating or stimulating agent, or are activated washed and resuspended in a pharmaceutically acceptable buffer prior to administration to a subject.

### II. Ex Vivo Methods of Producing TIL Compositions Enriched in Tumor-Reactive T Cells

Various embodiments of the provided methods involve the ex vivo expansion and production of a T cell therapeutic composition, particularly for use in connection with treating cancer. In some embodiments, the method of manufacturing involves the growth and manipulation of patient cells outside of the body.

The provided embodiments relate to processes for preparing a therapeutic TIL composition enriched for tumor reactive T cells that involves a direct selection of cells to yield a tumor reactive cell population that is further expanded. In the provided methods, a TIL tumor sample is obtained that contains or is expected to contain tumor reactive T cells (e.g. first population). In one embodiment, this population can be processed by digestion to create a single cell suspension (e.g. second population). In some embodiments, this second population is sorted to select for cells that are enriched for tumor reactive T cells and to minimize the presence of bystander cells, such as regulatory T cells. In some embodiments, the selection is for cells positive for PD-1 and/or CD39. In some embodiments, the selection is for cells positive for PD-1 and CD39. In provided methods, the T cells are sorted directly after tumor digest or after a short period of cell culture for cells surface positive for CD39, and PD1 to create a population of selected T cells. This process removes the nonreactive and inhibitory ‘bystander’ cells, resulting in T cell product enriched in neoantigen reactive T cells. The selection yields a selected or sorted population of cells (e.g. in some cases also called a third population), which then can be expanded to create a therapeutic composition containing an expanded population of tumor specific reactive cells (e.g. in some cases also called a fourth population). In some embodiments, cells are then expanded into clinically relevant numbers of tumor specific T cells. In aspects of the provided methods, the T cells from the selected population (third population), such as present from the sorted T selected cells from a resected tumor fragment or a single cell suspension therefrom, are expanded by culture in the presence of one or more T cell stimulatory agent(s) under conditions for stimulating the T cells. In some embodiments, the final expanded therapeutic composition is formulated with a cryoprotectant for cryopreservation.

### A. Sample Containing T Cells

The provided methods include selecting or obtaining an input sample of T cells from a biological sample, which can be used as the source or input of T cells for stimulation with one or more T cell stimulatory agents(s) (e.g. recombinant IL-2 or other T cell stimulating cytokines and/or anti-CD3). In some embodiments, the T cells are from a biological sample from a subject that is known or likely to contain tumor reactive T cells. In some embodiments, the biological sample provides an input population of cells that is a single cell suspension (SCS), which can be used for subsequent selection of TILs for expansion as described in Section II.B and II.C. In some embodiments, the biological sample is processed to provide an input population of cells that is a single cell suspension, which can be used for subsequent selection of TILs for expansion as described in Section II.B and II.C. For instance, particular methods include processing tumor fragments by dissociation using homogenization and/or enzymatic methods and filtering the dissociated cells to prepare a single cell suspension as an input population of cells.

In aspects of any of the provided embodiments, a suitable biological sample from a subject, such as from a patient of interest, i.e., a patient suspected of having or known to have cancer, is obtained. In some embodiments, the sample is one that is known or suspected of containing T cells, such as T cells that may be or may likely express an endogenous T cell receptor (TCR). The biological sample may be derived from any initial source that would contain or is suspected of containing such T cells. In some aspects, biological sample sources of interest include, but are not limited to, many different physiological sources, e.g. tissue derived samples, e.g. homogenates, and blood or derivatives thereof.

Any of a variety of biological samples can be used as a source of potentially reactive T cells. Although the tumor and downstream lymph nodes may have the highest frequency of reactive T cells (Powell et al., Clin. Cancer. Res., 2014), other sample sources also can be used. In some cases the sample is a tumor sample, a tertiary lymphoid site, a draining lymph node, peripheral blood or bone marrow. In some embodiments, the biological sample is a tumor sample. In some embodiments, the biological sample is a lymph sample. In some embodiments, the biological sample is a peripheral blood sample.

The biological samples include tissue, fluid, and other samples taken directly from the subject to obtain an input sample, or can undergo one or more processing steps, such as separation, e.g. selection or enrichment, centrifugation, washing, and/or incubation, to obtain or produce an input sample. The input sample containing T cells can be a sample obtained directly from a biological source or a sample that is processed. Biological samples include, but are not limited to, body fluids, such as blood, plasma, serum, cerebrospinal fluid, synovial fluid, urine and sweat, tissue and organ samples, and tumor samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In many embodiments, the sample may be derived from fluids in which the T cells of interest are at least suspected of being present. In many embodiments, a suitable initial source for the sample is blood. In some embodiments, the biological sample is a blood-derived sample. The blood-derived sample may be derived from whole blood or a fraction thereof, e.g. serum, plasma, etc., where in many embodiments the sample is derived from blood cells harvested from whole blood. In some aspects, the sample source contains mononuclear cells. For example, a biological sample is or contains peripheral blood mononuclear cells (PBMCs) or is derived from PBMCs.

In some embodiments in which the sample is a PBMC derived sample, the sample is generally a fluid PBMC derived sample. Any convenient methodology for producing a fluid PBMC sample may be employed. In many embodiments, the fluid PBMC derived sample is prepared by separating PBMCs from whole blood, i.e., collecting PBMCs, e.g., by centrifugation (such as by Ficoll-Hypaque density gradient centrifugation, where representative protocols for such separation procedures are disclosed in WO 98/15646 and U.S. Pat. No. 5,985,565).

In some embodiments, the sample is a tumor sample and thereby provides a source of tumor-infiltrating lymphocytes (TILs). In some aspects, TILs are T cells that have left the bloodstream of a subject and migrated into or infiltrated a tumor. In particular aspects, TILs are reactive to a tumor antigen.

A patient tumor sample may be obtained by any of a variety of methods in which the method obtains a sample that contains a mixture of tumor and TIL cells. In some embodiments, the tumor sample is obtained by surgical resection. In some embodiments, the tumor sample is obtained by needle biopsy. In general, the tumor sample may be from any solid tumor, including primary tumors, invasive tumors or metastatic tumors. The tumor sample may also be a liquid tumor, such as a tumor obtained from a hematological malignancy.

In some embodiments, the solid tumor may be of any cancer type, including, but not limited to, ovarian, vulva, endometrial, urothelial, breast, pancreatic, prostate, colorectal, lung, brain, renal, stomach (gastrointestinal), and skin (including but not limited to squamous cell carcinoma, basal cell carcinoma, and melanoma). In some embodiments, the tumor is from a patient with a cancer, including, but not limited to, ovarian, vulva, endometrial, urothelial, breast, colorectal, lung, renal, and skin (including but not limited to melanoma). In some embodiments, the tumor is from a patient with an ovarian cancer. In some embodiments, the tumor is from a patient with cancer of the vulvar. In some embodiments, the tumor is from a patient with an endometrial cancer. In some embodiments, the tumor is from a patient with a urothelial cancer. In some embodiments, the tumor is from a patient with a breast cancer. In some embodiments, the tumor is from a patient with a colorectal cancer. In some embodiments, the tumor is from a patient with a lung cancer. In some embodiments, the tumor is from a patient with a renal cancer. In some embodiments, the tumor is from a patient with melanoma. In particular embodiments, the tumor is from a patient to be treated as described in Section III.

In particular embodiments, a T cell population is one that includes both CD4+ and CD8+ T cells. Many cancers, including solid tumors, such as many common epithelial indications (e.g. GI), express class I and class II restricted mutations. In order for a T cell product to target such indications, e.g. common epithelial indications, it is contemplated that both CD8+ T cells to recognize class I MHC-restricted molecules and CD4+ T cells to recognize Class II MHC-restricted molecules are necessary.

The sample may be obtained from a variety of different subjects/patients/hosts. Generally such hosts are “mammals” or “mammalian,” where these terms are used broadly to describe organisms which are within the class Mammalia, including the orders carnivore (e.g., dogs and cats), Rodentia (e.g., mice, guinea pigs, and rats), and primates (e.g., humans, chimpanzees, and monkeys). In many embodiments, the hosts will be humans.

In some aspects, the subject is a human. Accordingly, the cells in some embodiments are primary cells, e.g., primary human cells. In some embodiments, the sample is autologous to a subject to be treated, such as a subject who is a patient in need of a particular therapeutic intervention, such as the adoptive cell therapy for which cells are being isolated, processed, and/or expanded in accord with the provided methods. In some embodiments, the sample is allogenic to a subject to be treated.

In provided embodiments, the obtained tumor sample is fragmented into small pieces of between at or about 1 mm³ and at or about 8 mm³ in size, such as between at or about 1 mm³ and at or about 3 mm³, between at or about 1 mm³ and at or about 4 mm³, between at or about 1 mm³ and at or about 2 mm³. In some embodiments, the tumor fragment is from about −3 mm³. In some embodiments, the tumor fragment is from about 1-3 mm³. In some embodiments, the tumor fragment is obtained by physical fragmentation, such as by dissection. In some embodiments, the tumor fragment is obtained by sharp dissection.

In some of any of the provided embodiments, the obtained tumor sample is fragmented into small pieces of between at or about 1 mm and at or about 8 mm in diameter, such as between at or about 1 mm and at or about 6 mm in diameter, between at or about 1 mm and at or about 4 mm in diameter, between at or about 1 mm and at or about 2 mm in diameter. In some embodiments, the tumor fragment is from about 3 mm in diameter. In some embodiments, the tumor fragment is from about 1-2 mm in diameter. In some embodiments, the tumor fragment is obtained by physical fragmentation, such as by dissection. In some embodiments, the tumor fragment is obtained by sharp dissection.

In some embodiments, the tumor sample is cryopreserved prior to fragmentation. In some embodiments, the tumor fragments are cryopreserved.

In some embodiments, tumor fragments are used as a source to prepare a single cell suspension for use as an input sample of T cells in the provided methods. In some embodiments, the provided methods involve obtaining cells from the tumor fragments, such as by enzymatic digestion of tumor fragments to obtain TILs. Enzymatic digestion can be carried out, in part, using a collagenase, such as a type IV collagenase or a type I/II collagenase. Collagenase is an enzyme that degrades the collagen network embedded in the extracellular matrix of the cell (Eikenes et al. Anticancer Research, 2010). The enzyme, such as a collagenase, can be present in media for the enzymatic digestion at a concentration of from at or about 1 mg/mL to at or about 5 mg/mL, such as at or about 1 mg/mL, at or about 2 mg/mL, at or about 3 mg/mL, at or about 4 mg/mL at or about 5 mg/mL, at or about 6 mg/mL, at or about 7 mg/mL, at or about 8 mg/mL or at or about 9 mg/mL, at or about 10 mg/mL or any value between any of the foregoing. In some embodiments, collagenase is present in the media at a concentration of from about 5 mg/mL to about 10 mg/mL. In some embodiments, the concentration is about 5 mg/mL. In some embodiments, the concentration is 10 mg/mL. In some embodiments, the collagenase is a type IV collagenase. In some embodiments, the collagenase is a type I/II collagenase. In some embodiments, the enzymatic digestion is with a media that includes type IV collagenase, such as from at or about 5 mg/mL to at or about 10 mg/mL. In some embodiments, the enzymatic digestion is with a media that includes type I/II collagenase, such as from at or about 5 mg/mL to at or about 10 mg/mL. In some embodiments, if a more gentle digestion is desired at or about 5 mg/mL collagenase is used. In some embodiments, if a more complete digestion is desired a higher concentration of collagenase is used, such as at or about 10 mg/mL collagenase. In some embodiments, the collagenase is a type IV collagenase. In some embodiments, the collagenase is a type I/II collagenase.

In some embodiments, enzymatic digestion can be carried out, in part, using a hyaluronidase. Hyaluronidase is a hyaluronic acid-metabolizing enzyme, subsequently enhancing cell membrane permeability (Eikenes et al. Anticancer Research, 2010). The enzyme, such as a hyaluronidase, can be present in media for the enzymatic digestion at a concentration of from at or about 5 mg/mL to at or about 10 mg/mL, such as at or about 5 mg/mL, at or about 6 mg/mL, at or about 7 mg/mL, at or about 8 mg/mL or at or about 9 mg/mL, at or about 10 mg/mL or any value between any of the foregoing. In some embodiments, the enzymatic digestion is with a media that includes type II hyaluronidase, such as from at or about 5 mg/mL to at or about 10 mg/mL. In some embodiments, if a more gentle digestion is desired at or about 5 mg/mL hyaluronidase is used. In some embodiments, if a more complete digestion is desired a higher concentration of hyaluronidase is used, such as at or about 10 mg/mL hyaluronidase.

In some embodiments, DNase is also present in the media for during the enzymatic digestion. DNase is an enzyme that degrades any free DNA released into the media as a result of the tumor fragment digestion process. The enzyme, such as a DNase I, can be present in media for the enzymatic digestion at a concentration of from at or about 5,000 units/mL to at or about 10,000 units/mL, such as at or about 5,000 units/mL, at or about 6,000 units/mL, at or about 7,000 units/mL, at or about 8,000 units/mL or at or about 9,000 units/mL, at or about 10,000 units/mL or any value between any of the foregoing. In some embodiments, the enzymatic digestion is with a media that includes DNase I, such as from at or about 5,000 units/mL to at or about 10,000 units/mL.

In some embodiments, enzymes from the Miltenyi human tumor dissociation kit can be used (e.g. Cat. O. 130-095-929; Miltenyi Biotec). The enzymatic media containing the enzyme can be a serum-free media, such as any as described. In particular embodiments, enzymatic media includes hyaluronidase and/or collagenase, e.g., Roswell Park Memorial Institute (RPMI) 1640 buffer, 2 mM glutamate (e.g. GlutaMAX), 10 mg/mL gentamicin, 10,000 units/mL of DNase I, 10 mg/mL of collagenase and 10 mg/mL of hyaluronidase).

The tumor fragment is then mechanically dissected to dissociate the TILs, e.g., using a tissue dissociator. An example of a tissue dissociator is GentleMACs™ (Miltenyi Biotec) to homogenize the tissue. Tumor digests may be produced by placing the tumor in enzymatic media and mechanically dissociating the tumor for approximately 1 minute, followed by incubation for 30 minutes at 37° C. in 5% C0₂, followed by repeated cycles of mechanical dissociation and incubation under the foregoing conditions until only small tissue pieces are present. In some embodiments, tumor digests are subjected to homogenization and enzymatic digestion by incubation in the enzyme cocktail for 15 minutes to 2 hours, such as for at or about 30 minutes to 60 minutes. In some embodiments, tumor digests are subjected to homogenization and enzymatic digestion by incubation in the enzyme cocktail for about 60 minutes. At the end of this process, if the cell suspension contains a large number of red blood cells or dead cells, a density gradient separation using FICOLL can be performed to remove these cells. In some embodiments, a single cell suspension is prepared following processing of the tumor fragments by straining the cells through a filter to remove debris, such as a 70 μm strainer. In some cases, separation can be achieved by centrifugation, in which case the cell pellet can be resuspended and strained through a e.g. 70 μm strainer to remove debris. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of obtaining TILs for use in the provided methods.

In some embodiments, a single cell suspension for use as an input sample comprises from at or about 1×10⁶ dissociated tumor cells to at or about 1000×10⁶ dissociated tumor cells, such as 1×10⁶ to 500×10⁶ dissociated tumor cells, 1×10⁶ to 100×10⁶ dissociated tumor cells, 1×10⁶ to 50×10⁶ dissociated tumor cells, 1×10⁶ to 10×10⁶ dissociated tumor cells, 10×10⁶ to 1000×10⁶ dissociated tumor cells, 10×10⁶ to 100×10⁶ dissociated tumor cells, 10×10⁶ to 500×10⁶ dissociated tumor cells, 10×10⁶ to 50×10⁶ dissociated tumor cells, 50×10⁶ to 1000×10⁶ dissociated tumor cells, 50×10⁶ to 500×10⁶ dissociated tumor cells, 50×10⁶ to 100×10⁶ dissociated tumor cells, 100×10⁶ to 1000×10⁶ dissociated tumor cells, 100×10⁶ to 500×10⁶ dissociated tumor cells, or 500×10⁶ to 1000×10⁶ dissociated tumor cells. In some embodiments, a single cell suspension for use as an input sample of T cells comprises from at or about or at least at or about 10×10⁶ dissociated tumor cells, 20×10⁶ dissociated tumor cells, 30×10⁶ dissociated tumor cells, 40×10⁶ dissociated tumor cells, 50×10⁶ dissociated tumor cells, 60×10⁶ dissociated tumor cells, 70×10⁶ dissociated tumor cells, 80×10⁶ dissociated tumor cells, 90×10⁶ dissociated tumor cells, or 100×10⁶ dissociated tumor cells In some embodiments, a single cell suspension for use as an input sample of T cells comprises from at or about 10×10⁶ dissociated tumor cells to at or about 100×10⁶ dissociated tumor cells.

### B. Selection of Cells

In embodiments of the provided methods, the methods involve selecting or enriching from the first population of T cells (e.g. dissociated tumor cells), cells that are likely or suspected of being tumor reactive T cells by selecting or isolating T cells that are surface positive for the exhaustion markers PD-1 and CD39 among a lymphocyte subset of cells. In some embodiments, T cells that are positive for PD-1 and CD39 are selected or enriched from a first population of T cells that have been obtained from a biological sample, such as described in Section II.A. In particular embodiments, the selection is from cells present in the single cell suspension dissociated tumor cell sample. In some embodiments, the enriched or selected population of cells is used in subsequent processing steps, such as subsequent processing steps involving expansion in accord with the provided methods.

Methods for selection of surface receptors on cells can be by any of a number of techniques, such as generally involving antibody binding with an antibody specific reagent and subsequent enrichment by magnetic separation or fluorescence-activated cell sorting (FACS).

In some embodiments, cells are selected directly from a single cell suspension input sample prepared by enzymatic or mechanical digestion of tumor fragments, in which the selection is carried out using a CD39/PD1 positive selection. In some embodiments, the selected cells are then stimulated for expansion using methods as described, such as by incubation or culture in the presence of one or more of IL-2, IL-7, IL-15 or IL-21. In some embodiments, the stimulation would not include culture with an anti-CD3 antibody (OKT3) or other costimulatory molecules. In some embodiments, the stimulation may include culture with an anti-CD3 antibody (OKT3) or other costimulatory molecules.

In some embodiments, selection is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker*) at a relatively higher level (markerhigh) on the positively or negatively selected cells, respectively.

In particular embodiments, a T cell population is one that includes both CD4+ and CD8+ T cells. Many cancers, including solid tumors, such as many common epithelial indications (e.g. GI), express class I and class II restricted mutations. In order for a T cell product to target such indications, e.g. common epithelial indications, it is contemplated that both CD8+ T cells to recognize class I MHC-restricted molecules and CD4+ T cells to recognize Class II MHC-restricted molecules are necessary.

In some embodiments, the methods include selection of PD-1+ and CD39+ cells from among a lymphocyte subject of cells that includes CD4+ and CD8+ T cells. In one method, lymphocytes can be selected by positive selection for CD3+ cells. In another method, lymphocytes can be selected by positive selection for CD4+ and/or CD8+ cells. In some aspects, a CD4⁺ or CD8⁺ selection step, such as positive selection for CD4 and positive selection for CD8, is used to separate CD4⁺ helper and CD8⁺ cytotoxic T cells. Such selections in some aspects are carried out simultaneously and in other aspects are carried out sequentially, in either order. In some embodiments, the methods include enriching for CD4+ and CD8+ T cells by selecting for T cells surface positive for CD3 or by sequential or simultaneous selection for T cells surface positive for CD4 and T cells surface positive for CD8. Such CD3+ T cells, or CD4⁺ and/or CD8⁺ populations, can be further sorted into sub-populations by positive or negative selection for markers expressed or expressed to a relatively higher degree on tumor-reactive T cells or on T cells having expression of T cell markers associated with tumor-reactive T cells, e.g. as described above.

In some embodiments, the selections produce an enriched population of cells, such as a population of cells enriched for CD3+ T cells or CD4+ cells and CD8+ cells, that are further positive for PD-1 and CD39. In some embodiments, such cells include or are enriched for tumor-reactive T cells or T cells associated with tumor-reactive T cells. In embodiments, the selections produce an enriched population of cells, such as a population of cells enriched for CD3+ T cells or CD4+ cells and CD8+ cells, that are further positive for PD-1 and CD39, that are enriched compared to the population of cells that are isolated from a biological sample from a patient (e.g., a cancer patient).

In some embodiments, enriching for a T cell that is surface positive for one or more cell surface marker includes any method for separation based on such markers. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Methods of selection of cells include, but are not limited to, bead selection (e.g. serial bead passage for positive/negative selection of cells), immunoaffinity chromatography (e.g. serial elution for positive/negative selection, and flow cytometry sorting. For use in accord with the provided methods, the selection method meets GMP standards.

The incubation generally is carried out under conditions whereby the antibodies or binding partners, or molecules, such as secondary antibodies or other reagents, which specifically bind to such antibodies or binding partners, which are attached to the magnetic particle or bead and/or are detectably labeled, specifically bind to cell surface molecules if present on cells within the sample. In some aspects, cells bound to the antibodies can be recovered or separated from non-bound cells in the sample.

In some aspects, a combination of positive and negative selection is performed during the same selection step, where the positive and negative fractions are retained and further processed or subject to further separation steps. Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the T cells for use in connection with the provided methods can be enriched or sorted a variety of ways including, but not limited to, magnetic bead separation, fluorescent cell sorting, and disposable closed cartridge based cell sorters. In particular aspects, one or more reagents specific to T cells or a subset thereof, such as reagents specific to T cell activation markers for selecting reactive cells, can be used including, but not limited to, florescent antibodies, nanoparticles or beads on cell selection equipment, but not limited to, the CliniMACS, Sony FX500 or the Tyto cell sorting systems (Miltenyi).

In particular embodiments, selection of cells is carried out by flow cytometry-based cell sorting. Compared to other methods, flow cytometry-based cell sorting has the advantage that cells can be isolated in a single step on the basis of multiple parameters for each cell, thereby achieving a higher yield of cells and a higher purity that may not be possible with bead-based (e.g. magnetic bead-based) separations. Further, multiparameter cell staining and separation allows simultaneous labeling and identification and sorting of a plurality of antigens and characteristic fluorescent signals. Using flow cytometry sorting, a single process can remove and isolate specific populations based on a complex cell surface phenotype. Cell selection sorting equipment can be used that has a sufficiently high-throughput to handle large volumes and cell numbers. Non-limiting cell sorting equipment includes, for example, Sony FX500 or the Tyto cell sorting systems (Miltenyi). For use in provided methods, the flow cytometer instrument is GMP compliant. Method of cell sorting to achieve multiparameter sorting for two or more cell surface markers (e.g. CD39 and PD-1) can be carried out using multicolor fluorophore reagents that are compatible. It is within the level of a skilled artisan to choose appropriate fluorophores and reagents, such as by choosing a bright fluorophore and choosing fluorophores that have minimal to no spectral overlap.

In certain embodiments, the sample is contacted with a binding agent, e.g., a detectably labeled binding agent, that specifically binds to a cell surface marker. In certain embodiments, the detectably labeled binding agent(s) are fluorescently labeled. In certain embodiments, T cells labeled with binding agents specific to a cell surface marker are identified by flow cytometry. In certain embodiments, the method further includes separating any resultant T cells labeled with the binding agent(s) from other components of the sample to produce a composition enriched for T cells surface positive for the one or more cell surface marker. Cell selection sorting equipment can be used that has a sufficiently high-throughput to handle large volumes and cell numbers. Non-limiting cell sorting equipment includes, for example, Sony FX500 or the Tyto cell sorting systems (Miltenyi).

In some embodiments, any antibody reagent used to select cells in accord with the provided methods is a GMP antibody reagent. In some embodiments, the reagent is an analyte specific reagent (ASR).

In some embodiments, cells are selected that are surface positive for CD39 and PD-1. In some cases, staining methods also can include selecting CD45, CD4 and/or CD8 T cells from a sample. In some embodiments, multiparameter flow cytometry is carried out. In some embodiments, a multiparameter flow cytometry involves a sequential gating strategy. In some cases, CD45 expression, which optionally can be coupled with side scatter, can be used to exclude CD45 negative cells such as red blood cells. In some embodiments, only lymphocytes are gated based on CD45 expression and their scattering (e.g. FSClow, SSClow). In some embodiments, T cells can be gated based on positive expression of CD4+ and CD8+ cells. Within the T cell population, cells positive for CD39 and PD-1 can be identified for sorting.

Methods and antibody reagents to select for cells positive for these markers are known and commercially available. Any of a variety of fluorophores can be coupled to the antibodies and used in the multiparameter flow cytometry. In some embodiments, multi-color staining or labeling is carried out using multiple fluorophores in which multiple staining reagents against different cell surface markers are incubated with cells. In some embodiments, the fluorescent marker, e.g., conjugated to the one or more staining reagents, such as antibodies, are selected to minimize energy transfer between them, such as to avoid or minimize overlapping emission and absorption spectra. In some embodiments, each fluorescent marker has a different emission spectra. In some embodiments, the multiple fluorescent marker may be excited with a single wavelength or multiple wavelengths, but detection occurs in regions where the peak emission spectra do not overlap. In some embodiments, excitation of one or more of the fluorescent markers may be by light at a single or the same wavelength, but whereby different wavelengths of light are emitted therefrom.

In some embodiments, any fluorescent marker or fluorophore suitable for use with flow cytometry analysis can be used. Some non-limiting examples of fluorescent markers include fluorescent proteins (e.g., GFP, YFP, RFP), fluorescent moieties (e.g., fluorescein isothiocyanate) (FITC), Phycoerythrin (PE), allophycocyanin (APC), Alexa Fluor (AF)), nucleic acid colorants (e.g., 4′, 6-diamidino-2-phenylindole (DAPI), SYT016, propidium iodide (PI), cell membrane stain (e.g., FMI-43), cell functional dyes (e.g., Fluo-4, Indo-1), and synthetic dyes (e.g., Brilliant Violet (BV)). Exemplary fluorophores include, but are not limited to, hydroxycoumarin, Cascade Blue, Dylight 405 Pacific Orange, Alexa Fluor 430, Fluorescein, Oregon Green, Alexa Fluor 488, BODIPY 493, 2,7-Diochlorofluorescien, ATTO 488, Chromeo 488, Dylight 488, HiLyte 488, Alexa Fluor 532, Alexa Fluor 555, ATTO 550, BODIPY TMR-X, CF 555, Chromeo 546, Cy3, TMR, TRITC, Dy547, Dy548, Dy549, HiLyte 555, Dylight 550, BODIPY 564, Alexa Fluor 568, Alexa Fluor 594, Rhodamine, Texas Red, Alexa Fluor 610, Alexa Fluor 633, Dylight 633, Alexa Fluor 647, APC, ATTO 655, CF633, CF640R, Chromeo642, Cy5, Dylight 650, Alexa Fluor 680, IRDye 680, Alexa Fluor 700 (AF700), Cy5.5, ICG, Alexa Fluor 750, Dylight 755, IRDye 750, Cy7, PE-Cy7, Cy7.5, Alexa Fluor 790, Dylight 800, IRDye 800, BV421, BV510, BV570, BV605, BV650, BV711, BV750, BV785, Qdot® 525, Qdot® 565, Qdot® 605, Qdot® 655, Qdot® 705, or Qdot® 800. In some embodiments, the one or more fluorescent markers each individually comprise a fluorophore selected from the group consisting of PE-Cy7, APC, AF700, BV421, Aqua, and BV605.

Table 1 lists exemplary antibodies for use in staining and selection or sorting of cells as described herein.

TABLE 1
Exemplary Staining Antibodies

| | | | | |
|---|---|---|---|---|
| Target | Name | Product No. | Manufacturer | Clone |

CD39

| | | | | |
|---|---|---|---|---|
| CD39 | Anti-CD39 Mouse | 328210 | BioLegend | A1 |
| | Monoclonal Antibody (APC | | | |
| | (Allophycocyanin)) | | | |
| CD39 | PE Mouse anti-Human CD39 | 555464 | BD | TU66 |
| CD39 | CD39 Antibody, anti-human | 130-093-504 | Miltenyi | MZ18-23C8 |
| CD39 | CD39-PC5.5, BA54 | B55385 | Beckman Coulter | BA54 |

PD-1

| | | | | |
|---|---|---|---|---|
| PD-1 | Anti-CD279 (PD-1) Mouse | 329920 | BioLegend | EH12.2H7 |
| | Monoclonal Antibody | | | |
| | (Brilliant Violet ® 421) | | | |
| PD-1 | BB515 Mouse Anti-Human | 564494 | BD | EH12.1 |
| | CD279 (PD-1) | | | |
| PD-1 | CD279 (PD1) Antibody, anti- | 130-117-384 | Miltenyi | PD1.3.1.3 |
| | human | | | |
| PD-1 | CD279 (PD1) Antibody, anti- | 130-120-388 | Miltenyi | REA1165 |
| | human, REAfinity ™ | | | |
| PD-1 | CD279-PC5.5, PD1.3 | B32613 | Beckman Coulter | PD1.3 |

CD45

| | | | | |
|---|---|---|---|---|
| CD45 | APC-Cy7 Mouse Anti- | 348795 | BD | 2D1 |
| | Human CD45 | | | |
| CD45 | VioBright 515 Mouse Anti- | 130-110-640 | Miltenyi | REA747 |
| | Human CD45 | | | |

CD4

| | | | | |
|---|---|---|---|---|
| CD4 | Anti-CD4 Mouse | 317444 | BioLegend | OKT4 |
| | Monoclonal Antibody | | | |
| | (Brilliant Violet ® 510) | | | |
| CD4 | PE-CF594 Mouse Anti- | 566914 | BD | SK3 |
| | Human CD4 | | | |

CD8

| | | | | |
|---|---|---|---|---|
| CD8a | PE/Cy7 anti-human CD8a | 301012 | BioLegend | RPA-T8 |
| CD8 | PE Mouse Anti-Human CD8 | 340046 | BD | SK1 |

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. In some embodiments, a cell population described herein is collected and enriched (or depleted) via flow cytometry, in which cells stained for multiple cell surface markers (e.g., with an antibody-coupled fluorescent peptide) are carried in a fluidic stream.

In some embodiments, the cell staining involves incubation with an antibody or binding partner that specifically binds to such markers as described, which in some embodiments is followed by washing steps and separation of cells having bound the antibody or binding partner, from those cells having not bound to the antibody or binding partner. In some aspects of such processes, a volume of cells is mixed with an amount of a desired staining reagent and incubated under conditions for staining of the cells. In some embodiments, the staining or labelling is carried out at a temperature between 0° C. and 25° C., such as at or about 4° C. In some embodiments, the staining or labelling is carried out for greater than 5 minutes, typically greater than 15 minutes. In some embodiments, the staining or labelling is carried out for between 15 minutes and 6 hours, such as between 30 minutes and 2 hours. In some embodiments, the staining or labelling is carried out for example, at or about 15 minutes, 30 minutes, 1 hour, 1.5 hours, 2 hours, 2.5 hours, 3 hours, or any value between any of the foregoing. In some embodiments, the labeling with the one or more staining reagents is carried out simultaneously. In some embodiments, one or more wash steps are carried out prior to introducing the sample into the flow cytometer for analysis.

In some embodiments, the cell sample is prepared by suspending single cells at a density of 1×10⁶ to 5×10⁷ cells/ml in order to allow the cells to pass through the flow cytometer for reading. In some embodiments, the density of cells for sorting is 5×10⁶ cells/mL to 50×10⁶ cells/mL, such as at or about 20×10⁶ cells/mL. In some embodiments, this concentration of cells is called the fluid sheath. In some embodiments, the fluid sheath influences the rate of flow sorting, which typically progresses at around 2,000-20,000 cells (events) per second. The cell sample's) fluid sheath is typically made of a phosphate buffered saline solution, but other solutions are available as will be known and understood by those skilled in the art.

In some embodiments, the flow cytometry sorting rate. In some embodiments, the flow cytometry sorting rate is from 2000 events/second to 10,000 event/second. In some embodiments, the flow cytometry sorting rate is about 2000 events/second, about 3000 events/second, about 4,000 events/second, about 5000 events/second, about 6000 events/second, about 7000 events/second, about 8000 events/second, about 9000 events/second, about 10000 events/second, about 15000 events/second, or about 20000 events per second, or any value between any of the foregoing. In some embodiments, the flow cytometry sorting rate is about 6,000 events/second.

In some embodiments, the sample is introduced into a flow cytometer. The cell sample is typically narrowed down to a single stream through a fluidics system with the application of hydro pressure. This stream is then passed through the one or more beams of light scattering or fluorescence emission. Lasers typically serves as the light source in flow cytometers. The laser produces a single wavelength of light that once contacted with the cell sample produces scattered light in the forward direction as a measure of cell size, scattered light in the side direction as a measure of cell complexity, and fluorescent light, also emitted in the side direction which is proportional to the relative amount of a particular cell marker. Fluorescent channels are usually indicated by the designations FL1, FL2, FL3, etc., depending on the number of channels in the instrument. Each fluorescent channel is set with barrier filters to detect a selected specific dye while filtering out all others. The channel in which a specific dye is predominantly detectable may be referred to as its primary fluorescent channel while other fluorescent channels may be designated as secondary channels. Scattered and fluorescent emitted light signals are converted to electronic pulses that are processed by the flow cytometry engine and displayed on a graphical user interface “GUI.”

Methods of analyzing flowcytometric or FACS data can involve a “gating” for data to separate specific groups of cells. Different cell types can be identified by the scatter parameters and the fluorescence emissions resulting from labeling various cell proteins with dye-labeled antibodies as described above. The identification of clusters and, thereby, populations can be carried out by “gating” of the cells. In some embodiments, gates corresponding to subsets of particles of interest, such as TIL expressing markers of neoantigen reactivity, are defined by users with the aid of software operationally associated with the flow system as described above.

In some embodiments, a gate may be a “threshold” gate, which is a gate for only one optical parameter that defines an open region within the multidimensional space. In some embodiments, “threshold” gating can be used for forward light scatter to remove high frequency low level signals caused by interference, such as debris in the sample. In some embodiments, “window” gating is employed, e.g., by defining upper and lower bounds for signal values. In some embodiments, gating is carried out on a 2D-plot of two parameters, such as side scatter (e.g., on vertical axis) and a fluorescence signal (e.g., on horizontal axis).

In some embodiments, flow cytometry for a cell surface marker includes gating for an “F-minus one” (FMO) control. FMO gating includes separate portions of the same sample stained with a panel of detectably labeled binding agents that contains all the agents but one. The distribution of the signal of the removed fluorophore can be used to define the positive threshold for the missing label as it is known that all cells are negative in the control. The position of all gates can be determined using FMO controls in which the antibody against the investigated marker is substituted with an appropriate isotype control. In an exemplary method, a gate can be drawn using cells stained with the FMO cocktail around cells positive for CD45, CD4 and/or CD8, PD1 and CD39. In some embodiments, sequential gates can be used to arrive at the selected subpopulation. For example, methods can be carried out in which cells are gated on CD45 positive cells, then CD4 and/or CD8 positive cells, then on cells positive for PD-1 and CD39. In some embodiments, a viability dye also can be added. An exemplary viability dye is 7-ADD. In some embodiments, a gate can be drawn around cells negative for 7-AAD (7-AAD_{neg}).

In some embodiments, the cells are sorted into a single population of cells and collected. The selected population of cells are used as input for expansion, such as described in Section II.C.

The selection need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker.

In some embodiments, the enriched population of cells are enriched cells from a starting sample as describe above, in which the percentage of cells of a particular phenotype, e.g. tumor-reactive CD3+ T cells or CD3+ T cells surface positive for one or more T cell marker, e.g. PD-1 and CD39, in the enriched population of cells in increased by at least 10%, 20%, 30%, 40%, 50%, 60%, 70%, 80%, 90%, 100%, 500%, 1000%, 5000% or more greater than the percentage of such cells in the starting sample. In some embodiments, the purity of tumor-reactive CD3+ T cells or CD3+ T cells surface positive for PD-1 and CD39) versus total cells in the population of enriched cells, is at least 90%, 91%, 92%, 93%, 94%, and is generally at least 95%, 96%, 97%, 98%, 99% or greater.

### C. Stimulation of T Cells for Expansion

The provided methods further include expanding the population of selected TILs by culture with one or more T-cell stimulating agent of lymphocytes under conditions to produce a population of expanded T cells. Hence, the provided methods involve the ex vivo expansion and production of a T cell therapeutic composition, particularly for use in connection with treating cancer.

In some embodiments, the incubation or culturing with one or more T cell stimulatory agent(s) results in expansion or outgrowth of selected T cells, or a desired subset or subtype thereof or for viable cells thereof, for use in subsequent steps of the provided methods. Non-limiting examples of T cell stimulatory agent(s) and conditions for incubation or culture are described herein.

Thus, among the provided methods are methods of culturing T cells for manufacture of tumor reactive T cells in which T cells are cultured or incubated in the presence of a T cell stimulatory agent under conditions to expand T cells.

In some embodiments, the T cell stimulatory agent(s) include a recombinant T cell stimulating cytokine, such as IL-2, IL-7, IL-15, IL-21, IL-25, IL-23, IL-27 and/or IL-35. In some embodiments, the T cell stimulating cytokine includes IL-2, alone or in combination with another cytokine from among IL-7, IL-15, IL-21, IL-25, IL-23, IL-27 and/or IL-35. In some embodiments, the T cell stimulatory agent(s) include a recombinant T cell stimulating cytokine, such as IL-2, IL-7, IL-15, IL-21, IL-25 and/or IL-23. In some embodiments, the T cell stimulating cytokine includes IL-2, alone or in combination with another cytokine from among IL-7, IL-15, IL-21, IL-25 and/or IL-23. In some embodiments, the T cell stimulating cytokine is one, two, three or more of IL-2, IL-7, IL-15 and IL-21. In some embodiments, the T cell stimulating cytokine includes IL-2, alone or in combination with another cytokine from among IL-7, IL-15, and/or IL-21. In some embodiments, the T cell stimulating cytokine includes IL-2, alone or in combination with another cytokine from IL-25, IL-23, IL-27 and/or IL-35. In some embodiments, the T cell stimulating cytokines are IL-7 and IL-15.

In some embodiments, the choice of cytokine or combination of cytokines is within the level of a skilled artisan, so long as the cytokines or cytokines provide activity to stimulate the T cells to expand. The activity to stimulate tumor reactive T cells can be direct or indirect. In some embodiments, the one or more cytokines directly stimulate tumor reactive T cells to expand or proliferate. In some embodiments, the one or more cytokines suppress T regulatory T cells (Treg), thereby indirectly stimulating or enhancing proliferation of desired tumor reactive T cells.

In some embodiments, the T-cell stimulating agents include anti-CD3 (e.g. anti-CD3 antibody, such as OKT3), anti-CD28 reagents (e.g. anti-CD28 antibody), such as an anti-CD3 antibody (e.g. OKT3) and an anti-CD28 antibody and/or one or more recombinant cytokine (e.g. IL-2, IL-7, IL-21 and/or IL-15).

In some embodiments, the incubation with a T cell stimulatory agent(s) for the expansion does not include incubation with an agent or agents that engage CD3 and a costimulatory molecule, such as CD28. In some embodiments, the incubation with a T cell stimulatory agent(s) for the expansion does not include incubation with an anti-CD3 antibody, such as OKT3. In some embodiments, the incubation with a T cell stimulatory agent(s) for the expansion does not include incubation with an anti-CD3 (e.g. OKT3)/anti-CD28 antibody, presented by APC's, immobilized on a solid surface (e.g. bead), or as a soluble antibody. In some embodiments, the incubation with a T cell stimulatory agent(s) for the expansion does not include incubation with soluble anti-CD3, such as OKT3. In some embodiments, the incubation with a T cell stimulatory agent(s) for the expansion does not include incubation with an anti-CD3/anti-CD28, including such reagents immobilized on beads, e.g. as provided by Dynabeads. In some embodiments, the incubation with a T cell stimulatory agent(s) for the expansion does not include incubation with APCs, such as irradiated APCs. In some embodiments, the incubation with a T cell stimulatory agent(s) for the expansion does not include incubation with non-dividing PBMCs, such as irradiated PBMCs.

In some of any of the provided embodiments, the T cell stimulatory agent(s) is selected from an agent that initiates TCR/CD3 intracellular signaling and an agent that initiates signaling via a costimulatory receptor. In some of any of the provided embodiments, the agent that initiates TCR/CD3 intracellular signaling is an anti-CD3 antibody, such as OKT3. In some of any of the provided embodiments, the agent that initiates signaling via a costimulatory receptor comprises peripheral blood mononuclear cells (PBMCs), optionally non-dividing or irradiated PBMCs. In some of any of the provided embodiments, the agent that initiates signaling via a costimulatory receptor is an anti-CD28 antibody. In some of any of the provided embodiments, the T cell stimulatory agent(s) is an anti-CD3 antibody and an anti-CD28 antibody that each are soluble. In particular embodiments, one or more recombinant cytokines also are present as additional T cell stimulatory agents during the incubation. In some embodiments, the incubation with a T cell stimulatory agent(s) include incubation with at least one T cell stimulating recombinant cytokine (e.g. recombinant IL-2, IL-7, IL-21, IL-15, IL-25, IL-23, IL-27, and/or IL-35) and a further T cell stimulatory agent(s) that engage CD3 and/or a costimulatory molecule (e.g. CD28) on T cells.

In embodiments of the provided methods, the stimulating conditions include one or more agent, e.g., ligand, which turns on or initiates TCR/CD3 intracellular signaling cascade in a T cell and/or a costimulatory signal in a T cell. Such agents can include antibodies, such as those specific for a TCR component, e.g., anti-CD3, and/or costimulatory receptor, e.g. anti-CD28 or anti-4-1BB. In some embodiments, such agents are added to the culture medium as soluble antibodies. In other embodiments, such agents are bound to solid support such as a bead. In some embodiments, the T cell stimulatory agent(s) includes anti-CD3/CD28 conjugated magnetic beads (e.g., DYNABEADS® M-450 CD3/CD28 T Cell Expander).

An anti-CD3 antibody can include any antibody directed against or that can specifically bind the CD3 receptor on the surface of T cells, typically human CD3 on human T cells. Anti-CD3 antibodies include OKT3, also known as muromonab. Anti-CD3 antibodies also include the UHCTI clone, also known as T3 and CD3E. Other anti-CD3 antibodies include, for example, otelixizumab, teplizumab, and visilizumab. The anti-CD3 antibody can be added as a soluble reagent or bound to a bead. In particular embodiments, the anti-CD3 antibody is soluble.

In particular embodiments, the T cell stimulatory agent(s) include an anti-CD3 antibody, which is added to the cell culture medium during the incubation. In some embodiments, the anti-CD3 antibody is added at a concentration ranging between at or about 0.1 ng/mL and 50 ng/mL, such between at or about 0.5 ng/mL and at or about 50 ng/mL, between at or about 0.5 ng/mL and at or about 30 ng/mL, between at or about 0.5 ng/mL and at or about 15 ng/mL, between at or about 0.5 ng/mL and at or about 5 ng/mL, between at or about 0.5 ng/mL and at or about 1 ng/mL, between at or about 1 ng/mL and at or about 50 ng/mL, between at or about 1 ng/mL and at or about 30 ng/mL, between at or about 1 ng/mL and at or about 15 ng/mL, between at or about 1 ng/mL and at or about 5 ng/mL, between at or about 5 ng/mL and at or about 50 ng/mL, between at or about 5 ng/mL and at or about 30 ng/mL, between at or about 5 ng/mL and at or about 15 ng/mL, between at or about 15 ng/mL and at or 50 ng/mL, between at or about 15 ng/mL and at or about 30 ng/mL or between at or about 30 ng/mL and at or about 50 ng/mL, each inclusive.

In particular embodiments, the anti-CD3 antibody is OKT3. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 g/mL of OKT3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT3 antibody.

In some embodiments, the T cell stimulatory agent(s) includes incubation with an anti-CD3 antibody and incubation with a further agent that specifically binds to CD28 or stimulates or induces a CD28-mediated signal in cells. In some embodiments, the CD28− mediated signal can be initiated or provided by anti-CD28 antibody or antigen-binding fragment thereof. In some embodiments, the CD28-mediated signal can be provided by antigen-presenting feeder cells (APCs), such as peripheral blood mononuclear cells (PBMC).

In some embodiments, the T cell stimulatory agent(s) can include adding to the population of T cells feeder cells, such as non-dividing peripheral blood mononuclear cells (PBMC). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells. In some embodiments, the resulting population of cells contains at least about 5, 10, 20, or 40 or more PBMC feeder cells for each T lymphocyte in the initial population to be expanded. In some embodiments, the ratio of T cells to PBMCs and/or antigen-presenting cells is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500.

In some embodiments, the stimulation does not include incubation with PBMCs or other feeder cells, such as non-divided or irradiated PBMCs or other non-dividing or irradiated feeder cells.

In some embodiments, the T cell stimulatory agent(s) can include adding to the population of cells an anti-CD28 antibody or antigen-binding fragment thereof. An anti-CD28 antibody can include any antibody directed against or that can specifically bind the CD28 receptor on the surface of T cells. Non-limiting examples of anti-CD28 antibodies include NA/LE (e.g. BD Pharmingen), IM1376 (e.g. Beckman Coulter), or 15E8 (e.g. Miltenyi Biotec). The anti-CD28 antibody can be added as a soluble reagent or bound to a bead. In particular embodiments, the anti-CD3 antibody is soluble. In some embodiments, the anti-CD28 antibody is added at a concentration ranging between at or about 1 ng/mL and 1000 ng/mL, between at or about 1 ng/mL and 500 ng/mL, between at or about 1 ng/mL and at or about 100 ng/mL, between at or about 1 ng/mL and at or about 10 ng/mL, between at or about 10 ng/mL and at or about 1000 ng/mL, between at or about 10 ng/mL and at or about 500 ng/mL, between at or about 10 ng/mL and at or about 100 ng/mL, between at or about 100 ng/mL and at or about 1000 ng/mL, between at or about 100 ng/mL and at or about 500 ng/mL or between at or about 500 ng/mL and at or about 1000 ng/mL.

In some embodiments, the T cell stimulatory agent(s) include one or more recombinant cytokine. In some embodiments, the cytokine is added or is exogenous to the culture media. Thus, in some embodiments, one or more further recombinant cytokine also is included during the culturing. In some embodiments, the recombinant cytokine can include one or more of IL-2, IL-7, IL-15, IL-21, IL-25, IL-23, IL-27 and/or IL-35. In some embodiments, the recombinant cytokine can include one or more of IL-2, IL-7, IL-15, IL-21, IL-25 and/or IL-23. In some embodiments, the culturing and incubation is carried out in the presence of recombinant IL-2, IL-15 and IL-7. In some embodiments, the culturing is carried out in the presence of IL-2. In some embodiments, the culturing is carried out in the presence of IL-15 and IL-17, which, in some aspects does not additionally include IL-2. In particular embodiments, the recombinant cytokine(s) is human.

The recombinant cytokine generally is a recombinant human protein. In particular embodiments, the recombinant cytokine is present in the cell culture medium during the incubation at a concentration of at least or at least about 0.5 IU/mL, at least or at least about 1.0 IU/mL, at least or at least about 5 IU/mL, at least at or about or at or about 10 IU/mL, at least at or about or at or about 100 IU/mL, at least at or about or at or about 1000 IU/mL, at least at or about or at or about 1500 IU/mL, at least at or about or at or about 2000 IU/mL, at least at or about or at or about 2500 IU/mL, at least at or about or at or about 3000 IU/mL, at least at or about or at or about 3500 IU/mL, at least at or about or at or about 4000 IU/mL, at least at or about or at or about 4500 IU/mL, at least at or about or at or about 5000 IU/mL, at least at or about or at or about 5500 IU/mL, at least at or about or at or about 6000 IU/mL, at least at or about or at or about 6500 IU/mL, at least at or about or at or about 7000 IU/mL, at least at or about or at or about 7500 IU/mL, or at least at or about or at or about 8000 IU/mL. In an embodiment, the cell culture medium comprises between at or about 10 IU/mL and at or about 100 IU/mL, at or about 100 IU/mL and at or about 1000 IU/mL, at or about 1000 and at or about 2000 IU/mL, between at or about 2000 and at or about 3000 IU/mL, between at or about 3000 and 4000 at or about IU/mL, between at or about 4000 and at or about 5000 IU/mL, between at or about 5000 and at or about 6000 IU/mL, between at or about 6000 and at or about 7000 IU/mL, between at or about 7000 and at or about 8000 IU/mL, each inclusive.

In some embodiments, recombinant IL-2 is present in the cell culture medium. In some aspects, IL-2 is the only recombinant cytokine added to the culture. In some aspects, recombinant IL-2 and one other recombinant modulatory cytokine from IL-7, IL-15, IL-21, IL-23, IL-25, IL-27 or IL-35 is added to the culture. IL-2 is a cytokine that supports T cell recovery and proliferation. IL-2 also supports the homeostasis of T cells, thereby supporting their phenotype, differentiation status, and immune memory. In some cases, induction of regulatory T cells in the tumor microenvironment may lead to low bioavailability of IL-2. Recombinant IL-2 has been regularly used in broad expansion of T cells in various contexts. Recombinant IL-2 is commercially available. In particular embodiments, recombinant IL-2 is GMP grade (e.g. MACS GMP Recombinant Human IL-2, Miltenyi Biotec).

Recombinant IL-2 can be included in cell culture media during various stages of the provided process. In some cases, recombinant IL-2 can be included in the T cell expansion, such as to promote TIL outgrowth.

In some embodiments, recombinant IL-2 is added to the culture medium at a concentration between at or about 10 IU/mL and at or about 1000 IU/mL, such as between at or about 10 IU/mL and at or about 600 IU/mL, between at or about 10 IU/mL and at or about 400 IU/mL, between at or about 10 IU/mL and at or about 200 IU/mL, between at or about 10 IU/mL and at or about 100 IU/mL, between at or about 10 IU/mL and at or about 50 IU/mL, between at or about 50 IU/mL and at or about 1000 IU/mL, between at or about 50 IU/mL and at or about 600 IU/mL, between at or about 50 IU/mL and at or about 400 IU/mL, between at or about 50 IU/mL and at or about 200 IU/mL, between at or about 50 IU/mL and at or about 100 IU/mL, between at or about 100 IU/mL and at or about 1000 IU/mL, between at or about 100 IU/mL and at or about 600 IU/mL, between at or about 100 IU/mL and at or about 400 IU/mL, between at or about 100 IU/mL and at or about 200 IU/mL, between at or about 200 IU/mL and at or about 1000 IU/mL, between at or about 200 IU/mL and at or about 600 IU/mL, between at or about 200 IU/mL and at or about 400 IU/mL, between at or about 400 IU/mL and at or about 1000 IU/mL, between at or about 400 IU/mL and at or about 600 IU/mL or between at or about 600 IU/mL and at or about 1000 IU/mL. In some embodiments, recombinant IL-2 is present in an amount that is between 50 and 400 IU/mL.

In some embodiments, the expansion is carried out in the presence of recombinant IL-2 added at a concentration of between 200 IU/mL and at or about 5000 IU/mL. In some embodiments, recombinant IL-2 Is added to the culture medium at a concentration of at or about 200 IU/mL, at or about 300 IU/mL, at or about 400 IU/mL, at or about 500 IU/mL, at or about 600 IU/mL, at or about 700 IU/mL, at or about 800 IU/mL, at or about 900 IU/mL, at or about 1000 IU/mL, or any concentration between any of the foregoing. In some embodiments, recombinant IL-2 Is added to the culture medium at a concentration of at or about 300 IU/mL. In some embodiments, recombinant IL-2 is added to the culture medium at a concentration of at or about 600 IU/mL. In some embodiments, recombinant IL-2 is added to the culture medium at a concentration of at or about 1000 IU/mL. In some embodiments, at least one other recombinant modulatory cytokine from IL-7, IL-15, IL-21, IL-23, IL-25, IL-27 or IL-35 is added to the culture medium.

In some embodiments, the incubation is carried out with a higher dose IL-2. In some aspects, IL-2 is the only recombinant cytokine added to the culture.

In some embodiments, the recombinant IL-2 is added to the culture medium at a concentration between at or about 1000 IU/mL at or about 8000 IU/mL, such as between at or about 1000 IU/mL and at or about 7000 IU/mL, between at or about 1000 IU/mL and at or about 6000 IU/mL, between at or about 1000 IU/mL and at or about 5000 IU/mL, between at or about 1000 IU/mL and at or about 4000 IU/mL, between at or about 1000 IU/mL and at or about 2000 IU/mL, 2000 IU/mL at or about 8000 IU/mL, between at or about 2000 IU/mL and at or about 7000 IU/mL, between at or about 2000 IU/mL and at or about 6000 IU/mL, between at or about 2000 IU/mL and at or about 5000 IU/mL, between at or about 2000 IU/mL and at or about 4000 IU/mL, 4000 IU/mL at or about 8000 IU/mL, between at or about 4000 IU/mL and at or about 7000 IU/mL, between at or about 4000 IU/mL and at or about 6000 IU/mL, between at or about 4000 IU/mL and at or about 5000 IU/mL, between at or about 5000 IU/mL at or about 8000 IU/mL, between at or about 5000 IU/mL and at or about 7000 IU/mL, between at or about 5000 IU/mL and at or about 6000 IU/mL, between at or about 6000 IU/mL at or about 8000 IU/mL, between at or about 6000 IU/mL and at or about 7000 IU/mL or between at or about 7000 IU/mL and at or about 8000 IU/mL. In some embodiments, recombinant IL-2 is present in an amount that is or is about 3000 IU/mL.

In some embodiments, recombinant IL-15 is present in the cell culture medium. IL-15 is a cytokine that is involved in memory T cell homeostasis and activation. In some cases, IL-15 can promote effector functions of antigen-experienced T cells in the absence of antigen and prevent their differentiation into an exhausted phenotype. IL-15 also plays a role in T cell proliferation. Recombinant IL-15 is commercially available. In particular embodiments, recombinant IL-15 is GMP grade (e.g. MACS GMP Recombinant Human IL-15, Miltenyi Biotec).

Recombinant IL-15 can be included in cell culture media during various stages of the provided process. In some cases, recombinant IL-15 can also be included in cultures to expand tumor-reactive T cells during the expansion phase. In some cases, recombinant IL-15 can be combined with recombinant IL-7 to provide for activation, survival and/or expansion of tumor-reactive T cells in the provided methods. In some such embodiments, the combination of recombinant IL-7 and IL-15 is an alternative to the use of recombinant IL-2 in the culture, and the culture media does not additionally contain recombinant IL-2.

In some embodiments, the recombinant IL-15 is added to the culture medium at a concentration between at or about 10 IU/mL and 500 IU/mL, such as between at or about 10 IU/mL and at or about 400 IU/mL, between at or about 10 IU/mL and at or about 300 IU/mL, between at or about 10 IU/mL and at or about 200 IU/mL, between at or about 10 IU/mL and at or about 100 IU/mL, between at or about 10 IU/mL and at or about 70 IU/mL, between at or about 10 IU/mL and at or about 50 IU/mL, between at or about 10 IU/mL and at or about 30 IU/mL, between at or about 30 IU/mL and 500 IU/mL, between at or about 30 IU/mL and at or about 400 IU/mL, between at or about 30 IU/mL and at or about 300 IU/mL, between at or about 30 IU/mL and at or about 200 IU/mL, between at or about 30 IU/mL and at or about 100 IU/mL, between at or about 30 IU/mL and at or about 70 IU/mL, between at or about 30 IU/mL and at or about 50 IU/mL, between at or about 50 IU/mL and at or about 400 IU/mL, between at or about 50 IU/mL and at or about 500 IU/mL, between at or about 50 IU/mL and at or about 300 IU/mL, between at or about 50 IU/mL and at or about 200 IU/mL, between at or about 50 IU/mL and at or about 100 IU/mL, between at or about 50 IU/mL and at or about 70 IU/mL, between at or about 70 IU/mL and at or about 500 IU/mL, between at or about 70 IU/mL and at or about 400 IU/mL, between at or about 70 IU/mL and at or about 300 IU/mL, between at or about 70 IU/mL and at or about 200 IU/mL, between at or about 70 IU/mL and at or about 100 IU/mL, between at or about 100 IU/mL and at or about 500 IU/mL, between at or about 100 IU/mL and at or about 400 IU/mL, between at or about 100 IU/mL and at or about 300 IU/mL, between at or about 100 IU/mL and at or about 200 IU/mL, between at or about 200 IU/mL and at or about 500 IU/mL, between at or about 200 IU/mL and at or about 400 IU/mL, between at or about 200 IU/mL and at or about 300 IU/mL, between at or about 300 IU/mL and at or about 500 IU/mL, between at or about 200 IU/mL and at or about 400 IU/mL, or between at or about 400 IU/mL and at or about 500 IU/mL. In some embodiments, the IL-15 is added to the culture medium in an amount between at or about 100 IU/mL and at or about 200 IU/mL. In some embodiments, the IL-15 is added to the culture medium at or about 180 IU/mL.

In some embodiments, the incubation is carried out with a higher dose IL-15.

In some embodiments, the recombinant IL-15 is added to the culture medium at a concentration between at or about 500 IU/mL and at or about 5000 IU/mL, such as between at or about 500 IU/mL and at or about 4000 IU/mL, between at or about 500 IU/mL and at or about 2000 IU/mL, between at or about 500 IU/mL and at or about 1500 IU/mL, between at or about 500 IU/mL and at or about 1000 IU/mL, between at or about 500 IU/mL and at or about 750 IU/mL, between at or about 750 IU/mL and at or about 5000 IU/mL, between at or about 750 IU/mL and at or about 4000 IU/mL, between at or about 750 IU/mL and at or about 2000 IU/mL, between at or about 750 IU/mL and at or about 1500 IU/mL, between at or about 750 IU/mL and at or about 1000 IU/mL, between at or about 1000 IU/mL and at or about 5000 IU/mL, between at or about 1000 IU/mL and at or about 4000 IU/mL, between at or about 1000 IU/mL and at or about 2000 IU/mL, between at or about 1000 IU/mL and at or about 1500 IU/mL, between at or about 1500 IU/mL and at or about 5000 IU/mL, between at or about 1500 IU/mL and at or about 4000 IU/mL, between at or about 1500 IU/mL and at or about 2000 IU/mL, between at or about 2000 IU/mL and at or about 5000 IU/mL, such as between at or about 2000 IU/mL and at or about 4000 IU/mL, or between at or about 4000 IU/mL and at or about 5000 IU/mL. In some embodiments, the recombinant IL-15 is added to the cell culture media at a concentration of at or about 500 IU/mL, at or about 600 IU/mL, at or about 700 IU/mL, at or about 800 IU/mL, at or about 900 IU/mL, at or about 1000 IU/mL, at or about 1100 IU/mL, at or about 1200 IU/mL, at or about 1300 IU/mL, at or about 1400 IU/mL, at or about 1500 IU/mL, at or about 1600 IU/mL, at or about 1700 IU/mL, at or about 1800 IU/mL, at or about 1900 IU/mL or at or about 2000 IU/mL, or any concentration between any of the foregoing. In some embodiments, IL-15 is added to the culture medium at a concentration of at or about 1000 IU/mL.

In some embodiments, the expansion is carried out in the presence of recombinant IL-15 added at a concentration of 500 IU/mL to 2000 IU/mL (e.g. at or about 1000 IU/mL). In some embodiments, the expansion (is carried out in the presence of recombinant IL-15 added at a concentration of at or about 1000 IU/mL. In some embodiments, at least one other recombinant modulatory cytokine from IL-2, IL-7, IL-21, IL-23, IL-25, IL-27 or IL-35 is added to the culture medium.

In some embodiments, recombinant IL-15 and IL-2 are added to the culture medium. In some embodiments, recombinant IL-15 is added at a concentration of 500 IU/mL to 2000 IU/mL (e.g. at or about 1000 IU/mL) and recombinant IL-2 is added at a concentration of 200 IU/mL to 5000 IU/mL (e.g. at or about 3000 IU/mL). In some embodiments, the expansion is carried out in the presence of recombinant IL-15 added at 1000 IU/mL and recombinant IL-2 added at 3000 IU/mL. In some embodiments, at least one other recombinant modulatory cytokine from IL-7, IL-21, IL-23, IL-25, IL-27 or IL-35 is added to the culture medium.

In some embodiments, recombinant IL-7 is added to the culture medium. In some aspects, recombinant IL-7 is added to the culture media with one or both of IL-2 or IL-15. In some aspects, recombinant IL-7 and recombinant IL-2 are added to the culture media. In some aspects, recombinant IL-7 and recombinant IL-15 are added to the culture media. In some aspects, recombinant IL-7 (e.g. in combination with one or both of IL-2 and IL-15) and one other recombinant modulatory cytokine from IL-23, IL-25, IL-27 or IL-35 is added to the culture medium. IL-7 is a cytokine that is involved in promoting T cell maintenance and homeostasis. In some cases, IL-7 can boost memory T cell survival and proliferation, particularly the central memory compartment. Recombinant IL-7 is commercially available. In particular embodiments, recombinant IL-7 is GMP grade (e.g. MACS GMP Recombinant Human IL-7, Miltenyi Biotec).

Recombinant IL-7 can be included in cell culture media during various stages of the provided process. In some cases, recombinant IL-7 can also be included in cultures to expand tumor-reactive T cells during the expansion phase. Inclusion of recombinant IL-7 in the process can maintain or support expansion of memory T cell subsets in the process. In some cases, recombinant IL-7 can be combined with recombinant IL-15 to provide for activation, survival and/or expansion of tumor-reactive T cells in the provided methods. In some such embodiments, the combination of recombinant IL-7 and IL-15 is an alternative to the use of recombinant IL-2 in the culture, and the culture media does not additionally contain recombinant IL-2.

In some embodiments, the recombinant IL-7 is added to the culture medium at a concentration between at or about 100 IU/mL and at or about 2000 IU/mL, between at or about 100 IU/mL and at or about 1500 IU/mL, between at or about 100 IU/mL and at or about 1000 IU/mL, between at or about 100 IU/mL and at or about 800 IU/mL, between at or about 100 IU/mL and at or about 600 IU/mL, between at or about 100 IU/mL and at or about 400 IU/mL, between at or about 100 IU/mL and at or about 200 IU/mL, between at or about 200 IU/mL and at or about 2000 IU/mL, between at or about 200 IU/mL and at or about 1500 IU/mL, between at or about 200 IU/mL and at or about 1000 IU/mL, between at or about 200 IU/mL and at or about 800 IU/mL, between at or about 200 IU/mL and at or about 600 IU/mL, between at or about 200 IU/mL and at or about 400 IU/mL, between at or about 400 IU/mL and at or about 2000 IU/mL, between at or about 400 IU/mL and at or about 1500 IU/mL, between at or about 400 IU/mL and at or about 1000 IU/mL, between at or about 400 IU/mL and at or about 800 IU/mL, between at or about 400 IU/mL and at or about 600 IU/mL, between at or about 600 IU/mL and at or about 2000 IU/mL, between at or about 600 IU/mL and at or about 1500 IU/mL, between at or about 600 IU/mL and at or about 1000 IU/mL, between at or about 600 IU/mL and at or about 800 IU/mL, between at or about 800 IU/mL and at or about 2000 IU/mL, between at or about 800 IU/mL and at or about 1500 IU/mL, between at or about 800 IU/mL and at or about 1000 IU/mL, between at or about 1000 IU/mL and at or about 2000 IU/mL, between at or about 1000 IU/mL and at or about 1500 IU/mL, between at or about 1500 IU/mL and at or about 2000 IU/mL. In some embodiments, the IL-7 is added to the culture medium in an amount between at or about 1000 IU/mL and at or about 2000 IU/mL. In some embodiments, the IL-7 is added to the culture medium at or about 600 IU/mL. In some embodiments, IL-7 is added to the culture medium at or about 1000 IU/mL.

In some embodiments, recombinant IL-7 and IL-2 are added to the culture medium. In some embodiments, recombinant IL-7 is added at a concentration of 400 IU/mL to 2000 IU/mL (e.g. at or about 600 IU/mL or 1000 IU/mL) and recombinant IL-2 is added at a concentration of 200 IU/mL to 5000 IU/mL (e.g. at or about 3000 IU/mL). In some embodiments, the expansion is carried out in the presence of recombinant IL-7 added at 1000 IU/mL and recombinant IL-2 added at 3000 IU/mL. In some embodiments, the expansion is carried out in the presence of recombinant IL-7 added at 600 IU/mL and recombinant IL-2 added at 3000 IU/mL. In some embodiments, at least one other recombinant modulatory cytokine from IL-15, IL-21, IL-23, IL-25, IL-27 or IL-35 is added to the culture medium.

In some embodiments, recombinant IL-15 and IL-7 are added to the culture medium. In some embodiments, recombinant IL-15 is added at a concentration of 500 IU/mL to 2000 IU/mL (e.g. at or about 1000 IU/mL) and recombinant IL-7 is added at a concentration of 400 IU/mL to 2000 IU/mL (e.g. at or about 600 IU/mL or 1000 IU/mL). In some embodiments, the expansion is carried out in the presence of recombinant IL-15 added at 1000 IU/mL and recombinant IL-7 added at 1000 IU/mL. In some embodiments, the expansion is carried out in the presence of recombinant IL-15 added at 1000 IU/mL and recombinant IL-7 added at 600 IU/mL. In some embodiments, at least one other recombinant modulatory cytokine from IL-2, IL-21, IL-23, IL-25, IL-27 or IL-35 is added to the culture medium.

In some embodiments, recombinant IL-21 is added to the culture medium. In some aspects, recombinant IL-21 is added to the culture media with one or both of IL-2, IL-7, or IL-15. In some aspects, recombinant IL-21 and recombinant IL-2 are added to the culture media. In some aspects, recombinant IL-21 and recombinant IL-15 are added to the culture media. In some aspects, recombinant IL-21 (e.g. in combination with one or more IL-2, IL-7 and IL-15) and one other recombinant modulatory cytokine from IL-23, IL-25, IL-27 or IL-35 is added to the culture medium. IL-21 is a cytokine that supports a broad range of T cell activation without increasing regulatory T cell signaling. In some cases, IL-21 can support memory cell stabilization, effector function, and proliferation of antigen-experienced T cells. IL-21 can induce upregulation of effector molecules in both CD4 and CD8 T cells. Recombinant IL-21 is commercially available. In particular embodiments, recombinant IL-21 is GMP grade (e.g. MACS GMP Recombinant Human IL-21, Miltenyi Biotec).

Recombinant IL-21 can be included in cell culture media during various stages of the provided process. In some cases, recombinant IL-21 can also be included in cultures to expand tumor-reactive T cells during the expansion phase, such as to support proliferation and stabilization of memory phenotype.

In some embodiments, the recombinant IL-21 is added to the culture medium at a concentration between at or about 0.5 IU/mL and at or about 20 IU/mL, between at or about 0.5 IU/mL and at or about 15 IU/mL, between at or about 0.5 IU/mL and at or about 10 IU/mL, between at or about 0.5 IU/mL and at or about 5 IU/mL, between at or about 0.5 IU/mL and at or about 2.5 IU/mL, between at or about 0.5 IU/mL and at or about 1 IU/mL, between at or about 1 IU/mL and at or about 20 IU/mL, between at or about 1 IU/mL and at or about 15 IU/mL, between at or about 1 IU/mL and at or about 10 IU/mL, between at or about 1 IU/mL and at or about 5 IU/mL, between at or about 1 IU/mL and at or about 2.5 IU/mL, between at or about 2.5 IU/mL and at or about 20 IU/mL, between at or about 2.5 IU/mL and at or about 15 IU/mL, between at or about 2.5 IU/mL and at or about 10 IU/mL, between at or about 2.5 IU/mL and at or about 5 IU/mL, between at or about 5 IU/mL and at or about 20 IU/mL, between at or about 5 IU/mL and at or about 15 IU/mL, between at or about 5 IU/mL and at or about 10 IU/mL, between at or about 10 IU/mL and at or about 20 IU/mL, between at or about 10 IU/mL and at or about 15 IU/mL, or between at or about 15 IU/mL and at or about 20 IU/mL. In some embodiments, the IL-21 is added to the culture medium in an amount between at or about 0.5 IU/mL and at or about 2.5 IU/mL. In some embodiments, the IL-21 is added to the culture medium at or about 1 IU/mL.

In some embodiments, the incubation is carried out with a higher dose IL-21.

In some embodiments, the recombinant IL-21 is added to the culture medium at a concentration between at or about 500 IU/mL and at or about 5000 IU/mL, such as between at or about 500 IU/mL and at or about 4000 IU/mL, between at or about 500 IU/mL and at or about 2000 IU/mL, between at or about 500 IU/mL and at or about 1500 IU/mL, between at or about 500 IU/mL and at or about 1000 IU/mL, between at or about 500 IU/mL and at or about 750 IU/mL, between at or about 750 IU/mL and at or about 5000 IU/mL, between at or about 750 IU/mL and at or about 4000 IU/mL, between at or about 750 IU/mL and at or about 2000 IU/mL, between at or about 750 IU/mL and at or about 1500 IU/mL, between at or about 750 IU/mL and at or about 1000 IU/mL, between at or about 1000 IU/mL and at or about 5000 IU/mL, between at or about 1000 IU/mL and at or about 4000 IU/mL, between at or about 1000 IU/mL and at or about 2000 IU/mL, between at or about 1000 IU/mL and at or about 1500 IU/mL, between at or about 1500 IU/mL and at or about 5000 IU/mL, between at or about 1500 IU/mL and at or about 4000 IU/mL, between at or about 1500 IU/mL and at or about 2000 IU/mL, between at or about 2000 IU/mL and at or about 5000 IU/mL, such as between at or about 2000 IU/mL and at or about 4000 IU/mL, or between at or about 4000 IU/mL and at or about 5000 IU/mL. In some embodiments, the recombinant IL-21 is added to the cell culture media at a concentration of at or about 500 IU/mL, at or about 600 IU/mL, at or about 700 IU/mL, at or about 800 IU/mL, at or about 900 IU/mL, at or about 1000 IU/mL, at or about 1100 IU/mL, at or about 1200 IU/mL, at or about 1300 IU/mL, at or about 1400 IU/mL, at or about 1500 IU/mL, at or about 1600 IU/mL, at or about 1700 IU/mL, at or about 1800 IU/mL, at or about 1900 IU/mL or at or about 2000 IU/mL, or any concentration between any of the foregoing. In some embodiments, IL-21 is added to the culture medium at a concentration of at or about 1000 IU/mL.

In some embodiments, recombinant IL-21 and IL-2 are added to the culture medium. In some embodiments, recombinant IL-21 is added at a concentration of 500 IU/mL to 2000 IU/mL (e.g. at or about 1000 IU/mL) and recombinant IL-2 is added at a concentration of 200 IU/mL to 5000 IU/mL (e.g. at or about 3000 IU/mL). In some embodiments, the expansion is carried out in the presence of recombinant IL-21 added at 1000 IU/mL and recombinant IL-2 added at 3000 IU/mL. In some embodiments, at least one other recombinant modulatory cytokine from IL-7, IL-15, IL-23, IL-25, IL-27 or IL-35 is added to the culture medium.

In one aspect of provided methods, the tumor reactive T cells are directly sorted after tumor digest, and only a single expansion step is carried out, in which the population of expanded T cells is harvested as a therapeutic TIL composition. In such an example, tumor fragments are digested into a single cell suspension and provided as an input sample for sorting/selection for the tumor-reactive T cells thereof. Then, the selected cells are expanded and harvested as a therapeutic TIL composition. In some embodiments, the expansion is carried out for a period of time to achieve a therapeutic dose. In some embodiments, the expansion is carried to achieve a fold expansion of the cells of from at or about 200-fold to at or about 3000-fold. In some embodiments, the expansion is carried out to achieve a therapeutic dose of at or about or greater than at or about 500 million total cells. In some embodiments, the expansion is carried out for 1-28 days, such as for at or about 7 to 28 days, 7 to 21 days, 7 to 14 day, such as at or about 7 days, 8 days, 9 days, 10 days, 11 days, 13 days, 14 days, 15 days, 16 days, 17 days, 18 days, 19 days, 20 days, 21 days, 22 days, 23 days, 24 days, 25 days, 26 days, 27 days or 28 days.

In some embodiments, provided herein is a method for manufacturing tumor-reactive T cells comprising (a) selecting cells surface positive for PD-1 and/or CD39 from an input sample comprising T cells from a subject that has a tumor to obtain selected cells from the sample; and (b) performing an expansion by culture of the selected cells with one or more T-cell stimulating agent of lymphocytes under conditions to produce a population of expanded T cells. In some embodiments, the method includes harvesting the population of expanded T cells produced by the method for formulation as the therapeutic composition.

In some embodiments, provided herein is a method for manufacturing tumor-reactive T cells comprising (a) selecting cells surface positive for PD-1 and CD39 from an input sample comprising T cells from a subject that has a tumor to obtain selected cells from the sample; and (b) performing an expansion by culture of the selected cells with one or more T-cell stimulating agent of lymphocytes under conditions to produce a population of expanded T cells. In some embodiments, the method includes harvesting the population of expanded T cells produced by the method for formulation as the therapeutic composition.

In particular embodiments, T cell stimulatory agent(s) present during the incubation, such as for expansion of cells, contains recombinant IL-2. In some embodiments, one or more other stimulating agent can be included such as one or more other recombinant cytokine from IL-7, IL-15, IL-21, IL-25, and/or IL-23, or an anti-CD3 antibody (e.g. OKT-3). In some cases in which an anti-CD3 antibody (e.g. OKT-3) the T cell stimulating agent(s) also can include a costimulating agent, such as provided by antigen-presenting feeder cells, such as PBMCs, or a soluble anti-CD28 antibody.

In particular embodiments, T cell stimulatory agent(s) present during the incubation, such as for expansion of cells contains recombinant IL-2 and an anti-CD3 antibody.

In particular embodiments, T cell stimulatory agent(s) present during the incubation, such as for expansion of cells contains recombinant IL-2, an anti-CD3 antibody, e.g. OKT-3, and antigen-presenting feeder cells, such as PBMCs.

In particular embodiments, T cell stimulatory agent(s) present during the incubation, such as for expansion of cells contains recombinant IL-2, an anti-CD3 antibody, e.g. OKT-3, and an anti-CD28 antibody. In some embodiments, the anti-CD3 antibody and/or anti-CD28 antibody are soluble. In some embodiments, one or both of the anti-CD3 antibody and anti-CD28 antibody are bound to a solid surface, such as a bead (e.g., DYNABEADS® M-450 CD3/CD28 T Cell Expander).

In particular embodiments, T cell stimulatory agent(s) present during the incubation, such as for expansion of cells contains recombinant IL-2 an anti-CD3 antibody, e.g. OKT-3, and antigen-presenting feeder cells, such as PBMCs.

In particular embodiments, the incubation or culture of T cells also is carried out with nutrient containing media so that the cells can survive outside of the body. In embodiments of the provided methods, one or more of the steps can be carried out in serum-containing media, such as media containing human AB serum. The culture media containing the T cell stimulatory agent(s) can be a serum-free media.

In one embodiment, the serum free medium is OpTmizer CTS (LifeTech), Immunocult XF (Stemcell technologies), CellGro (CellGenix), TexMacs (Miltenyi), Stemline (Sigma), Xvivol5 (Lonza), PrimeXV (Irvine Scientific), or Stem XVivo (RandD systems). The serum-free medium can be supplemented with a serum substitute such as ICSR (immune cell serum replacement) from LifeTech. The level of serum substitute (e.g., ICSR) can be, e.g., up to 5%, e.g., about 1%, 2%, 3%, 4%, or 5%. In some embodiments, the serum-free media contains 0.5 mM to 5 mM of a dipeptide form of L-glutamine, such L-alanyl-L-glutamine (Glutamax™). In some embodiments, the concentration of the dipeptide form of L-glutamine, such as L-alanyl-L-glutamine, is from or from about 0.5 mM to 5 mM, 0.5 mM to 4 mM, 0.5 mM to 3 mM, 0.5 mM to 2 mM, 0.5 mM to 1 mM, 1 mM to 5 mM, 1 mM to 4 mM, 1 mM to 3 mM, 1 mM to 2 mM, 2 mM to 5 mM, 2 mM to 4 mM, 2 mM to 3 mM, 3 mM to 5 mM, 3 mM to 4 mM or 4 mM to 5 mM, each inclusive. In some embodiments, the concentration of the dipeptide form of L-glutamine, such as L-alanyl-L-glutamine, is or is about 2 mM.

In some embodiments, the cells are cultured at about 37° C. with about 5% C02.

In some embodiments, the incubation with the T cell stimulatory agent(s) is carried out for at or about 1 day, such as generally at or about 2 days, 3 days, 4 days, 5 days, 6 days, 7 days, 8 days, 9 days, 10 days, 11 days, 12 days, or any range of time between any of the foregoing. In some embodiments, the incubation with the T cell stimulatory agent(s) is carried out for 7 to 21 days, such as 7 days, 8 days, 9 days, 10 days, 11 days, 12 days, 13 days, 14 days, 15 days, 16 days, 17 days, 18 days, 19 days, 20 days or 21 days, or any value between any of the foregoing. In some embodiments, the incubation is carried out for 7-14 days. In some embodiments, the incubation is carried out for 7-10 days. In some embodiments, the incubation is for at or about 7 days. In some embodiments, the incubation is for at or about 8 days. In some embodiments, the incubation is for at or about 9 days. In some embodiments, the incubation is for at or about 10 days.

In some embodiments, the incubation with the T cell stimulatory agent(s) is a minimal expansion such that it does not result in downregulation of the T cell activation marker (e.g. PD-1 and/or CD39). For instance, the incubation with the T cell stimulatory agent(s) in the expansion is a short culture so that the markers CD39 and/or PD1 are still present during the sorting step and the cells have not downregulated those markers.

In some embodiments, the incubation with the T cell stimulatory agent(s), such as for the expansion of T cells in the input sample, is carried out for at or about 1 days. In some embodiments, the incubation with the T cell stimulatory agent(s), such as for the expansion of T cells in the input sample, is carried out for at or about 2 days. In some embodiments, the incubation with the T cell stimulatory agent(s), such as for the expansion of T cells in the input sample, is carried out for at or about 3 days. In some embodiments, the incubation with the T cell stimulatory agent(s), such as for the expansion of T cells in the input sample, is carried out for at or about 4 days. In some embodiments, the incubation with the T cell stimulatory agent(s), such as for the expansion of T cells in the input sample, is carried out for at or about 5 days. In some embodiments, the incubation with the T cell stimulatory agent(s), such as for the expansion of T cells in the input sample, is carried out for at or about 6 days. In some embodiments, the incubation with the T cell stimulatory agent(s), such as for the expansion of T cells in the input sample, is carried out for at or about 7 days.

The incubation, such as for expansion of T cells in the input sample, can be carried out under GMP conditions. In some embodiments, the incubation is in a closed system, which in some aspects may be a closed automated system. In some embodiments, the culture media containing the T cell stimulatory agent(s) can be a serum-free media. In some embodiments, the incubation is carried out in a closed automated system and with serum-free media.

In some embodiments, the expansion of cells under the one or more stimulatory conditions is in a culture vessel suitable for cell expansion. In some embodiments, the culture vessel is a gas permeable culture vessel, such as a G-Rex system (e.g. G-Rex 10, G-Rex 10M, G-Rex 100 M/100M-CS or G-Rex 500 M/500M-CS). In some embodiments the culture vessel is a microplate, flask, bar or other culture vessel suitable for expansion of cells in a closed system. In some embodiments, expansion can be carried out in a bioreactor. In some embodiments, the expansion can be carried out using a cell expansion system by transfer of the cells to gas permeable bags, such as in connection with a bioreactor (e.g. Xuri Cell Expansion System W25 (GE Healthcare)). In an embodiment, the cell expansion system includes a culture vessel, such as a bag, e.g. gas permeable cell bag, with a volume that is about 50 mL, about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L, or any value between any of the foregoing. In some embodiments, the process is automated or semi-automated. Examples of suitable bioreactors for the automated perfusion expansion include, but are not limited to, GE Xuri W25, GE Xuri W5, Sartorius BioSTAT RM 20|50, Finesse SmartRocker Bioreactor Systems, and Pall XRS Bioreactor Systems, or Miltenyi Prodigy. In some aspects, the expansion culture is carried out under static conditions. In some embodiments, the expansion culture is carried out under rocking conditions. The medium can be added in bolus or can be added on a perfusion schedule. In some embodiments, the bioreactor maintains the temperature at or near 37° C. and C02 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the culturing is performed with perfusion, such as with a rate of 290 ml/day, 580 ml/day, and/or 1160 ml/day.

In some embodiments, the cells are seeded in an appropriate culture vessel (e.g. gas permeable bag) at a density of from 0.5×10⁶ cells/mL to 1.5×10⁶ cells/mL. In some embodiments, the density is at or about 0.5×10⁶ cells/mL, 0.75×10⁶ cells/mL, 1×10⁶ cells/mL, 1.25×10⁶ cells/mL or 1.5×10⁶ cells/mL, or any value between any of the foregoing.

In some aspects, cells are expanded in an automated closed expansion system that is perfusion enabled. Perfusions can continuously add media to the cells to ensure an optimal growth rate is achieved.

The expansion methods can be carried out under GMP conditions, including in a closed automated system and using serum free medium. In some embodiments, any one or more of the steps of the method can be carried out in a closed system or under GMP conditions. In certain embodiments, all process operations are performed in a GMP suite. In some embodiments, a closed system is used for carrying out one or more of the other processing steps of a method for manufacturing, generating or producing a cell therapy. In some embodiments, one or more or all of the processing steps, e.g., isolation, selection and/or enrichment, processing, culturing steps including incubation in connection with expansion of the cells, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some embodiments, the stimulated cells are collected and are cryofrozen. In some embodiments, for cryopreservation, the stimulated cells are formulated as a composition with a cryoprotectant. In some embodiments, the cryoprotectant is or comprises DMSO and/or glycerol. In some embodiments, compositions formulated for cryopreservation can be stored at low temperatures, such as ultra low temperatures, for example, storage with temperature ranges from −40° C. to −150° C., such as or about 80° C.±6.0° C.

In some embodiments, the cryopreserved cells are prepared for subsequent steps by thawing.

### III. Methods of Treatment and Therapeutic Applications

Provided herein are compositions and methods relating to the provided therapeutic cell compositions described herein for use in treating diseases or conditions in a subject such as a cancer. Such methods and uses include therapeutic methods and uses, for example, involving administration of the therapeutic cells, or compositions containing the same, to a subject having a disease, condition, or disorder. In some cases, the disease or disorder is a tumor or cancer. In some embodiments, the cells or pharmaceutical composition thereof is administered in an effective amount to effect treatment of the disease or disorder. Uses include uses of the cells or pharmaceutical compositions thereof in such methods and treatments, and in the preparation of a medicament in order to carry out such therapeutic methods. In some embodiments, the methods thereby treat the disease or condition or disorder in the subject.

In some embodiments, the subject is the same subject from with the biological sample was obtained for producing the therapeutic cell composition. In some such embodiments, the provided methods of treatment is an adoptive cell therapy with a therapeutic composition containing T cells autologous to the subject.

In some embodiments, the cell compositions provided herein are autologous to the subject to be treated. In such embodiments, the starting cells for expansion are isolated directly from a biological sample from the subject as described herein, in some cases including with enrichment for T cells positive for one or more selection marker as described, and cultured under conditions for expansion as provided herein. In some aspects, the biological sample from the subject is or includes a tumor or lymph node sample and such sample tumor and an amount of such tissue is obtained, such as by resection or biopsy (e.g. core needle biopsy or fine-needle aspiration). In some embodiments, following the culturing under conditions for expansion in accord with the provided methods the cells are formulated and optionally cryopreserved for subsequent administration to the same subject for treating the cancer. In some embodiments, the methods of treatment comprise administering an effective amount of a composition containing tumor reactive CD3+ T cells or CD3+ T cells positive for one or more marker as described herein (e.g. PD-1/CD39). In some embodiments, the methods of treatment or uses of the compositions comprise administering a therapeutically effective amount of a composition containing tumor reactive CD3+ T cells or CD3+ T cells positive for one or more marker as described herein (e.g. PD-1/CD39) sufficient to treat, ameliorate or otherwise beneficially alter the symptoms of a condition, disorder or disease or other indication, such as, for example, any of the conditions, disorders or diseases described herein. Such compositions can include any as described herein, including compositions produced by the provided methods.

In some embodiments, a subject (e.g. autologous) is administered from at or about 10⁵ to at or about 10¹² CD3+ T cells produced by any of the provided methods, or from at or about 10⁵ to at or about 10⁸ CD3+ T cells produced by any of the provided methods, or from at or about 10⁶ and at or about 10¹² CD3+ T cells produced by any of the provided methods, or from at or about 10⁸ and at or about 10¹¹ CD3+ T cells produced by any of the provided methods, or from at or about 10⁹ and at or about 10¹⁰ CD3+ T cells produced by any of the provided methods. In some embodiments, the therapeutically effective amount for administration comprises greater than or greater than at or about 10⁵ CD3+ T cells produced by any of the provided methods, at or about 10⁶ CD3+ T cells produced by any of the provided methods, at or about 10⁷ CD3+ T cells produced by any of the provided methods, at or about 10⁸ CD3+ T cells produced by any of the provided methods, at or about 10⁹ CD3+ T cells produced by any of the provided methods, at or about 10¹⁰ CD3+ T cells produced by any of the provided methods, at or about 10¹¹ CD3+ T cells produced by any of the provided methods, or at or about 10¹² CD3+ T cells produced by any of the provided methods. In some embodiments, such an amount can be administered to a subject having a disease or condition, such as to a cancer patient. In some embodiments, the number of T cells are administered are viable T cells.

In some embodiments, the amount is administered as a flat dose. In other embodiments, the amount is administered per kilogram body weight of the subject. In some embodiments, the amount administered is a therapeutically effective amount sufficient to treat, ameliorate or otherwise beneficially alter the symptoms of a condition, disorder or disease or other indication. For example, the pharmaceutical T lymphocyte infiltrating (TIL) composition contains a therapeutically effective amount (e.g., flat dose or amount per kilogram body weight of a subject) of tumor-reactive T cells or T cells positive for a selection marker (e.g. PD-1/CD39) for treating a disease or condition such as a cancer, such as a tumor.

In some embodiments, the composition, such as produced by any of the provided methods or containing tumor-reactive T cells or T cells positive for a selection marker (e.g. PD-1/CD39), are administered to an individual soon after expansion according to the provided methods. In other embodiments, the expanded T cells, such as expanded tumor-reactive T cells or T cells positive for a selection marker, are cryopreserved prior to administration, such as by methods described above. For example, the T cells, such as tumor-reactive T cells or T cells positive for a selection marker, can be stored for greater than 6, 12, 18, or 24 months prior to administration to the individual. Such cryopreserved cells can be thawed prior to the administration.

In some embodiments, the provided compositions, such as provided by any of the provided methods or containing tumor-reactive T cells or T cells positive for a T cell selection marker, can be administered to a subject by any convenient route including parenteral routes such as subcutaneous, intramuscular, intravenous, and/or epidural routes of administration.

In some embodiments, the compositions, such as provided by any of the provided methods or containing tumor-reactive T cells or T cells positive for a selection marker may be administered in a single dose. Such administration may be by injection, e.g., intravenous injection. In some embodiments, tumor-reactive T cells or T cells positive for a selection marker may be administered in multiple doses. Dosing may be once, twice, three times, four times, five times, six times, or more than six times per year. Dosing may be once a month, once every two weeks, once a week, or once every other day. Administration of such compositions and cells may continue as long as necessary. In some embodiments, the compositions, such as provided by any of the provided methods or containing tumor-reactive T cells or T cells positive for a selection marker are administered in a therapeutically effective dose or amount sufficient to treat, ameliorate or otherwise beneficially alter the symptoms of a condition, disorder or disease or other indication.

In some embodiments, the subject is administered a lymphodepleting therapy prior to the administration of the dose of cells from a provided compositions, such as produced by any of the provided methods or containing tumor-reactive T cells or T cells positive for a selection marker. The lymphodepleting therapy can include administration of Fludarabine and/or cyclophosphamide (the active form being referred to as mafosfamide) and combinations thereof. Such methods are described in Gassner et al. (Cancer Immunol Immunother. 201 1, 60(1):75-85, Muranski, et al, Nat Clin Pract Oncol, 2006 3(12):668-681, Dudley, et al., J Clin Oncol 2008, 26:5233-5239, and Dudley, et al., J Clin Oncol. 2005, 23(10):2346-2357, all of which are incorporated by reference herein in their entireties. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day, or a dosage amount between a range of any of the foregoing. In some embodiments, the fludarabine is for 2-7 days, such as for 3-5 days, such as at or about 3 days, at or about 4 days or at or about 5 days. In some embodiments, the cyclophosphamide is administered at a dosage of 100 mg/m2/day, 150 mg/m2/day, 175 mg/m2/day, 200 mg/m2/day, 225 mg/m2/day, 250 mg/m2/day, 275 mg/m2/day, or 300 mg/m2/day. In some embodiments, the cyclophosphamide is administered intravenously (i.e., i.v.). In some embodiments, the cyclophosphamide treatment is for 2-7 days, such as 3-5 days, at or about 3 days, at or about 4 days or at or about 5 days. The lymphodepleting therapy is administered prior to the provided cell compositions. In some embodiments, the lymphodepleting therapy is carried out within a week of the administration of the provided cell compositions, such as 5-7 days prior to the administration of the dose of cells.

The compositions described herein can be used in a method for treating hyperproliferative disorders. In a preferred embodiment, they are for use in treating cancers. In some aspects, the cancer type may be including, but not limited to, ovarian, vulva, endometrial, urothelial, breast, pancreatic, prostate, colorectal, lung, brain, renal, stomach (gastrointestinal), and skin (including but not limited to squamous cell carcinoma, basal cell carcinoma, and melanoma). In some aspects, the cancer type may be including, but not limited to, ovarian, vulva, endometrial, urothelial, breast, colorectal, lung, renal, and skin (including but not limited to melanoma). In some aspects, the cancer type may be including, but not limited to, ovarian. In some aspects, the cancer type may be including, but not limited to, vulva. In some aspects, the cancer type may be including, but not limited to, endometrial. In some aspects, the cancer type may be including, but not limited to, urothelial. In some aspects, the cancer type may be including, but not limited to, breast. In some aspects, the cancer type may be including, but not limited to, colorectal. In some aspects, the cancer type may be including, but not limited to, lung. In some aspects, the cancer type may be including, but not limited to, renal. In some aspects, the cancer type may be including, but not limited to, skin (including but not limited to melanoma). In some embodiments, the cancer is an epithelial cancer. In some embodiments, the cancer is selected from non-small cell lung cancer (NSCLC), CRC, ovarian cancer, breast cancer, esophageal cancer, gastric cancer, pancreatic cancer, cholangiocarcinoma cancer, endometrial cancer. In some embodiments, the breast cancer is HR+/Her2− breast cancer. In some embodiments, the breast cancer is a triple negative breast cancer (TNBC). In some embodiments, the breast cancer is a HER2+ breast cancer.

In some embodiments, the subject has a cancer that is a hematological tumor. Non-limiting examples of hematological tumors include leukemia, including acute leukemias (such as 1 lq23− positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the subject has a solid tumor cancer. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the cancer is a skin cancer. In particular embodiments, the cancer is a melanoma, such as a cutaneous melanoma. In some embodiments, the cancer is a merkel cell or metastatic cutaneous squamous cell carcinoma (CSCC).

In some embodiments, the tumor is a carcinoma, which is a cancer that develops from epithelial cells or is a cancer of epithelial origin. In some embodiments, the cancer arises from epithelial cells which include, but are not limited to, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

In some embodiments, the subject has a cancer that is a gastrointestinal cancer involving a cancer of the gastrointestinal tract (GI tract), including cancers or the upper or lower digestive tract, or an accessory organ of digestion, such as esophagus, stomach, biliary system, pancreas, small intestine, large intestine, rectum or anus. In some embodiments, the cancer is an esophageal cancer, stomach (gastric) cancer, pancreatic cancer, liver cancer (hepatocellular carcinoma), gallbladder cancer, cancer of the mucosa-associated lymphoid tissue (MALT lymphoma), cancer of the biliary tree, colorectal cancer (including colon cancer, rectum cancer or both), anal cancer, or a gastrointestinal carcinoid tumor. In particular embodiments, the cancer is a colorectal cancer.

In some embodiments, the cancer is a colorectal cancer. Colorectal cancer (CRC) is a common tumor of increasing incidence, which, in many cases, does not response to checkpoint inhibition or other immunotherapy. This is the case even though such cancers have properties that are associated with response, e.g. a reasonably high mutation rate and well established association of prognosis with level of T cell infiltration.

In some embodiments, the cancer is an ovarian cancer. In some embodiments, the cancer is a triple-negative breast cancer (TNBC).

In some embodiments, the cancer is lung cancer. In some embodiments, the cancer is a breast cancer. In some embodiments, the cancer is a colorectal cancer. In some embodiments, the cancer is pancreatic cancer. In some embodiments, the cancer is a merkel cell cancer. In some embodiments, the cancer is a metastatic cutaneous squamous cell carcinoma (CSCC). In some embodiments, the cancer is a melanoma.

In some embodiments, the subject is one whose cancer is refractory to, and or who has relapsed following treatment with, a checkpoint blockade, such as an anti-PD1 or anti-PD-L1 therapy.

In some embodiments, the cell compositions provided herein are allogenic to the subject to be treated. In some aspects, the subject from which the cells are derived or isolated is a healthy subject or is not known to have a disease or conditions, such as a cancer. In such embodiments, the starting cells for expansion are isolated directly from a biological sample from such a subject as described herein, in some cases including with enrichment for T cells positive for one or more selection marker as described, and cultured under conditions for expansion as provided herein. In some aspects, the biological sample from the subject is or includes a tumor or lymph node sample and such sample tumor and an amount of such tissue is obtained, such as by resection or biopsy (e.g. core needle biopsy or fine-needle aspiration). In some embodiments, following the culturing under conditions for expansion the cells are formulated and optionally cryopreserved for subsequent administration to a different subject for treating a cancer in such different subject.

In some embodiments, the provided methods can be carried out with one or more other immunotherapies. In some embodiments, the immunotherapy is an immune modulating agent that is an immune checkpoint inhibitor. In some embodiments, the immune checkpoint inhibitor specifically binds a molecule selected from among CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28, TIGIT and VISTA. In some embodiments, the immune checkpoint inhibitor is and antibody or antigen-binding fragment, a small molecule or a polypeptide. In some embodiments, the immune checkpoint inhibitor is selected from among nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MEDI4736, MOXR0916, AMP-224, and MSB001078C, or is an antigen-binding fragment thereof.

In some embodiments, the provided methods include combination therapy of a cell therapy as described and PD-1 or PD-L1 inhibitors. A PD-1 or PD-L1 inhibitor can include binding antibodies, antagonists, or inhibitors (i.e., blockers).

In an embodiment, the PD-I inhibitor is nivolumab (commercially available as OPDIVO from Bristol-Myers Squibb Co.), or biosimilars, antigen-binding fragments, conjugates, or variants thereof. Nivolumab is a fully human IgG4 antibody blocking the PD-I receptor. In an embodiment, the anti-PD-I antibody is an immunoglobulin G4 kappa, anti-(human CD274) antibody. Nivolumab is assigned Chemical Abstracts Service (CAS) registry number 946414-94-4 and is also known as 5C4, BMS-936558, l†tIDX-1106, and ONO-4538. The preparation and properties of nivolumab are described in U.S. Pat. No. 8,008,449 and International Patent Publication No. WO 2006/121168.

In another embodiment, the PD-1 inhibitor comprises pembrolizumab (commercially available as KEYTRUDA from Merck & Co., Inc., Kenilworth, NJ, USA), or antigen-binding fragments, conjugates, or variants thereof. Pembrolizumab is assigned CAS registry number 1374853-91-4 and is also known as lambrolizumab, MK-3475, and SCH-900475. The properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2.

In an embodiment, the PD-LI inhibitor is durvalumab, also known as MEDI4736 (which is commercially available from Medimmune, LLC, Gaithersburg, JVIaryland, a subsidiary of AstraZeneca plc.), or antigen-binding fragments, conjugates, or variants thereof. In an embodiment, the PD-LI inhibitor is an antibody disclosed in U.S. Pat. No. 8,779,108 or U.S. Patent Application Publication No. 2013/0034559.

In an embodiment, the PD-LI inhibitor is avelumab, also known as MSB0010718C (commercially available from Merck KGaA/EMD Serono), or antigen-binding fragments, conjugates, or variants thereof. The preparation and properties of avelumab are described in U.S. Patent Application Publication No. US 2014/0341917 A1.

In an embodiment, the PD-LI inhibitor is atezolizumab, also known as MPDL3280A or RG7446 (commercially available as TECENTRIQ from Genentech, Inc., a subsidiary of Roche Holding AG, Basel, Switzerland), or antigen-binding fragments, conjugates, or variants thereof. In an embodiment, the PD-LI inhibitor is an antibody disclosed in U.S. Pat. No. 8,217,149, the disclosure of which is specifically incorporated by reference herein. In an embodiment, the PD-LI inhibitor is an antibody disclosed in U.S. Patent Application Publication Nos. 2010/0203056 A1, 2013/0045200 A1, 2013/0045201 A1, 2013/0045202 A1, or 2014/0065135 A1. The preparation and properties of atezolizumab are described in U.S. Pat. No. 8,217,149.

### IV. Kits and Articles of Manufacture

Provided herein are articles of manufacture and kits comprising the provided compositions, such as compositions containing T cells produced by any of the provided methods or containing or enriched for tumor-reactive T cells or T cells positive for a selection marker as described (e.g. PD-1/CD39). In some embodiments, the compositions are produced by any of the provided methods.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromagens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, optionally, other components included in the kit, and methods for using such. In some embodiments, the instructions indicate methods for using the cell compositions for administration to a subject for treating a disease or condition, such as in accord with any of the provided embodiments. In some embodiments, the instructions are provided as a label or a package insert, which is on or associated with the container. In some embodiments, the instructions may indicate directions for reconstitution and/or use of the composition.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

The term “allogeneic” as used herein means a cell or tissue that is removed from one organism and then infused or adoptively transferred into a genetically dissimilar organism of the same species.

The term “autologous” as used herein means a cell or tissue that is removed from the same organism to which it is later infused or adoptively transferred.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. Among the provided antibodies are antibody fragments.

An “antibody fragment” or “antigen-binding fragment” refers to a molecule other than a conventional or intact antibody that comprises a portion of a conventional or intact antibody containing at least a variable region that binds an antigen. Examples of antibody fragments include but are not limited to Fv, single chain Fvs (sdFvs), Fab, Fab′, Fab′-SH, F(ab′)₂; diabodies; linear antibodies; a single-domain antibodies comprising only the V_{H} region (VHH).

As used herein, “bind,” “bound” or grammatical variations thereof refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

The term “concurrently” is used herein to refer to a procedure, such as an incubation, selection, enrichment or administration, involving two or more agents, where at least part of the particular procedure with one agent overlaps in time with at least a second agent.

The term “intermittently” is used herein to refer to a procedure, such as an incubation, selection, enrichment or administration, involving two or more agents, where the particular procedure involving each agent do not occur at regular intervals or are not continuous or stop and start repeatedly with periods in between.

The term “sequentially” is used herein to refer to a procedure, such as an incubation, selection, enrichment or administration, involving two or more agents, where the particular procedure involving each agent do not overlap in time.

As used herein, “isolated” or “purified with reference to a peptide, protein or polypeptide refers to a molecule which is substantially free of all other polypeptides, contaminants, starting reagents or other materials, or substantially free from chemical precursors or other chemicals when chemically synthesized. Preparations can be determined to be substantially free if they appear free of readily detectable impurities as determined by standard methods of analysis, such as high-performance liquid chromatography (HPLC), thin-layer chromatography (TLC) or capillary electrophoresis (CE), used by those of skill in the art to assess such purity, or sufficiently pure such that further purification would not detectably alter the physical and chemical properties of the substance.

As used herein, the term “recombinant” refers to a cell, microorganism, nucleic acid molecule, or vector that has been modified by introduction of an exogenous, such as heterologous, nucleic acid molecule, or refers to a cell or microorganism that has been altered such that expression of an endogenous nucleic acid molecule or gene is controlled, deregulated or constitutive, where such alterations or modifications may be introduced by genetic engineering. Genetic alterations may include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule. The term “recombinant” also can refer to a protein product expressed from such a nucleic acid molecule or vector or from such cell or microorganism to which is introduced or modified with an exogenous nucleic acid.

As used herein, a “composition” refers to any mixture of two or more products, substances, or compounds, including cells. It may be a solution, a suspension, liquid, powder, a paste, aqueous, non-aqueous or any combination thereof.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

The term “pharmaceutical composition” refers to a composition suitable for pharmaceutical use in a mammalian subject, often a human. A pharmaceutical composition typically comprises an effective amount of an active agent (e.g., cells, such as expanded in accord with the provided methods) and a carrier, excipient, or diluent. The carrier, excipient, or diluent is typically a pharmaceutically acceptable carrier, excipient or diluent, respectively.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Reference to “population of cells” herein is meant to refer to a number of cells that share a common trait. A population of cells generally contains a plurality of cells, such as greater than at or about 100 cells, at or about 1000 cells, and typically range from 1×10⁴ to 1×10¹⁰ in number.

The term “soluble” as used herein in reference to proteins, means that the protein is not bound, immobilized or attached to a particle, such as a cell or solid support, e.g. a bead. For example, a soluble protein includes a protein that is not bound as a transmembrane protein to the cell membrane of a cell. In some cases, solubility of a protein can be improved by linkage or attachment, directly or indirectly via a linker, to another molecule such as an Fc domain, which, in some cases, also can improve the stability and/or half-life of the protein. In some aspects, a soluble protein is an Fc fusion protein.

The term “specifically binds” as used herein means the ability of a protein, under specific binding conditions, to bind to a target protein such that its affinity or avidity is at least 10 times as great, but optionally 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to more than one target molecule. In some cases, a specifically binding protein may bind to a protein that has similarity in structural conformation with the target protein (e.g., paralogs or orthologs). Those of skill will recognize that specific binding to a molecule having the same function in a different species of animal (i.e., ortholog) or to a molecule having a substantially similar epitope as the target molecule (e.g., paralog) is possible and does not detract from the specificity of binding which is determined relative to a statistically valid collection of unique non-targets (e.g., random polypeptides). Solid-phase ELISA immunoassays, ForteBio Octet or Biacore measurements can be used to determine specific binding between two proteins. Generally, interactions between two binding proteins have dissociation constants (Kd) less than about 1×10⁻⁵ M, and often as low as about 1×10⁻¹² M. In certain aspects of the present disclosure, interactions between two binding proteins have dissociation constants of less than about 1×10⁻⁶ M, 1×10⁻⁷ M, 1×10⁻⁸ M, 1×10⁻⁹ M, 1×10⁻¹⁰ M, or 1×10⁻¹¹ M or less.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, a “subject” is a mammal, such as a human or other animal, and typically is human. The subject can be male or female and can be any suitable age, including infant, juvenile, adolescent, adult, and geriatric subjects.

The terms “effective amount” or “therapeutically effective amount” refer to a quantity and/or concentration of a therapeutic composition, such as containing cells, e.g. expanded in accord with the provide methods, that when administered to a patient yields any manner in which the symptoms of a condition, disorder or disease or other indication, are ameliorated or otherwise beneficially altered. An effective amount for treating a disease or disorder may be an amount that relieves, lessens, or alleviates at least one symptom or biological response or effect associated with the disease or disorder, prevents progression of the disease or disorder, or improves physical functioning of the patient. In particular aspects, there is a statistically significant inhibition of disease progression as, for example, by ameliorating or eliminating symptoms and/or the cause of the disease. In the case of cell therapy, the effective amount is an effective dose or number of cells administered to a patient. In some embodiments the patient is a human patient.

As used herein, “disease,” disorder” or “condition” refers to a pathological condition in an organism resulting from cause or condition including, but not limited to, infections, acquired conditions, genetic conditions, and characterized by identifiable symptoms. In particular, it is a condition where treatment is needed and/or desired.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of an immunomodulatory protein or engineered cells of the present invention either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. “Preventing,” “prophylaxis,” or “prevention” of a disease or disorder as used in the context of this invention refers to the administration of an immunomodulatory protein or engineered cells expressing an immunomodulatory protein of the present invention, either alone or in combination with another compound, to prevent the occurrence or onset of a disease or disorder or some or all of the symptoms of a disease or disorder or to lessen the likelihood of the onset of a disease or disorder. For example, in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS).

The term “antigen” refers to a molecule that can induce an immune response. Typically, an antigen is a molecule that is capable of being bound by a recognition site on an immune molecule, such as an antibody or T cell receptor if presented by major histocompatibility complex (MHC) molecules. An antigen can have one or more epitopes in which each epitope that is part of the antigen can be bound by a recognition site of an antibody or TCR/MHC complex. In some embodiments, an antigen is capable of inducing a humoral immune response or a cellular immune response leading to the activation of B lymphocytes and/or T lymphocytes.

As used herein, a “tumor-associated antigen” or “tumor-specific antigen” refers to a protein or other molecule that is found only on cancer cells and not on normal cells.

The term “in vivo” refers to an event that takes plane in a mammalian subject's body.

The term “ex vivo” refers to an event that takes place on or in a tissue or cells from a mammalian subject but outside of the mammalian subject's body. Typically, the event is carried out in an external environment. In particular aspects, an ex vivo procedure includes any in which an organ, cell or tissue is taken from a subject, typically a living body, for a treatment or procedure and then returned to the subject.

The term “in vitro” refers to an event that takes place in a test system, such as in a laboratory.

As used herein, a kit is a packaged combination that optionally includes other elements, such as additional reagents and instructions for use of the combination or elements thereof.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “article of manufacture” is a product that is made and, in some cases, that can be sold. In some embodiments, the term can refer to compositions contained in articles of packaging, such as in a container.

It is understood that aspects and embodiments of the invention described herein include “comprising,” “consisting,” and “consisting essentially of” aspects and embodiments.

### VI. Exemplary Embodiments

Among the provided embodiments are:
1. A pharmaceutical T lymphocyte infiltrating (TIL) composition enriched in tumor reactive T cells, the composition comprising a population of tumor infiltrating T cells comprising CD4+ and CD8+ T cells from a tumor, wherein at least 90% of cells in the composition are CD3+ T cells and less than about 5% of the population are T regulatory cells.
2. A pharmaceutical T lymphocyte infiltrating (TIL) composition enriched in tumor reactive T cells, the pharmaceutical composition comprising an oligoclonal population of tumor infiltrating T cells comprising CD4+ and CD8+ T cells from a tumor, wherein up to 40 clones make up at least 40% of the TCR frequency in the population.
3. The pharmaceutical composition of embodiment 2, wherein less than about 5% of the population are regulatory T cells.
4. The pharmaceutical composition of any of embodiments 1-3, wherein less than about 3% of the population are T regulatory cells.
5. The pharmaceutical composition of any of embodiments 1-4, wherein less than about 1% of the population are regulatory T cells.
6. The pharmaceutical composition of any of embodiments 1 and 3-5, wherein a T regulatory cell phenotype is characterized by surface marker expression of CD4+ CD8-CD25+ Foxp3+ CD127low.
7. The pharmaceutical composition of any of embodiments 1-6, wherein T cells of the population express PD-1 and/or CD39.
8. The pharmaceutical composition of embodiment 7, wherein the percentage of cells that express surface marker PD-1 in the composition, or of viable cells thereof, is about at least 30%, at least 40%, at least 50%, at least 60%, at least 70%, at least 80%, at least 90%, at least 100%, of the cells in the composition.
9. The pharmaceutical composition of embodiment 7 or embodiment 8, wherein the percentage of cells that express surface marker CD39 in the composition, or of viable cells thereof, is about at least 30%, at least 40%, at least 50%, at least 60%, at least 70%, at least 80%, at least 90%, at least 100%, of the cells in the composition.
10. The pharmaceutical composition of embodiment 7, wherein the percentage of cells that express surface marker PD-1 and CD39 in the composition, or of viable cells thereof, is about at least 30%, at least 40%, at least 50%, at least 60%, at least 70%, at least 80%, at least 90%, at least 100%, of the cells in the composition.
11. The pharmaceutical composition of any of embodiments 2-10, wherein at least 90% of the cells in the composition are CD3+ T cells.
12. The pharmaceutical composition of any of embodiments 1-11, wherein at least 95% of the cells in the population are CD3+ T cells.
13. The pharmaceutical composition of any of embodiments 1-11, wherein at least 98% of the cells in the population are CD3+ T cells.
14. The pharmaceutical composition of any of embodiments 1-13, wherein at least 80% of the cells in the composition are CD3+ CD56− T cells.
15. The pharmaceutical composition of any of embodiments 1-14, wherein greater than 60% of the cells of the population are T effector memory cells.
16. The pharmaceutical composition of any of embodiments 1-15, wherein greater than 75% of the cells of the population are T effector memory cells.
17. The pharmaceutical composition of any of embodiments 1-15, wherein greater than 80% of the cells of the population are T effector memory cells.
18. The pharmaceutical composition of any of embodiments 1-15, wherein greater than 85% of the cells of the population are T effector memory cells.
19. The pharmaceutical composition of any of embodiments 1-15, wherein greater than 90% of the cells of the population are T effector memory cells.
20. The pharmaceutical composition of any of embodiments 15-19, wherein the effector memory phenotype is characterized by surface marker expression of one or more of CD45RA⁻, CD45RO+, CD62L⁻, CCR7−, CD28− and CD27−.
21. The pharmaceutical composition of any of embodiments 15-20, wherein the effector memory phenotype is characterized by surface marker expression CD45RA⁻, CD45RO+, CD62L⁻, and CCR7−.
22. The pharmaceutical composition of any of embodiments 15-20, wherein the effector memory phenotype is characterized by surface marker expression CD45RA⁻, CD45RO⁺, CD62L, CCR7⁻, CD28⁻ and CD27⁻.
23. The pharmaceutical composition of any of embodiments 15-20, wherein the effector memory phenotype is characterized by surface marker expression CD45RA⁻ CCR7−⁻.
24. The pharmaceutical composition of any of embodiments 2-23, wherein up to 40 TCR clonotypes make up at least 50% of the TCR frequency in the population, optionally wherein the top 40 clonotypes make up at least 50% of the TCR frequency in the population.
25. The pharmaceutical composition of any of embodiments 2-23, wherein up to 40 TCR clonotypes make up at least 60% of the TCR frequency in the population, optionally wherein the top 40 clonotypes make up at least 60% of the TCR frequency in the population.
26. The pharmaceutical composition of any of embodiments 1-25, wherein the TCR clonotypes exhibit reactivity for at least one CD8 antigen and at least one CD4 antigen.
27. The pharmaceutical composition of any of embodiments 1-26, wherein at least 20% of the CD8+ T cells and/or at least 20% of the CD4+ T cells in the composition exhibit neoantigen reactivity.
28. The pharmaceutical composition of any of embodiments 1-27, wherein, the TIL composition is characterized by at least one of the following criteria in an in vitro autologous tumor assay:
i) IFN-γ production that is greater than 2000 pg/mL;
ii) granzyme B production in the supernatant that is greater than 200 pg/mL;
iii) greater than 10% tumor cell killing.
29. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least about 90% of cells in the composition are CD3+ T cells and wherein, the TIL composition is characterized by at least one of the following criteria in an in vitro autologous tumor assay:
   i) IFN-γ production that is greater than 2000 pg/mL;
   ii) granzyme B production that is greater than 500 pg/mL;
   iii) greater than 15% killing of the autologous tumor cells.
30. The pharmaceutical composition of embodiment 28 or embodiment 29, wherein the TIL composition is characterized by criteria (i) and (ii).
31. The pharmaceutical composition of embodiment 28 or embodiment 30, wherein the TIL composition is characterized by criteria (i) and (iii).
32. The pharmaceutical composition of embodiment 28 or embodiment 30, wherein the TIL composition is characterized by criteria (ii) and (iii).
33. The pharmaceutical composition of embodiment 28 or embodiment 30, wherein the TIL composition is characterized by criteria (i), (ii) and (iii).
34. The pharmaceutical composition of any of embodiments 28-33, wherein IFN-y production is greater than 3000 pg/mL or greater than 4000 pg/mL.
35. The pharmaceutical composition of any of embodiments 28-34, wherein granzyme B production is greater than 400 pg/mL or greater than 500 pg/mL.
36. The pharmaceutical composition of any of embodiments 28-35, wherein killing of the autologous tumor cells is greater than 40%.
37. The pharmaceutical composition of any of embodiments 1-36, wherein the composition is characterized by a greater number of CD4+ T cells than CD8+ T cells.
38. The pharmaceutical composition of any of embodiments 1-37, wherein a ratio of CD4+ T cells to CD8+ T cells in the composition is between 5:1 to 1:5.
39. The pharmaceutical composition of any of embodiments 1-38, wherein a ratio of CD4+ T cells to CD8+ T cells in the composition is between 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1.
40. The pharmaceutical composition of any of embodiments 1-39, wherein a ratio of CD4+ T cells to CD8+ T cells in the composition is at or about 10:1 to 25:1, optionally at or about 20:1.
41. The pharmaceutical composition of any of embodiments 1-40, wherein the number of cells in the composition is a therapeutically effective amount of TILs.
42. The pharmaceutical composition of any of embodiments 1-41, wherein the number of cells in the composition, or of viable cells thereof, is at least 2×10⁷ cells.
43. The pharmaceutical composition of any of embodiments 1-42, wherein the number of cells in the composition, or of viable cells thereof, is between at or about 2×10⁷ cells and 20×10⁹ cells, 2×10⁷ cells and 10×10⁹ cells, 2×10⁷ cells and 2×10⁹ cells, 2×10⁷ cells and 2×10⁸ cells, 2×10⁸ cells and 20×10⁹ cells, 2×10⁸ cells and 10×10⁹ cells, 2×10⁸ cells and 2×10⁹ cells, 2×10⁹ cells and 20×10⁹ cells, 2×10⁹ cells and 10×10⁹ cells, or 10×10⁹ cells and 20×10⁹ cells, each inclusive.
44. The pharmaceutical composition of any of embodiments 1-43, wherein the pharmaceutical composition is for treatment of a patient's tumor.
45. The pharmaceutical composition of any of embodiments 1-44, wherein the tumor is a colorectal cancer (CRC) tumor, a melanoma tumor, a non-small cell lung cancer (NSCLC) tumor, or an ovarian cancer tumor.
46. The pharmaceutical composition of any of embodiments 1-45, wherein the tumor is from a human subject.
47. The pharmaceutical composition of embodiment 46, wherein the pharmaceutical composition is for autologous adoptive therapy to the human subject.
48. The pharmaceutical composition of any of embodiments 1-47, comprising a pharmaceutically acceptable excipient.
49. The pharmaceutical composition of any of embodiments 1-48, comprising a cryoprotectant.
50. The pharmaceutical composition of any of embodiments 1-49, wherein the composition is a liquid composition.
51. The pharmaceutical composition of embodiment 50, wherein the composition had been frozen and thawed.
52. The pharmaceutical composition of any of embodiments 1-51, wherein the volume of the composition is between 1 mL and 500 mL.
53. The pharmaceutical composition of any of embodiments 1-52, wherein the composition is frozen.
54. The pharmaceutical composition of any of embodiments 1-53, wherein the composition is prepared by selecting cells surface positive for PD-1 and CD39 from cells obtained from a tumor of a donor subject and expanding the cells ex vivo.
55. The pharmaceutical composition of any of embodiments 1-53, wherein the composition is prepared by a method comprising:
a. providing dissociated tumor cells from a tumor obtained from a donor subject, wherein the dissociated tumor cells are a first population of cells that comprise CD4+ and CD8+ T cells;
b. selecting, from the first population of cells, cells that are surface positive for CD45, PD1 and CD39 and a T cell marker to produce a population of selected T lymphocyte infiltrating cells (TILs), optionally wherein the T cell marker is CD4 or CD8; and
c. expanding the population of selected TILs by culture with one or more T-cell stimulating agent of lymphocytes under conditions to produce a population of expanded T cells.
56. A method of producing a T lymphocyte infiltrating (TIL) composition enriched in tumor reactive T cells, the method comprising:
a. providing dissociated tumor cells from a tumor obtained from a donor subject, wherein the dissociated tumor cells are a first population of cells that comprise CD4+ and CD8+ T cells;
b. selecting, from the first population of cells, cells that are surface positive for CD45, PD1 and CD39 and a T cell marker to produce a population of selected T lymphocyte infiltrating cells (TILs), optionally wherein the T cell marker is CD4 or CD8; and
c. expanding the population of selected TILs by culture with one or more T-cell stimulating agent of lymphocytes under conditions to produce a population of expanded T cells.
57. The method of embodiment 55 or embodiment 56, wherein the one or more T-cell stimulating agent is selected from one or more of allogenic feeder cells, anti-CD3 antibody, and recombinant IL-2.
58. The method of any of embodiments 55-57, wherein the dissociated tumor cells are a single cell suspension processed by homogenization and enzymatic digestion of one or more tumor fragments from the resected tumor.
59. The method of embodiment 58, wherein the enzymatic digestion is by incubation with a collagenase, optionally at a concentration of about 10 mg/ml, and a hyaluronidase, optionally at a concentration of about 10 mg/ml.
60. The method of any of embodiments 55-59, wherein the concentration of the first population of cells is between 5×10⁶ cells/mL and 50×10⁶ cells/mL, optionally about 20×10⁶ cells/mL.
61. The method of any of embodiments 55-60, wherein selecting cells is performed using a microfluidics chip based cell sorting comprising at least 4 fluorescence detectors.
62. The method of embodiment 61, wherein selecting the cells comprises sorting the cells positive for the at least 4 fluorescence signal based on a fluorescence minus one (FMO) cocktail.
63. The method of embodiment 62, wherein the sorting is performed at a rate of 5,000 events per second to 10,000 events per second, optionally at about 6,000 events per second.
64. A method of treating a subject having a cancer, the method comprising administering to a subject having a tumor a therapeutically effective dose of the composition of any of embodiments 1-55.
65. The method of embodiment 64, wherein the therapeutically effective dose is between about 1×10⁹ and 10×10⁹ T cells.
66. The method of embodiment 65, wherein the therapeutically effective dose is from more than 1 million to less than 100 million T cells per kilogram of body weight.
67. The method of embodiment 65, wherein the therapeutically effective dose is from more than 1 million to less than 10 million T cells per kilogram of body weight.
68. The method of embodiment 65, wherein the therapeutically effective dose is from at or about 10 million to at or about 50 million T cells per kilogram of body weight.
69. The method of any of embodiments 64-68, wherein the cells of the therapeutic composition are autologous to the subject.

### VII. Examples

The following examples are included for illustrative purposes only and are not intended to limit the scope of the invention.

### Example 1: Recovery of PD1+CD39+ Tumor Infiltrating Lymphocytes (TILs) Via Cell Sorting from Tumor Samples

Fresh or frozen tumors from patients with colorectal cancer (CRC), ovarian, non-small cell lung cancer (NSCLC), kidney, breast, vulva, urothelial, endometrial or melanoma were processed as described below and resultant infiltrating T-cell populations were analyzed for cell count viability. Tumors were sourced from primary tumors in patients with cancer and shipped overnight in HypoThermosol at 4° C. Tumors were processed as single cell suspension (SCS) cultures.

For SCS generation, tumors were minced into fragments approximately 3 mm in diameter. Fragments were then homogenized in a closed system using the Miltenyi GentleMACS in the presence of an enzyme cocktail to digest the tumor, comprising of Collagenase (Sigma, #C5138, 10 mg/ml), DNase I (Sigma, #D5025, 10000 IU/ml), and Hyaluronidase (Sigma, #H2126, 10 mg/ml) in 4 mL of CTS Optimizer Media (Life Technologies, #A10221-01) supplemented with CTS Optimizer expansion supplement (Life Technologies, #A10484-02), CTS Immune Cell SR (Life Technologies, #A2596101), Gentamicin, and GlutaMax (ThermoFisher, #35050-061) (herein referred to as CTS media). Fragments designated for SCS with homogenization and enzyme digestion were incubated with the enzyme cocktail for a total of 60 minutes. The tumor contents were then processed into a single suspension through a 70 micron cell strainer. Immediately following the generation of SCS, cell counts and viability assessments were performed on the NC-200 Automated Cell Counter (ChemoMetec).

TILs from tumor samples were sorted by Fluorescence Activated Cell Sorting (FACS) as described below.

Single cell suspensions of the various tumor samples were prepared as described above. When frozen SCS were used, the SCS cells were thawed and washed in CTS media with benzonase to avoid cell clumping. Viable cells were then transferred and incubated with Fc Block at room temperature for 10 minutes. For the sorting process using the FX500 sorter, cells were washed and stained with either a cocktail of commercial antibodies, including anti-CD45 APC-Cy7 (BD), anti-CD4 PE-CF594 (BD), anti-CD8 PE (BD), anti-PD-1 BB515 (BD), anti-CD39 AF647 (BD), a fluorescence minus one (FMO) cocktail, or remained unstained. Cells were brought to a concentration of approximately 20×10⁶ cells/mL and sorted at a sort rate of approximately 6,000 events per second. For the sorting process using the MA900 a live/dead dye (7-AAD) was used. Ten minutes before sorting, 1 μl of 7-AAD stain was added per 100 μl of cell solution to measure cell viability. A gate was drawn using cells stained with the FMO cocktail around cells positive for CD45, CD4 and/or CD8, PD1 and CD39, as well as negative for 7-AAD (7-AAD_{neg}), and sorted into a single population (Table E1). This was the live, positive sorted population. Additionally, unselected cells were sorted into a separate population, and were identified as 7-AAD_{neg}CD45ₚₒₛPD1ₚₒₛCD39ₚₒₛ, 7-AAD_{neg}CD45ₚₒₛPD1ₚₒₛCD39_{neg} and/or 7-AAD_{neg}CD45ₚₒₛPD1_{neg}CD39_{neg}. Cells lacking both 7-AAD and CD45 expression were sorted into a separate population and identified as a mixture of tumor cells and stromal cells to be used in reactivity assays described in Examples 4 and 5. After sorting, cells from the positive and negative (also referred to as unselected) sorted populations and the unsorted population were analyzed to verify purity and assess recovery rates. In an exemplary process, the PD-1+CD39+ purity after selection was about 80%.

TABLE E1

| (FX500 sort) | Phenotype |
|---|---|
| Selected cells | CD45ₚₒₛ, CD8ₚₒₛ and/or CD4ₚₒₛ, PD1ₚₒₛCD39ₚₒₛ |
| (positive) | |
| Unselected cells | CD45ₚₒₛ, CD8ₚₒₛ and/or CD4ₚₒₛ, PD1_{neg}CD39_{neg} |
| | CD45ₚₒₛ, CD8ₚₒₛ and/or CD4ₚₒₛ, PD1ₚₒₛCD39_{neg} |
| | CD45ₚₒₛ, CD8ₚₒₛ and/or CD4ₚₒₛ, PD1_{neg} CD39ₚₒₛ |

| Table E2 | |
|---|---|
| (MA900 sort) | Phenotype |
| Selected cells | 7-AAD_{neg} CD45ₚₒₛ, CD8ₚₒₛ and/or CD4ₚₒₛ, |
| (positive) | PD1ₚₒₛCD39ₚₒₛ |
| CD45negative cells | 7-AAD_{neg}CD45_{neg} |
| Unselected cells | CD45ₚₒₛ, CD8ₚₒₛ and/or CD4ₚₒₛ, PD1_{neg}CD39_{neg} |
| | CD45ₚₒₛ, CD8ₚₒₛ and/or CD4ₚₒₛ, PD1ₚₒₛCD39_{neg} |
| | CD45ₚₒₛ, CD8ₚₒₛ and/or CD4ₚₒₛ, PD1_{neg} CD39ₚₒₛ |

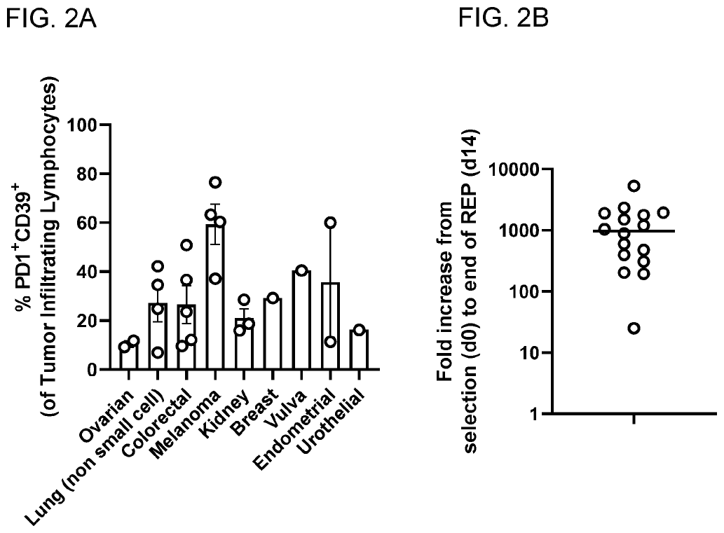

The percentage of PD1+CD39+ cells within the CD45+, CD4+ and/or CD8+ population TIL cell population is shown with respect to tumor type in FIG. 2A. PD1+ CD39+ TILs were detected at various frequencies (7-76% range) in tumors from all indications (e.g., types of tumors tested.

### Example 2: Post-Sort Expansion of Activated Tumor Derived T Cells

T cells sourced from primary tumors were processed as described in Example 1, and then selectively sorted as described in Example 2. Cells then underwent an in vitro expansion using a Rapid Expansion Protocol (REP) described below.

Freshly sorted PD1+CD39+ cells or their unselected counterparts were seeded into a gas permeable 24 well culture plate at 250,000-1,000,000 cells/cm2 in serum free OpTmizer medium supplemented with gentamicin at 10 pg/mL, and 2.0 mM of a L-alanyl-L-glutamine dipeptide form of glutamine (GlutaMAX Supplement; Thermofisher). Alternatively, this culture process can be performed in presence of human AB serum Cells were stimulated in the presence of allogenic feeder cells at a 200:1 Feeder (irradiated PBMC or iPBMC): TIL ratio, 30 ng/ml anti-CD3 antibody (OKT3 clone) and 3000 IU/ml of recombinant human IL-2 (Proleukin) in a G-Rex flask. Cells were incubated for a total of 14 days with media exchanges and splits performed to maintain optimal cell density and growth. Cell counts were performed using NC-200 Automated Cell Counter (Chemometec) on each culture day. Cells were split on day 7. After completion of the expansion phase, cells were washed in PBS then cryopreserved in the presence of a cryoprotectant. Cryopreservation was carried out using CoolCell devices (Corning) or the VIA Freeze (GE Healthcare).

As shown in FIG. 2B as fold expansion, each tumor infiltrating lymphocyte (TIL) T cell population tested underwent measurable expansion. PD1+CD39+ TILs were sorted from 16 of those samples (Ovarian, NSCLC, CRC, melanoma, breast, endometrial, urothelial, and kidney indications) and successfully expanded in vitro in a 14− day REP with an average expansion of 1000-fold observed across the different sample processed.

### Example 3: Determination of Phenotype and Clonalities of Expanded PD1+CD39+ TILs

The composition and phenotype of PD1+CD39+ selected TIL product from lung, colorectal, kidney, endometrial and melanoma tumors were further analyzed by flow cytometry. Following expansion using the REP described in Example 2, positive selected cells were collected and stained for various markers using the Aurora TIL-ACT Antibody Panel. The Aurora TIL-ACT Antibody Panel includes Live/Dead Blue viability marker (Thermo Fisher), anti-CD3 BV510 (BioLegend), anti-CD4 BUV395 (BD), anti-CD8 BUV805 (BD), anti-CD25 PE-Cy7 (BD), anti-FOXP3 AF647 (BioLegend), anti-TCR delta/gamma PerCP-Vio700 (Miltenyi), anti-CD56 BV570 (BioLegend), anti-CCR7 APC-Fire810 (BioLegend), anti-CD27 APC-H7 (BD), anti-CD28 BV650 (BioLegend), anti-CD45RA BUV563 (BD), anti-CD45RP eFluor450 (ThermoFisher), anti-CD95 BUV737 (BD), anti-CD127 APC-R700 (BD), anti-CD62L BV480 (BD), anti-TCF-1 BV421 (BD), anti-CXCR6 BB700 (BD), anti-CD57 FITC (BioLegend), anti-KLRG1 PE-Fire810 (BioLegend), anti-CD39 BUY661 (BD), anti-CD69 Spark NIR 685 (BioLegend), anti-CD103 (PE-Dazzle594 (BioLegend), anti-CD137 APC (BioLegend), anti-OX40 BV605 (BioLegend), anti-PD-1 BV785 (BioLegend), anti-TIGIT BV711 (BioLegend), anti-TIM3 PE (BioLegend), anti-LAG-3 PE-Cy5 (ThermoFisher).

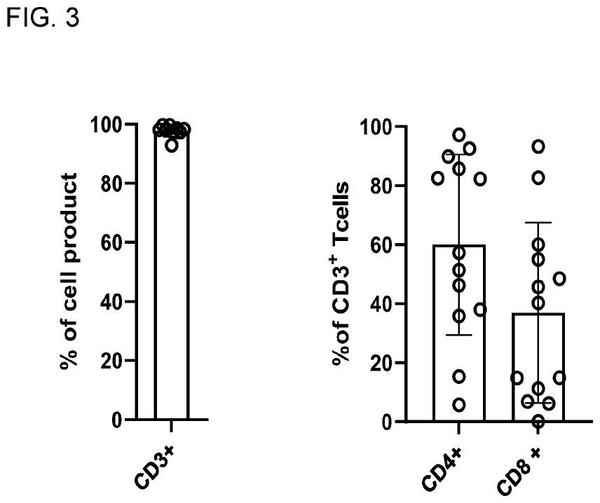

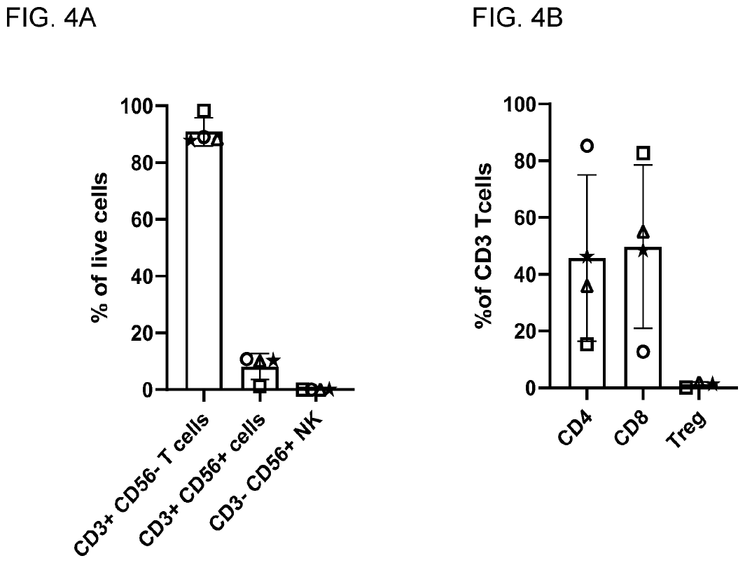

As shown in FIG. 3, flow cytometry analysis revealed that PD1+CD39+ selected TIL products are primarily composed of CD3+ T cells (>92%) and the percentage of CD4 and CD8 T cells are variable (CD3+ CD56− T cells represented the majority of PD1+CD39+ selected TIL product (>85%), while CD3+ CD56− T cells ranged between 1-11% and CD3− CD56+NK cells represented less than 0.1% (FIG. 4A). High variability in the percentage of CD4 and CD8 T cell subsets was observed across the PD1+CD39+ selected TIL products. Regulatory T cells (Treg), identified as CD4+ CD8− CD25+ Foxp3+ CD1271ow, were consistently detected at low frequencies (0.1-2%) (FIG. 4B).

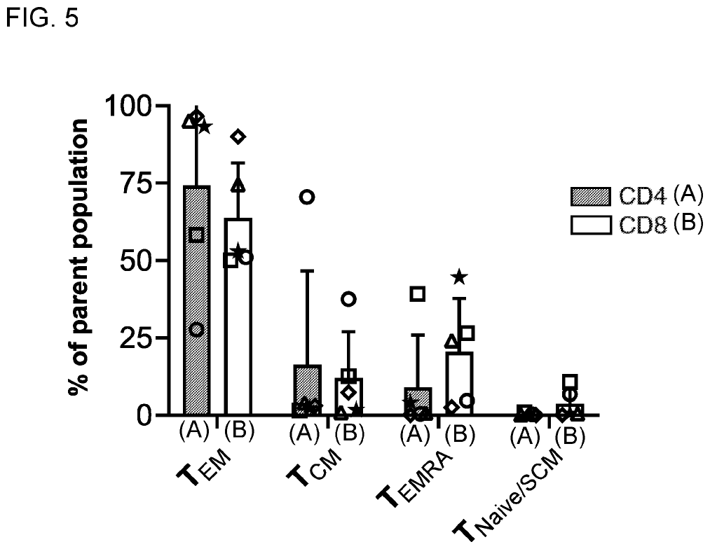

To assess the T cell phenotype, the expression of CD45RA and the chemokine receptor CCR7 were analyzed (FIG. 5). CD45RA− CCR7− cells are defined as effector memory T cells (Tem), CD45RA− CCR7+ cells are defined as central memory T cells (Tcm); CD45RA+CCR7− cells are defined as effector memory T cells; and CD45RA− CCR7− cells are defined as naïve/stem-cell memory T cells (Tnaive/SCM). This analysis revealed that CD4 and CD8 T cells in the PD1+CD39+ selected TIL product were primarily effector memory T cells (Tem). Central memory T cells (Tcm), a phenotype associated with increased in vivo persistence in animal models, also were detected at variable levels in different samples.

In addition to the phenotype characterization by flow cytometry, single cell RNA (scRNA) sequencing was performed on PD1+CD39+ selected and unselected (PD1−CD39+; PD1+CD39− and PD1−CD39−) TIL products (2 colorectal cancer, 2 melanomas, 1 non-small cell lung cancer and one ovarian cancer) to assess the TCR repertoire. To generate single cell sequencing data, the cells were processed according to manufacturer's instructions to generate libraries (10× Genomics) that were run on a sequencer to obtain information on the sequence and expression of genes expressed by each individual cells based on the sequences detected. The T cell clones were then defined as cells sharing unique association of TCR alpha and beta CDR3 sequences. Approximately, 1e5 live cells per group were washed, filtered through a Flowmi 40 μM cell strainer, and brought to a final concentration for sequencing (for 2000 cells) of 700-1300 cells/μl.

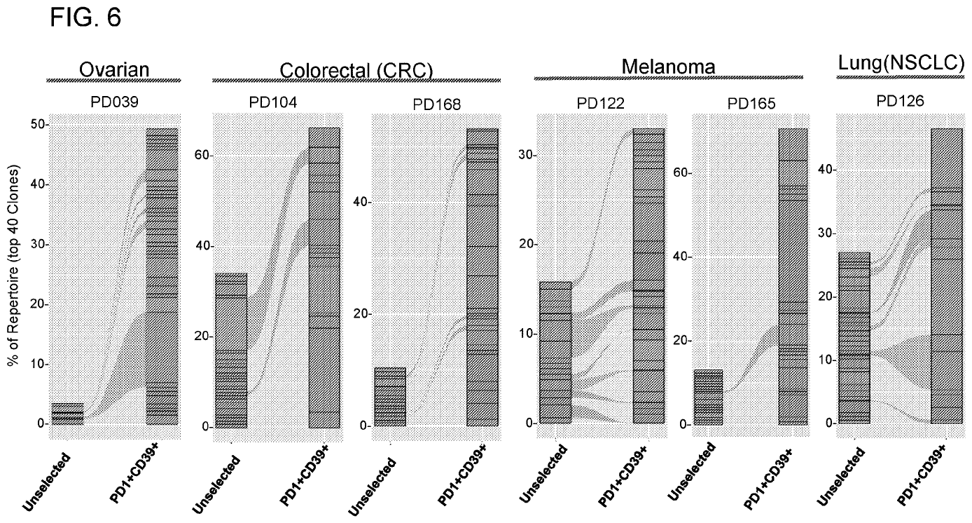

Minimal overlap between the top 40 most highly expressed clonotypes was observed between the TCR sequences in the unselected TIL and PD1+CD39+ selected TIL product, demonstrating that PD1+CD39+ direct selection enriches for a unique subset of tumor reactive TCRs (FIG. 6). PD1+CD39+ selected TIL are oligoclonal and show reduced clonotype diversity relative to unselected TIL, as shown by the higher percentage of the repertoire being made up of the top 40 most frequently expressed clonotypes.

### Example 4: Evaluation of PD1+CD39+ Cells from an Ovarian Tumor and their Functionality Against Autologous Tumor Material

The functionality of PD1+CD39+ selected TIL and unselected TIL from an ovarian tumor was assessed using two distinct functional assays in which the cells were either stimulated using polyclonal stimulation (anti-CD3 and anti-CD28 antibodies mediated stimulation) or antigen specific stimulation (autologous tumor cells). The results are described below.

### A. Polyclonal Stimulation

The polyclonal stimulation informs on the general ability of T cells to function as the anti-CD3 and anti-CD28 antibodies activate all live T cells, irrespective of their specificity. PD-1+CD39+ selected TILs and unselected TILs from the tumors were expanded and cryopreserved as described in Example 2. Cells were washed with OpTmizer cell culture media supplemented with 300 IU/mL recombinant IL-2, gentamicin at 10 μg/ml, Immune Cell Serum Replacement (ThermoFisher) at 5%, and a final concentration of 2.0 mM of a L-alanyl-L-glutamine dipeptide form of glutamine (GlutaMAX Supplement; Thermofisher). Cells were next seeded into 96-well culture plates to a final cell density of 1×10⁶ cells/mL with the addition of an anti-CD3/CD28 activator mix (end concentration: 25 μl/ml). Cells were stimulated overnight and supernatant was collected for cytokine analysis. Cytokine concentration was measured using the LegendPlex bead-based immunoassay according to the manufacturer's instructions.

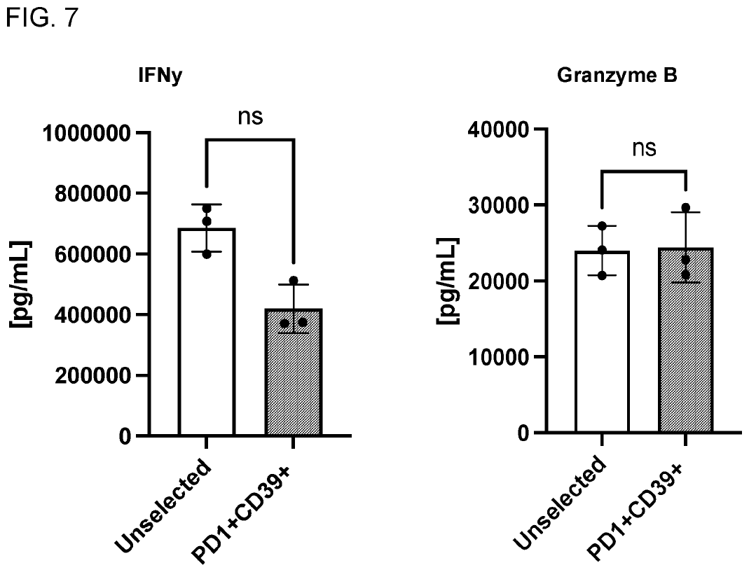

PD1+CD39+ selected TIL products generated from an ovarian tumor (PD039) produced IFNγ and Granzyme B after polyclonal stimulation at comparable levels to their unselected TIL counterparts (FIG. 7), demonstrating that even if selected based on the expression of terminal differentiation/exhaustion markers, PD1+CD39+ selected TIL products are functional after in vitro expansion.

### B. Antigen Specific Stimulation

Response to antigen specific stimulation was then assessed to determine the TIL's reactivity against the tumor. Autologous tumor cells (target cells) and TIL samples were thawed and diluted in OpTmizer cell culture media supplemented with 300 IU/mL recombinant IL-2, gentamicin at 10 μg/ml, Immune Cell Serum Replacement (ThermoFisher) at 5%, and a final concentration of 2.0 mM of a L-alanyl-L-glutamine dipeptide form of glutamine (GlutaMAX Supplement; Thermofisher). Cells were then co-cultured in a 96 well plate in the following combinations: negative control: TIL sample+anti-CD28 (2ug/ml)+anti-PD-1 (20ug/ml); positive control: TIL sample+anti-CD3/CD28 activator mix (25 ul/ml); Co-culture: TIL sample+target cells+anti-CD28 (2ug/ml)+anti-PD-1 (20ug/ml). Cells were incubated 37° C. and 5% C02 for 20-24 hours. After overnight co-culture, supernatant was collected and assessed by Legendplex for cytokine concentration, as described above. Cells were also washed and collected. Viability of the CD45 negative cells in the coculture (containing tumor cells) was assessed by flow cytometry and used to calculate tumor cell killing.

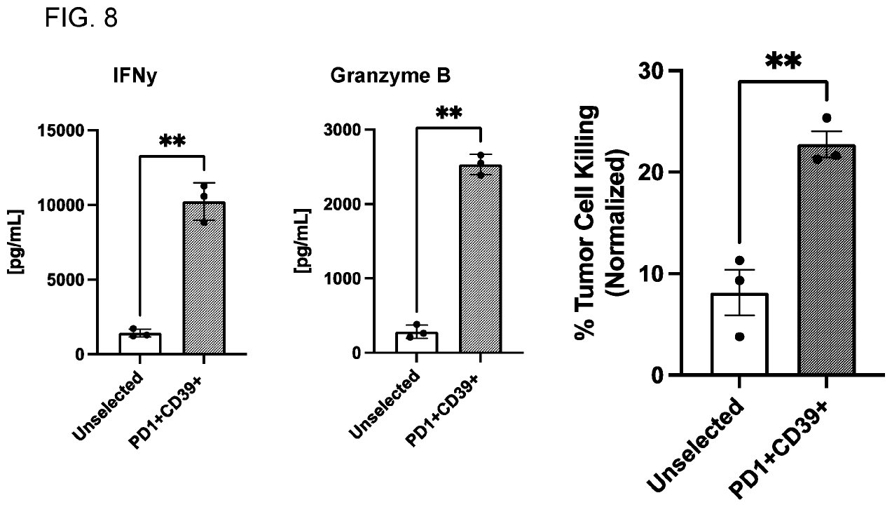

Upon coculture with autologous tumor cells, PD1+CD39+ selected TIL products showed a significant increase in tumor specific reactivity compared to the unselected TILs (PD1−CD39− double negative or PD1+CD39− or PD1−CD39+ single negative cells) as evidenced by the significant increase of IFNγ and Granzyme B production relative to the unselected TIL (FIG. 8). Notably, PD1+CD39+ selected TIL also demonstrated superior tumor cell killing compared to the unselected TIL, confirming that the PD1+CD39+ selection effectively enriches for tumor reactive TILs.

### Example 5: Evaluation of PD1+CD39+ Cells from a Colorectal Tumor and their Functionality Against Autologous Tumor Material

The functionality of PD1+CD39+ selected TIL and unselected TIL was assessed using two distinct functional assays in which the cells were either stimulated using polyclonal stimulation (anti-CD3 and anti-CD28 antibodies mediated stimulation) or antigen specific stimulation (autologous tumor cells) as described above.

### A. Polyclonal Stimulation

PD-1+CD39+ selected TILs and unselected TILs from the tumors were expanded and cryopreserved as described in Example 2. Cells were washed with OpTmizer cell culture media supplemented with 300 IU/mL recombinant IL-2, gentamicin at 10 μg/ml, Immune Cell Serum Replacement (ThermoFisher) at 5%, and a final concentration of 2.0 mM of a L-alanyl-L-glutamine dipeptide form of glutamine (GlutaMAX Supplement; Thermofisher). Cells were next seeded into 96-well culture plates to a final cell density of 1×10⁶ cells/mL with the addition of an anti-CD3/CD28 activator mix (end concentration: 25 μl/ml). Cells were stimulated overnight and supernatant was collected for cytokine analysis. Cytokine concentration was measured using the LegendPlex bead-based immunoassay according to the manufacturer's instructions.

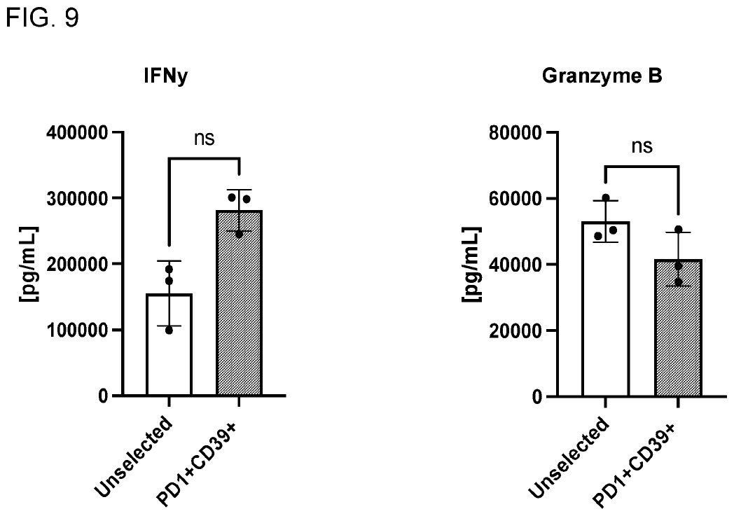

PD1+CD39+ selected TIL products generated from an ovarian tumor (PD039) produced IFNγ and Granzyme B after polyclonal stimulation at comparable levels to their unselected TIL counterparts (FIG. 9), demonstrating that even if selected based on the expression of terminal differentiation/exhaustion markers, PD1+CD39+ selected TIL products are functional after in vitro expansion.

### B. Antigen Specific Stimulation

Response to antigen specific stimulation was then assessed to determine the TIL's reactivity against the tumor. Autologous tumor cells (target cells) and TIL samples were thawed and diluted in OpTmizer cell culture media supplemented with 300 IU/mL recombinant IL-2, gentamicin at 10 μg/ml, Immune Cell Serum Replacement (ThermoFisher) at 5%, and a final concentration of 2.0 mM of a L-alanyl-L-glutamine dipeptide form of glutamine (GlutaMAX Supplement; Thermofisher). Cells were then co-cultured in a 96 well plate in the following combinations: negative control: TIL sample+anti-CD28 (2ug/ml)+anti-PD-1 (20ug/ml); positive control: TIL sample+anti-CD3/CD28 activator mix (25ul/ml); Co-culture: TIL sample+target cells+anti-CD28 (2ug/ml)+anti-PD-1 (20ug/ml). Cells were incubated 37° C. and 5% C02 for 20-24 hours. After overnight co-culture, supernatant was collected and assessed by Legenedplex for cytokine concentration, as described above. Cells were also washed and collected. Viability of the CD45 negative cells in the coculture (containing tumor cells) was assessed by flow cytometry and used to calculate tumor cell killing.

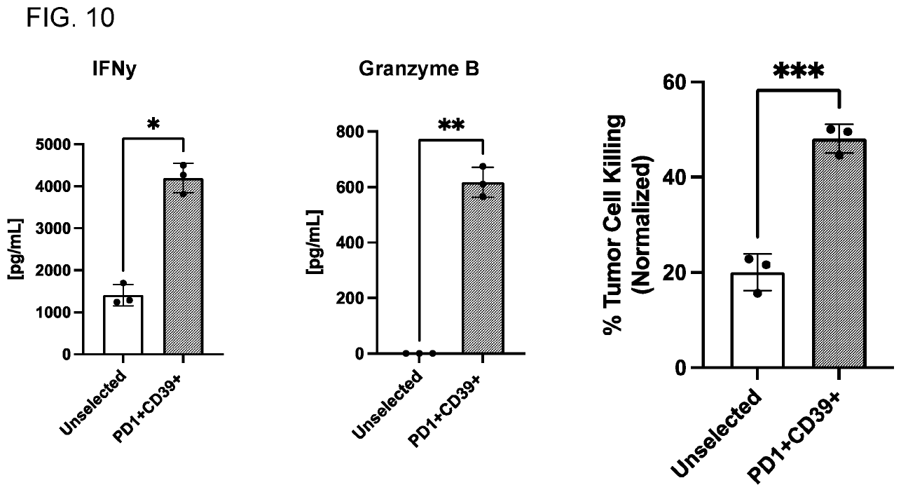

Upon coculture with autologous tumor cells, PD1+CD39+ selected TIL products showed a significant increase in tumor specific reactivity compared to the unselected TILs (PD1−CD39− double negative or PD1+CD39− or PD1−CD39+ single negative cells) as evidenced by the significant increase of IFNγ and Granzyme B production relative to the unselected TIL (FIG. 10). Notably, PD1+CD39+ selected TIL also demonstrated superior tumor cell killing compared to the unselected TIL, confirming that the PD1+CD39+ selection effectively enriches for tumor reactive TILs.

### VIII. The Conclusion

The foregoing descriptions of various embodiments of the invention have been presented for purposes of illustration and description. It is not intended to limit the invention to the precise forms disclosed. Many modifications, variations and refinements will be apparent to practitioners skilled in the art. For example, embodiments of the pharmaceutical T lymphocyte infiltrating (TIL) compositions described herein including those enriched in tumor reactive T cells can be adapted for treatment of a number of cancers including solid tumors such as GI, breast, bone and melanomas and various liquid/hematolgic tumors such as leukemia, lymphoma, multiple myeloma and related diseases. Also, those skilled in the art will recognize, or be able to ascertain using no more than routine experimentation, numerous equivalents to the specific devices and methods described herein. Such equivalents are considered to be within the scope of the present invention and are covered by the appended claims below.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Further still, embodiments of the invention also contemplate the exclusion or negative recitation of an element, feature, chemical, therapeutic agent, characteristic, value or step wherever said element, feature, chemical, therapeutic agent, characteristic, value, step or the like is positively recited. Hence, the scope of the present invention is not limited to the specifics of the described embodiments but is instead limited solely by the appended claims.

1. A pharmaceutical T lymphocyte infiltrating (TIL) composition enriched in tumor reactive T cells, the composition comprising a population of tumor infiltrating T cells comprising CD4+ and CD8+ T cells from a tumor, wherein at least 90% of cells in the composition are CD3+ T cells and less than about 5% of the population are T regulatory cells.

2. The pharmaceutical T lymphocyte infiltrating (TIL) composition of claim 1, wherein the TIL population is an oligoclonal population of tumor infiltrating T cells, and wherein up to 40 clones make up at least 40% of the TCR frequency in the population.

3-6. (canceled)

7. The pharmaceutical composition of claim 1, wherein T cells of the population express PD-1 and/or CD39.

8. The pharmaceutical composition of claim 7, wherein the percentage of cells that express surface marker PD-1 and/or CD39 in the composition, or of viable cells thereof, is about at least 30%, at least 40%, at least 50%, at least 60%, at least 70%, at least 80%, at least 90%, or at least 100%, of the cells in the composition.

9-14. (canceled)

15. The pharmaceutical composition of claim 1, wherein greater than 60% of the cells of the population are T effector memory cells.

16-19. (canceled)

20. The pharmaceutical composition of claim 15, wherein the effector memory phenotype is characterized by surface marker expression of one or more of CD45RA⁻, CD45RO+, CD62L⁻, CCR7−, CD28−, and CD27−.

21-23. (canceled)

24. The pharmaceutical composition of claim 2, wherein up to 40 TCR clonotypes make up at least 50% of the TCR frequency in the population, optionally wherein the top 40 clonotypes make up at least 50% of the TCR frequency in the population.

25. (canceled)

26. The pharmaceutical composition of claim 1, wherein the TCR clonotypes exhibit reactivity for at least one CD8 antigen and at least one CD4 antigen.

27. The pharmaceutical composition of claim 1, wherein at least 20% of the CD8+ T cells and/or at least 20% of the CD4+ T cells in the composition exhibit neoantigen reactivity.

28. The pharmaceutical composition of claim 1, wherein, the TIL composition is characterized by at least one of the following criteria in an in vitro autologous tumor assay:
i) IFN-γ production that is greater than 2000 pg/mL;
ii) granzyme B production in the supernatant that is greater than 200 pg/mL; and
iii) greater than 10% tumor cell killing.

29-36. (canceled)

37. The pharmaceutical composition of claim 1, wherein the composition is characterized by a greater number of CD4+ T cells than CD8+ T cells.

38-40. (canceled)

41. The pharmaceutical composition of claim 1, wherein the number of cells in the composition is a therapeutically effective amount of TILs.

42-53. (canceled)

54. The pharmaceutical composition of claim 1, wherein the composition is prepared by selecting cells surface positive for PD-1 and CD39 from cells obtained from a tumor of a donor subject and expanding the cells ex vivo.

55. The pharmaceutical composition of claim 1, wherein the composition is prepared by a method comprising:
a. providing dissociated tumor cells from a tumor obtained from a donor subject, wherein the dissociated tumor cells are a first population of cells that comprise CD4+ and CD8+ T cells;
b. selecting, from the first population of cells, cells that are surface positive for CD45, PD1 and CD39 and a T cell marker to produce a population of selected T lymphocyte infiltrating cells (TILs), optionally wherein the T cell marker is CD4 or CD8; and
c. expanding the population of selected TILs by culture with one or more T-cell stimulating agent of lymphocytes under conditions to produce a population of expanded T cells.

56. (canceled)

57. The method of claim 55, wherein the one or more T-cell stimulating agent is selected from one or more of allogenic feeder cells, anti-CD3 antibody, and recombinant IL-2.

58. The method of claim 55, wherein the dissociated tumor cells are a single cell suspension processed by homogenization and enzymatic digestion of one or more tumor fragments from the resected tumor.

59-63. (canceled)

64. A method of treating a subject having a cancer, the method comprising administering to a subject having a tumor a therapeutically effective dose of the composition of claim 1.

65-69. (canceled)

70. The method of claim 64, wherein the tumor is a colorectal cancer (CRC) tumor, a melanoma tumor, a non-small cell lung cancer (NSCLC) tumor, or an ovarian cancer tumor
